(19) United States
(12) Patent Application Publication
(10) Pub. No.: US 2021/0213078 A1
(43) Pub. Date: Jul. 15, 2021

(54) MICROBIAL COMPOSITIONS AND METHODS OF USE

(71) Applicant: Pendulum Therapeutics, Inc., San Francisco, CA (US)

(72) Inventors: John S. EID, San Francisco, CA (US); Colleen CUTCLIFFE, Menlo Park, CA (US); Orville KOLTERMAN, San Francisco, CA (US); Fanny PERRAUDEAU, San Francisco, CA (US); James BULLARD, San Francisco, CA (US); Paul MCMURDIE, San Francisco, CA (US); Andrew CHENG, San Francisco, CA (US); Marcus SCHICKLBERGER, San Francisco, CA (US); Nicholas JUSTICE, San Francisco, CA (US)

(21) Appl. No.: 17/210,078

(22) Filed: Mar. 23, 2021

Related U.S. Application Data
(63) Continuation of application No. PCT/US2019/052694, filed on Sep. 24, 2019.
(60) Provisional application No. 62/735,747, filed on Sep. 24, 2018.
(60) Provisional application No. 62/801,983, filed on Feb. 6, 2019.

(51) Int. Cl. A61K 35/747; A61K 9/00; A61K 35/742; A61K 35/741; A61K 35/745; A61K 35/744; A61K 31/155; A61P 3/10

(52) U.S. Cl. CPC A61K 35/747; A61K 9/0053; A61K 35/742; A61P 3/10; A61K 35/745; A61K 35/744; A61K 31/155; A61K 35/741

(57) ABSTRACT

Administering a composition comprising at least one mucin-regulating and at least one butyrate-producing microbe can provide a therapeutic effect for subjects having prediabetes or type 2 diabetes. Therapeutic effects can include a reduction in hemoglobin A1C levels, a reduction in glucose area under the curve after a meal tolerance test, or a reduction in the fasting glucose level.

### CROSS REFERENCE

This application claims the benefit of U.S. Provisional Patent Application No. 62/735,747, filed Sep. 24, 2018, and U.S. Provisional patent Application No. 62/801,983, filed Feb. 6, 2019, each of which is incorporated herein by reference in its entirety.

### BACKGROUND

Type 2 diabetes is a chronic disease that occurs when the body cannot effectively use the insulin it produces, and thus cannot regulate its blood sugar. Hyperglycemia, or high blood sugar, is a common effect of uncontrolled type 2 diabetes, and over time can lead to serious damage to many of the body's systems, especially the nerves and blood vessels. Many patients with diabetes fail to achieve glycemic control using currently available therapies.

### BIOLOGICAL DEPOSITS

This application contains a reference to a deposit of biological material. The following biological materials have been deposited with the American Type Culture Collection (ATCC), in Manassas, Va., and bear the following designations, accession numbers and dates of deposit: Clostridium beijerinckii; WB-STR-0005 (PTA-123634, deposited Dec. 14, 2016); Clostridium butyricum; WB-STR-0006 (PTA-123635, deposited Dec. 14, 2016).

### SUMMARY

Disclosed herein, in some aspects, is a method of treating a subject with an elevated hemoglobin A1C (hA1C) level, comprising administering to the subject a composition comprising at least one isolated and purified butyrate-producing microbe and at least one isolated and purified mucin-regulating microbe, thereby reducing the hA1C level in the subject by at least 0.2% of total hemoglobin.

In some embodiments, administering the composition reduces a glucose area under the curve (AUC) for the subject after a meal tolerance test by at least 10% relative to a control. In some embodiments, the control is a control AUC measured for the subject before the administering. In some embodiments, the control is a control AUC from a second subject that is not administered the composition. In some embodiments, the subject has or is suspected of having type 2 diabetes or prediabetes. In some embodiments, the at least one isolated and purified butyrate-producing microbe comprises one or more rRNA sequences with at least about 85% sequence identity to an rRNA sequence from any one or more of Clostridium beijerinckii, Eubacterium halli, and Clostridium butyricum. In some embodiments, the at least one isolated and purified butyrate-producing microbe comprises one or more rRNA sequences with at least about 90% sequence identity to an rRNA sequence from any one or more of Clostridium beijerinckii, Eubacterium hallii, and Clostridium butyricum. In some embodiments, the at least one isolated and purified butyrate-producing microbe comprises one or more rRNA sequences with at least about 97% sequence identity to an rRNA sequence from any one or more of Clostridium beijerinckii, Eubacterium hallii, and Clostridium butyricum. In some embodiments, the at least one isolated and purified butyrate-producing microbe comprises one or more microbes selected from the group consisting of Clostridium beijerinckii, Eubacterium hallii, and Clostridium butyricum. In some embodiments, the at least one isolated and purified mucin-regulating microbe comprises an rRNA sequence comprising at least about 85% sequence identity to an rRNA sequence of Akkermansia muciniphila. In some embodiments, the at least one isolated and purified mucin-regulating microbe comprises an rRNA sequence comprising at least about 90% sequence identity to an rRNA sequence of Akkermansia muciniphila. In some embodiments, the at least one isolated and purified mucin-regulating microbe comprises an rRNA sequence comprising at least about 97% sequence identity to an rRNA sequence of Akkermansia muciniphila. In some embodiments, the at least one isolated and purified mucin-regulating microbe comprises an rRNA sequence comprising at least about 97% sequence identity to any one of SEQ ID NOS: 1-6. In some embodiments, the at least one isolated and purified mucin-regulating microbe comprises an rRNA sequence comprising at least about 99% sequence identity to any one of SEQ ID NOS: 1-6. In some embodiments, the at least one isolated and purified mucin-regulating microbe comprises an rRNA sequence that is any one of SEQ ID NOS: 1-6. In some embodiments, the at least one isolated and purified mucin-regulating microbe comprises Akkermansia muciniphila. In some embodiments, the subject has the type 2 diabetes. In some embodiments, the subject has the prediabetes. In some embodiments, the type 2 diabetes is early stage. In some embodiments, the type 2 diabetes is mid stage. In some embodiments, the type 2 diabetes is late stage. In some embodiments, the composition further comprises metformin. In some embodiments, the composition is co-administered with a therapeutic agent. In some embodiments, the therapeutic agent is metformin. In some embodiments, the therapeutic agent is sulfonylurea. In some embodiments, the therapeutic agent is insulin. In some embodiments, the composition comprises a therapeutic agent. In some embodiments, the therapeutic agent is metformin. In some embodiments, the therapeutic agent is sulfonylurea. In some embodiments, the therapeutic agent is insulin. In some embodiments, the hA1C level is reduced in the subject by at least 0.2% of total hemoglobin. In some embodiments, the hA1C level is reduced in the subject by at least 0.3% of total hemoglobin. In some embodiments, the hA1C level is reduced in the subject by at least 0.4% of total hemoglobin. In some embodiments, the hA1C level is reduced in the subject by at least 0.5% of total hemoglobin. In some embodiments, the hA1C level is reduced in the subject by at least 0.6% of total hemoglobin. In some embodiments, the hA1C level is reduced in the subject by at least 0.2% of total hemoglobin relative to a second subject that is not administered the composition. In some embodiments, the hA1C level is reduced in the subject by at least 0.3% of total hemoglobin relative to a second subject that is not administered the composition. In some embodiments, the hA1C level is reduced in the subject by at least 0.4% of total hemoglobin relative to a second subject that is not administered the composition. In some embodiments, the hA1C level is reduced in the subject by at least 0.5% of total hemoglobin relative to a second subject that is not administered the composition. In some embodiments, the hA1C level is reduced in the subject by at least 0.6% of total hemoglobin relative to a second subject that is not administered the composition. In some embodiments, the glucose AUC is reduced by at least 10%. In some embodiments, the glucose AUC is reduced by at least 15%. In some embodiments, the glucose AUC is reduced by at least 20%. In some embodiments, the glucose AUC is reduced by at least 30%. In some embodiments, fasting glucose is reduced in the subject by at least 5%. In some embodiments, fasting glucose is reduced in the subject by at least 10%. In some embodiments, fasting glucose is reduced in the subject by at least 20%. In some embodiments, fasting glucose is reduced in the subject by at least 25%. In some embodiments, the subject is a human. In some embodiments, the subject has a comorbidity. In some embodiments, the composition comprises one or more microbes with an rRNA sequence comprising at least about 85% sequence identity to an rRNA sequence of Clostridium beijerinckii. In some embodiments, the composition comprises one or more microbes with an rRNA sequence comprising at least about 85% sequence identity to an rRNA sequence of Clostridium butyricum. In some embodiments, the composition comprises one or more microbes with an rRNA sequence comprising at least about 85% sequence identity to an rRNA sequence of Bifidobacterium infantis. In some embodiments, the composition comprises one or more microbes with an rRNA sequence comprising at least about 85% sequence identity to an rRNA sequence of Eubacterium halli. In some embodiments, the composition comprises one or more microbes with an rRNA sequence comprising at least about 85% sequence identity to an rRNA sequence of Akkermansia muciniphila. In some embodiments, the composition comprises one or more microbes with an rRNA sequence comprising at least about 90% sequence identity to an rRNA sequence of Clostridium beijerinckii. In some embodiments, the composition comprises one or more microbes with an rRNA sequence comprising at least about 90% sequence identity to an rRNA sequence of Clostridium butyricum. In some embodiments, the composition comprises one or more microbes with an rRNA sequence comprising at least about 90% sequence identity to an rRNA sequence of Bifidobacterium infantis. In some embodiments, the composition comprises one or more microbes with an rRNA sequence comprising at least about 90% sequence identity to an rRNA sequence of Eubacterium hallii. In some embodiments, the composition comprises one or more microbes with an rRNA sequence comprising at least about 90% sequence identity to an rRNA sequence of Akkermansia muciniphila. In some embodiments, the composition comprises one or more microbes with an rRNA sequence comprising at least about 97% sequence identity to an rRNA sequence of Clostridium beijerinckii. In some embodiments, the composition comprises one or more microbes with an rRNA sequence comprising at least about 97% sequence identity to an rRNA sequence of Clostridium butyricum. In some embodiments, the composition comprises one or more microbes with an rRNA sequence comprising at least about 97% sequence identity to an rRNA sequence of Bifidobacterium infantis. In some embodiments, the composition comprises one or more microbes with an rRNA sequence comprising at least about 97% sequence identity to an rRNA sequence of Eubacterium hallii. In some embodiments, the composition comprises one or more microbes with an rRNA sequence comprising at least about 97% sequence identity to an rRNA sequence of Akkermansia muciniphila. In some embodiments, the composition comprises Clostridium beijerinckii. In some embodiments, the composition comprises Clostridium butyricum. In some embodiments, the composition comprises Bifidobacterium infantis. In some embodiments, the composition comprises Akkermansia muciniphila. In some embodiments, the composition comprises Eubacterium hallii. In some embodiments, the composition comprises Clostridium beijerinckii, Clostridium butyricum, and Bifidobacterium infantis. In some embodiments, the composition comprises Clostridium beijerinckii, Clostridium butyricum, Bifidobacterium infantis, Akkermansia muciniphila, and Eubacterium hallii. In some embodiments, the composition comprises Clostridium butyricum, Bifidobacterium infantis, Akkermansia muciniphila, and Eubacterium hallii. In some embodiments, the composition comprises Clostridium beijerinckii, Bifidobacterium infantis, Akkermansia muciniphila, and Eubacterium hallii. In some embodiments, the composition comprises Clostridium beijerinckii, Akkermansia muciniphila, and Eubacterium hallii. In some embodiments, the composition comprises Clostridium beijerinckii and Bifidobacterium infantis. In some embodiments, the composition comprises Clostridium beijerinckii, Clostridium butyricum, Bifidobacterium infantis, Akkermansia muciniphila, and Eubacterium hallii. In some embodiments, the composition comprises Clostridium beijerinckii, Clostridium butyricum, Bifidobacterium infantis, and Akkermansia muciniphila. In some embodiments, the composition comprises Clostridium butyricum, Bifidobacterium infantis, and Akkermansia muciniphila. In some embodiments, the composition comprises Eubacterium hallii and Akkermansia muciniphila. In some embodiments, the composition comprises Bifidobacterium infantis, Eubacterium hallii, and Akkermansia muciniphila. In some embodiments, the composition comprises at least 2 microbes. In some embodiments, the composition comprises at least 3 microbes. In some embodiments, the composition comprises at least 4 microbes. In some embodiments, the composition comprises at least 5 microbes. In some embodiments, the composition comprises at least 2 microbes selected from the group consisting of Clostridium beijerinckii, Clostridium butyricum, Biidobacterium infantis, Akkermansia muciniphila, and Eubacterium hallii. In some embodiments, the composition comprises at least 3 microbes selected from the group consisting of Clostridium beijerinckii, Clostridium butyricum, Bifidobacterium infantis, Akkermansia muciniphila, and Eubacterium hallii. In some embodiments, the composition comprises at least 4 microbes selected from the group consisting of Clostridium beijerinckii, Clostridium butyricum, Bifidobacterium infantis, Akkermansia muciniphila, and Eubacterium hallii. In some embodiments, the composition is in a unit dosage form. In some embodiments, the composition is a food or beverage. In some embodiments, the composition is a dietary supplement. In some embodiments, the dietary supplement is in a form of a food bar. In some embodiments, the dietary supplement is in a form of a powder. In some embodiments, the dietary supplement is in a form of a liquid. In some embodiments, the composition is a pharmaceutical composition. In some embodiments, the composition is in a form of a pill or capsule. In some embodiments, the pill or capsule comprises an enteric coating designed to release the contents of the pill or capsule in an ileum of the subject, a colon of the subject, or a combination thereof. In some embodiments, each pill or capsule comprises at least 1×10⁶ CFU of total microbes. In some embodiments, each pill or capsule comprises at least 1×10⁶ CFU of the at least one isolated and purified mucin-regulating microbe. In some embodiments, each pill or capsule comprises at least 1×10⁶ CFU of the at least one isolated and purified butyrate-producing microbe. In some embodiments, each pill or capsule comprises at least 1×10⁶ CFU of Akkermansia muciniphila, a microbe comprising an rRNA sequence with at least about 97% sequence identity to an rRNA from Akkermansia muciniphila, or a microbe comprising an rRNA sequence with at least about 97% sequence identity to any one of SEQ ID NOS: 1-6. In some embodiments, each pill or capsule comprises at least 1×10⁶ CFU of Eubacterium hallii or a microbe comprising an rRNA sequence with at least about 97% sequence identity to an rRNA from Eubacterium hallii. In some embodiments, each pill or capsule comprises at least 1×10⁶ CFU of Bifidobacterium infantis or a microbe comprising an rRNA sequence with at least about 97% sequence identity to an rRNA from Bifidobacterium infantis. In some embodiments, each pill or capsule comprises at least 1×10⁶ CFU of Clostridium beijerinckii or a microbe comprising an rRNA sequence with at least about 97% sequence identity to an rRNA from Clostridium beijerinckii. In some embodiments, each pill or capsule comprises at least 1×10⁶ CFU of Clostridium butyricum or a microbe comprising an rRNA sequence with at least about 97% sequence identity to an rRNA from Clostridium butyricum. In some embodiments, each pill or capsule comprises between about 1×10⁶ CFU and 1×10¹² CFU of total microbes. In some embodiments, each pill or capsule comprises between about 1×10⁶ CFU and 1×10¹² CFU of the at least one isolated and purified mucin-regulating microbe. In some embodiments, each pill or capsule comprises between about 1×10⁶ CFU and 1×10¹² CFU of the at least one isolated and purified butyrate-producing microbe. In some embodiments, each pill or capsule comprises between about 1×10⁶ CFU and 1×10¹² CFU of Akkermansia muciniphila, a microbe comprising an rRNA sequence with at least about 97% sequence identity to an rRNA from Akkermansia muciniphila, or a microbe comprising an rRNA sequence with at least about 97% sequence identity to any one of SEQ ID NOS: 1-6. In some embodiments, each pill or capsule comprises between about 1×10⁶ CFU and 1×10¹² CFU of Eubacterium hallii or a microbe comprising an rRNA sequence with at least about 97% sequence identity to an rRNA from Eubacterium hallii. In some embodiments, each pill or capsule comprises between about 1×10⁶ CFU and 1×10¹² CFU of Bifidobacterium infantis or a microbe comprising an rRNA sequence with at least about 97% sequence identity to an rRNA from Bifidobacterium infantis. In some embodiments, each pill or capsule comprises between about 1×10⁶ CFU and 1×10¹² CFU of Clostridium beijerinckii or a microbe comprising an rRNA sequence with at least about 97% sequence identity to an rRNA from Clostridium beijerinckii. In some embodiments, each pill or capsule comprises between about 1×10⁶ CFU and 1×10¹² CFU of Clostridium butyricum or a microbe comprising an rRNA sequence with at least about 97% sequence identity to an rRNA from Clostridium butyricum. In some embodiments, one dose of the composition comprises at least one of the one pills or capsules. In some embodiments, one dose of the composition comprises at least two of the pills or capsules. In some embodiments, one dose of the composition comprises one to six of the pills or capsules. In some embodiments, the composition is administered to the subject at least weekly. In some embodiments, the composition is administered to the subject at least daily. In some embodiments, the composition is administered to the subject at least twice a day. In some embodiments, each dose of the composition comprises at least 1×10⁶ CFU of total microbes. In some embodiments, each dose of the composition comprises at least 1×10⁶ CFU of the at least one isolated and purified mucin-regulating microbe. In some embodiments, each dose of the composition comprises at least 1×10⁶ CFU of the at least one isolated and purified butyrate-producing microbe. In some embodiments, each dose of the composition comprises at least 1×10⁶ CFU of Akkermansia muciniphila, a microbe comprising an rRNA sequence with at least about 97% sequence identity to an rRNA from Akkermansia muciniphila, or a microbe comprising an rRNA sequence with at least about 97% sequence identity to any one of SEQ ID NOS: 1-6. In some embodiments, each dose of the composition comprises at least 1×10⁶ CFU of Eubacterium hallii or a microbe comprising an rRNA sequence with at least about 97% sequence identity to an rRNA from Eubacterium hallii. In some embodiments, each dose of the composition comprises at least 1×10⁶ CFU of Bifidobacterium infantis or a microbe comprising an rRNA sequence with at least about 97% sequence identity to an rRNA from Bifidobacterium infantis. In some embodiments, each dose of the composition comprises at least 1×10⁶ CFU of Clostridium beijerinckii or a microbe comprising an rRNA sequence with at least about 97% sequence identity to an rRNA from Clostridium beijerinckii. In some embodiments, each dose of the composition comprises at least 1×10⁶ CFU of Clostridium butyricum or a microbe comprising an rRNA sequence with at least about 97% sequence identity to an rRNA from Clostridium butyricum. In some embodiments, each dose of the composition comprises between about 1×10⁶ CFU and 1×10¹² CFU of total microbes. In some embodiments, each dose of the composition comprises between about 1×10⁶ CFU and 1×10¹² CFU of the at least one isolated and purified mucin-regulating microbe. In some embodiments, each dose of the composition comprises between about 1×10⁶ CFU and 1×10¹² CFU of the at least one isolated and purified butyrate-producing microbe. In some embodiments, each dose of the composition comprises between about 1×10⁶ CFU and 1×10¹² CFU of Akkermansia muciniphila, a microbe comprising an rRNA sequence with at least about 97% sequence identity to an rRNA from Akkermansia muciniphila, or a microbe comprising an rRNA sequence with at least about 97% sequence identity to any one of SEQ ID NOS: 1-6. In some embodiments, each dose of the composition comprises between about 1×10⁶ CFU and 1×10¹² CFU of Eubacterium hallii or a microbe comprising an rRNA sequence with at least about 97% sequence identity to an rRNA from Eubacterium hallii. In some embodiments, each dose of the composition comprises between about 1×10⁶ CFU and 1×10¹² CFU of Bifidobacterium infantis or a microbe comprising an rRNA sequence with at least about 97% sequence identity to an rRNA from Bifidobacterium infantis. In some embodiments, each dose of the composition comprises between about 1×10⁶ CFU and 1×10¹² CFU of Clostridium beijerinckii or a microbe comprising an rRNA sequence with at least about 97% sequence identity to an rRNA from Clostridium beijerinckii. In some embodiments, each dose of the composition comprises between about 1×10⁶ CFU and 1×10¹² CFU of Clostridium butyricum or a microbe comprising an rRNA sequence with at least about 97% sequence identity to an rRNA from Clostridium butyricum. In some embodiments, prior to the administering, the subject exhibits a fasting blood glucose level of at least about 125 mg/dL. In some embodiments, prior to the administering, the subject exhibits a blood glucose level after a glucose tolerance test of at least about 200 mg/dL. In some embodiments, prior to the administering, the subject exhibits a postprandial glucose level of at least about 200 mg/dL between about 1.5 and 2.5 hours after a meal. In some embodiments, prior to the administering, the subject exhibits a hA1C level of at least 6.4% of total hemoglobin. In some embodiments, prior to the administering, the subject exhibits a fasting blood glucose level of at least about 100 mg/dL. In some embodiments, prior to the administering, the subject exhibits a blood glucose level after a glucose tolerance test of at least about 140 mg/dL. In some embodiments, prior to the administering, the subject exhibits a postprandial glucose level of at least about 140 mg/dL between about 1.5 and 2.5 hours after a meal. In some embodiments, prior to the administering, the subject exhibits a hA1C level of at least 5.7% of total hemoglobin. In some embodiments, prior to the administering, the subject exhibits a fasting blood glucose level of less than about 100 mg/dL. In some embodiments, prior to the administering, the subject exhibits a blood glucose level after a glucose tolerance test of less than about 140 mg/dL. In some embodiments, prior to the administering, the subject exhibits a postprandial glucose level of less than about 140 mg/dL between about 1.5 and 2.5 hours after a meal. In some embodiments, prior to the administering, the subject exhibits a hA1C level of less than 5.7% of total hemoglobin. In some embodiments, insulin sensitivity is increased in the subject. In some embodiments, blood glucose levels are stabilized in the subject. In some embodiments, metabolic syndrome is treated in the subject. In some embodiments, insulin resistance is treated in the subject.

Disclosed herein, in some aspects, is a method of treating prediabetes in a subject, comprising administering to the subject a composition comprising at least one isolated and purified butyrate-producing microbe and at least one isolated and purified mucin-regulating microbe, thereby treating the prediabetes in the subject.

In some embodiments, the composition reduces a hemoglobin A1C (hA1C) level in the subject by at least 0.1% of total hemoglobin. In some embodiments, administering the composition reduces a glucose area under the curve (AUC) for the subject after a meal tolerance test by at least 10% relative to a control. In some embodiments, the control is a control AUC measured for the subject before the administering. In some embodiments, the control is a control AUC from a second subject that is not administered the composition. In some embodiments, prior to the administering, the subject exhibits a fasting blood glucose level of between about 100 mg/dL and 125 mg/dL. In some embodiments, prior to the administering, the subject exhibits a blood glucose level after a glucose tolerance test of between about 140 mg/dL and 199 mg/dL. In some embodiments, prior to the administering, the subject exhibits a hA1C level of between about 5.7 and 6.4% of total hemoglobin. In some embodiments, prior to the administering, the subject exhibits a postprandial glucose level of between about 140 mg/dL and 199 mg/dL between about 1.5 and 2.5 hours after a meal. In some embodiments, the subject has been prediabetic for at least 1 month. In some embodiments, the at least one isolated and purified butyrate-producing microbe comprises one or more rRNA sequences with at least about 85% sequence identity to an rRNA sequence from any one or more of Clostridium beijerinckii, Eubacterium hallii, and Clostridium butyricum. In some embodiments, the at least one isolated and purified butyrate-producing microbe comprises one or more rRNA sequences with at least about 90% sequence identity to an rRNA sequence from any one or more of Clostridium beijerinckii, Eubacterium hallii, and Clostridium butyricum. In some embodiments, the at least one isolated and purified butyrate-producing microbe comprises one or more rRNA sequences with at least about 97% sequence identity to an rRNA sequence from any one or more of Clostridium beijerinckii, Eubacterium hallii, and Clostridium butyricum. In some embodiments, the at least one isolated and purified butyrate-producing microbe comprises one or more microbes selected from the group consisting of Clostridium beijerinckii, Eubacterium hallii, and Clostridium butyricum. In some embodiments, the at least one isolated and purified mucin-regulating microbe comprises an rRNA sequence comprising at least about 85% sequence identity to an rRNA sequence of Akkermansia muciniphila. In some embodiments, the at least one isolated and purified mucin-regulating microbe comprises an rRNA sequence comprising at least about 90% sequence identity to an rRNA sequence of Akkermansia muciniphila. In some embodiments, the at least one isolated and purified mucin-regulating microbe comprises an rRNA sequence comprising at least about 97% sequence identity to an rRNA sequence of Akkermansia muciniphila. In some embodiments, the at least one isolated and purified mucin-regulating microbe comprises an rRNA sequence comprising at least about 97% sequence identity to any one of SEQ ID NOS: 1-6. In some embodiments, the at least one isolated and purified mucin-regulating microbe comprises an rRNA sequence comprising at least about 99% sequence identity to any one of SEQ ID NOS: 1-6. In some embodiments, the at least one isolated and purified mucin-regulating microbe comprises an rRNA sequence that is any one of SEQ ID NOS: 1-6. In some embodiments, the at least one isolated and purified mucin-regulating microbe comprises Akkermansia muciniphila. In some embodiments, the composition further comprises metformin. In some embodiments, the composition is co-administered with a therapeutic agent. In some embodiments, the therapeutic agent is metformin. In some embodiments, the therapeutic agent is sulfonylurea. In some embodiments, the therapeutic agent is insulin. In some embodiments, the composition comprises a therapeutic agent. In some embodiments, the therapeutic agent is metformin. In some embodiments, the therapeutic agent is sulfonylurea. In some embodiments, the therapeutic agent is insulin. In some embodiments, the hA1C level is reduced in the subject by at least 0.2% of total hemoglobin. In some embodiments, the hA1C level is reduced in the subject by at least 0.3% of total hemoglobin. In some embodiments, the hA1C level is reduced in the subject by at least 0.4% of total hemoglobin. In some embodiments, the hA1C level is reduced in the subject by at least 0.5% of total hemoglobin. In some embodiments, the hA1C level is reduced in the subject by at least 0.6% of total hemoglobin. In some embodiments, the hA1C level is reduced in the subject by at least 0.2% of total hemoglobin relative to a second subject that is not administered the composition. In some embodiments, the hA1C level is reduced in the subject by at least 0.3% of total hemoglobin relative to a second subject that is not administered the composition. In some embodiments, the hA1C level is reduced in the subject by at least 0.4% of total hemoglobin relative to a second subject that is not administered the composition. In some embodiments, the hA1C level is reduced in the subject by at least 0.5% of total hemoglobin relative to a second subject that is not administered the composition. In some embodiments, the hA1C level is reduced in the subject by at least 0.6% of total hemoglobin relative to a second subject that is not administered the composition. In some embodiments, the glucose AUC is reduced by at least 10%. In some embodiments, the glucose AUC is reduced by at least 15%. In some embodiments, the glucose AUC is reduced by at least 20%. In some embodiments, the glucose AUC is reduced by at least 30%. In some embodiments, fasting glucose is reduced in the subject by at least 5%. In some embodiments, fasting glucose is reduced in the subject by at least 10%. In some embodiments, fasting glucose is reduced in the subject by at least 20%. In some embodiments, fasting glucose is reduced in the subject by at least 25%. In some embodiments, the subject is a human. In some embodiments, the subject has a comorbidity. In some embodiments, the composition comprises one or more microbes with an rRNA sequence comprising at least about 85% sequence identity to an rRNA sequence of Clostridium beijerinckii. In some embodiments, the composition comprises one or more microbes with an rRNA sequence comprising at least about 85% sequence identity to an rRNA sequence of Clostridium butyricum. In some embodiments, the composition comprises one or more microbes with an rRNA sequence comprising at least about 85% sequence identity to an rRNA sequence of Bifidobacterium infantis. In some embodiments, the composition comprises one or more microbes with an rRNA sequence comprising at least about 85% sequence identity to an rRNA sequence of Eubacterium hallii. In some embodiments, the composition comprises one or more microbes with an rRNA sequence comprising at least about 85% sequence identity to an rRNA sequence of Akkermansia muciniphila. In some embodiments, the composition comprises one or more microbes with an rRNA sequence comprising at least about 90% sequence identity to an rRNA sequence of Clostridium beijerinckii. In some embodiments, the composition comprises one or more microbes with an rRNA sequence comprising at least about 90% sequence identity to an rRNA sequence of Clostridium butyricum. In some embodiments, the composition comprises one or more microbes with an rRNA sequence comprising at least about 90% sequence identity to an rRNA sequence of Bifidobacterium infantis. In some embodiments, the composition comprises one or more microbes with an rRNA sequence comprising at least about 90% sequence identity to an rRNA sequence of Eubacterium hallii. In some embodiments, the composition comprises one or more microbes with an rRNA sequence comprising at least about 90% sequence identity to an rRNA sequence of Akkermansia muciniphila. In some embodiments, the composition comprises one or more microbes with an rRNA sequence comprising at least about 97% sequence identity to an rRNA sequence of Clostridium beijerinckii. In some embodiments, the composition comprises one or more microbes with an rRNA sequence comprising at least about 97% sequence identity to an rRNA sequence of Clostridium butyricum. In some embodiments, the composition comprises one or more microbes with an rRNA sequence comprising at least about 97% sequence identity to an rRNA sequence of Bifidobacterium infantis. In some embodiments, the composition comprises one or more microbes with an rRNA sequence comprising at least about 97% sequence identity to an rRNA sequence of Eubacterium hallii. In some embodiments, the composition comprises one or more microbes with an rRNA sequence comprising at least about 97% sequence identity to an rRNA sequence of Akkermansia muciniphila. In some embodiments, the composition comprises Clostridium beijerinckii. In some embodiments, the composition comprises Clostridium butyricum. In some embodiments, the composition comprises Bifidobacterium infantis. In some embodiments, the composition comprises Akkermansia muciniphila. In some embodiments, the composition comprises Eubacterium hallii. In some embodiments, the composition comprises Clostridium beijerinckii, Clostridium butyricum, and Bifidobacterium infantis. In some embodiments, the composition comprises Clostridium beijerinckii, Clostridium butyricum, Bifidobacterium infantis, Akkermansia muciniphila, and Eubacterium hallii. In some embodiments, the composition comprises Clostridium butyricum, Bifidobacterium infantis, Akkermansia muciniphila, and Eubacterium hallii. In some embodiments, the composition comprises Clostridium beijerinckii, Bifidobacterium infantis, Akkermansia muciniphila, and Eubacterium hallii. In some embodiments, the composition comprises Clostridium beijerinckii, Akkermansia muciniphila, and Eubacterium hallii. In some embodiments, the composition comprises Clostridium beijerinckii and Bifidobacterium infantis. In some embodiments, the composition comprises Clostridium beijerinckii, Clostridium butyricum, Bifidobacterium infantis, Akkermansia muciniphila, and Eubacterium hallii. In some embodiments, the composition comprises Clostridium beijerinckii, Clostridium butyricum, Bifidobacterium infantis, and Akkermansia muciniphila. In some embodiments, the composition comprises Clostridium butyricum, Bifidobacterium infantis, and Akkermansia muciniphila. In some embodiments, the composition comprises Eubacterium hallii and Akkermansia muciniphila. In some embodiments, the composition comprises Bifidobacterium infantis, Eubacterium hallii, and Akkermansia muciniphila. In some embodiments, the composition comprises at least 2 microbes. In some embodiments, the composition comprises at least 3 microbes. In some embodiments, the composition comprises at least 4 microbes. In some embodiments, the composition comprises at least 5 microbes. In some embodiments, the composition comprises at least 2 microbes selected from the group consisting of Clostridium beijerinckii, Clostridium butyricum, Biidobacterium infantis, Akkermansia muciniphila, and Eubacterium hallii. In some embodiments, the composition comprises at least 3 microbes selected from the group consisting of Clostridium beijerinckii, Clostridium butyricum, Bifidobacterium infantis, Akkermansia muciniphila, and Eubacterium hallii. In some embodiments, the composition comprises at least 4 microbes selected from the group consisting of Clostridium beijerinckii, Clostridium butyricum, Bifidobacterium infantis, Akkermansia muciniphila, and Eubacterium hallii. In some embodiments, the composition is in a unit dosage form. In some embodiments, the composition is a food or beverage. In some embodiments, the composition is a dietary supplement. In some embodiments, the dietary supplement is in a form of a food bar. In some embodiments, the dietary supplement is in a form of a powder. In some embodiments, the dietary supplement is in a form of a liquid. In some embodiments, the composition is a pharmaceutical composition. In some embodiments, the composition is in a form of a pill or capsule. In some embodiments, the pill or capsule comprises an enteric coating designed to release the contents of the pill or capsule in an ileum of the subject, a colon of the subject, or a combination thereof. In some embodiments, each pill or capsule comprises at least 1×10⁶ CFU of total microbes. In some embodiments, each pill or capsule comprises at least 1×10⁶ CFU of the at least one isolated and purified mucin-regulating microbe. In some embodiments, each pill or capsule comprises at least 1×10⁶ CFU of the at least one isolated and purified butyrate-producing microbe. In some embodiments, each pill or capsule comprises at least 1×10⁶ CFU of Akkermansia muciniphila, a microbe comprising an rRNA sequence with at least about 97% sequence identity to an rRNA from Akkermansia muciniphila, or a microbe comprising an rRNA sequence with at least about 97% sequence identity to any one of SEQ ID NOS: 1-6. In some embodiments, each pill or capsule comprises at least 1×10⁶ CFU of Eubacterium hallii or a microbe comprising an rRNA sequence with at least about 97% sequence identity to an rRNA from Eubacterium hallii. In some embodiments, each pill or capsule comprises at least 1×10⁶ CFU of Bifidobacterium infantis or a microbe comprising an rRNA sequence with at least about 97% sequence identity to an rRNA from Bifidobacterium infantis. In some embodiments, each pill or capsule comprises at least 1×10⁶ CFU of Clostridium beijerinckii or a microbe comprising an rRNA sequence with at least about 97% sequence identity to an rRNA from Clostridium beijerinckii. In some embodiments, each pill or capsule comprises at least 1×10⁶ CFU of Clostridium butyricum or a microbe comprising an rRNA sequence with at least about 97% sequence identity to an rRNA from Clostridium butyricum. In some embodiments, each pill or capsule comprises between about 1×10⁶ CFU and 1×10¹² CFU of total microbes. In some embodiments, each pill or capsule comprises between about 1×10⁶ CFU and 1×10¹² CFU of the at least one isolated and purified mucin-regulating microbe. In some embodiments, each pill or capsule comprises between about 1×10⁶ CFU and 1×10¹² CFU of the at least one isolated and purified butyrate-producing microbe. In some embodiments, each pill or capsule comprises between about 1×10⁶ CFU and 1×10¹² CFU of Akkermansia muciniphila, a microbe comprising an rRNA sequence with at least about 97% sequence identity to an rRNA from Akkermansia muciniphila, or a microbe comprising an rRNA sequence with at least about 97% sequence identity to any one of SEQ ID NOS: 1-6. In some embodiments, each pill or capsule comprises between about 1×10⁶ CFU and 1×10¹² CFU of Eubacterium hallii or a microbe comprising an rRNA sequence with at least about 97% sequence identity to an rRNA from Eubacterium hallii. In some embodiments, each pill or capsule comprises between about 1×10⁶ CFU and 1×10¹² CFU of Bifidobacterium infantis or a microbe comprising an rRNA sequence with at least about 97% sequence identity to an rRNA from Bifidobacterium infantis. In some embodiments, each pill or capsule comprises between about 1×10⁶ CFU and 1×10¹² CFU of Clostridium beijerinckii or a microbe comprising an rRNA sequence with at least about 97% sequence identity to an rRNA from Clostridium beijerinckii. In some embodiments, each pill or capsule comprises between about 1×10⁶ CFU and 1×10¹² CFU of Clostridium butyricum or a microbe comprising an rRNA sequence with at least about 97% sequence identity to an rRNA from Clostridium butyricum. In some embodiments, one dose of the composition comprises at least one of the one pills or capsules. In some embodiments, one dose of the composition comprises at least two of the pills or capsules. In some embodiments, one dose of the composition comprises one to six of the pills or capsules. In some embodiments, the composition is administered to the subject at least weekly. In some embodiments, the composition is administered to the subject at least daily. In some embodiments, the composition is administered to the subject at least twice a day. In some embodiments, each dose of the composition comprises at least 1×10⁶ CFU of total microbes. In some embodiments, each dose of the composition comprises at least 1×10⁶ CFU of the at least one isolated and purified mucin-regulating microbe. In some embodiments, each dose of the composition comprises at least 1×10⁶ CFU of the at least one isolated and purified butyrate-producing microbe. In some embodiments, each dose of the composition comprises at least 1×10⁶ CFU of Akkermansia muciniphila, a microbe comprising an rRNA sequence with at least about 97% sequence identity to an rRNA from Akkermansia muciniphila, or a microbe comprising an rRNA sequence with at least about 97% sequence identity to any one of SEQ ID NOS: 1-6. In some embodiments, each dose of the composition comprises at least 1×10⁶ CFU of Eubacterium hallii or a microbe comprising an rRNA sequence with at least about 97% sequence identity to an rRNA from Eubacterium hallii. In some embodiments, each dose of the composition comprises at least 1×10⁶ CFU of Bifidobacterium infantis or a microbe comprising an rRNA sequence with at least about 97% sequence identity to an rRNA from Bifidobacterium infantis. In some embodiments, each dose of the composition comprises at least 1×10⁶ CFU of Clostridium beijerinckii or a microbe comprising an rRNA sequence with at least about 97% sequence identity to an rRNA from Clostridium beijerinckii. In some embodiments, each dose of the composition comprises at least 1×10⁶ CFU of Clostridium butyricum or a microbe comprising an rRNA sequence with at least about 97% sequence identity to an rRNA from Clostridium butyricum. In some embodiments, each dose of the composition comprises between about 1×10⁶ CFU and 1×10¹² CFU of total microbes. In some embodiments, each dose of the composition comprises between about 1×10⁶ CFU and 1×10¹² CFU of the at least one isolated and purified mucin-regulating microbe. In some embodiments, each dose of the composition comprises between about 1×10⁶ CFU and 1×10¹² CFU of the at least one isolated and purified butyrate-producing microbe. In some embodiments, each dose of the composition comprises between about 1×10⁶ CFU and 1×10¹² CFU of Akkermansia muciniphila, a microbe comprising an rRNA sequence with at least about 97% sequence identity to an rRNA from Akkermansia muciniphila, or a microbe comprising an rRNA sequence with at least about 97% sequence identity to any one of SEQ ID NOS: 1-6. In some embodiments, each dose of the composition comprises between about 1×10⁶ CFU and 1×10¹² CFU of Eubacterium hallii or a microbe comprising an rRNA sequence with at least about 97% sequence identity to an rRNA from Eubacterium hallii. In some embodiments, each dose of the composition comprises between about 1×10⁶ CFU and 1×10¹² CFU of Bifidobacterium infantis or a microbe comprising an rRNA sequence with at least about 97% sequence identity to an rRNA from Bifidobacterium infantis. In some embodiments, each dose of the composition comprises between about 1×10⁶ CFU and 1×10¹² CFU of Clostridium beijerinckii or a microbe comprising an rRNA sequence with at least about 97% sequence identity to an rRNA from Clostridium beijerinckii. In some embodiments, each dose of the composition comprises between about 1×10⁶ CFU and 1×10¹² CFU of Clostridium butyricum or a microbe comprising an rRNA sequence with at least about 97% sequence identity to an rRNA from Clostridium butyricum. In some embodiments, insulin sensitivity is increased in the subject. In some embodiments, blood glucose levels are stabilized in the subject. In some embodiments, metabolic syndrome is treated in the subject. In some embodiments, insulin resistance is treated in the subject.

Disclosed herein, in some aspects, is a method of treating a subject with an elevated hemoglobin A1C (hA1C) level, comprising orally administering to the subject a composition comprising Clostridium beijerinckii, Clostridium butyricum, Bifidobacterium infantis, Akkermansia muciniphila, and Eubacterium hallii, thereby reducing the hA1C level in the subject by at least 0.2% of total hemoglobin, wherein the composition is in a form of a pill or a capsule comprising an enteric coating designed to release the contents of the pill or capsule in an ileum of the subject, a colon of the subject, or a combination thereof, wherein the subject is human.

Disclosed herein, in some aspects, is a method of treating prediabetes in a subject, comprising orally administering to the subject a composition comprising Clostridium beijerinckii, Clostridium butyricum, Bifidobacterium infantis, Akkermansia muciniphila, and Eubacterium hallii, thereby treating the prediabetes in the subject, wherein the composition is in a form of a pill or a capsule comprising an enteric coating designed to release the contents of the pill or capsule in an ileum of the subject, a colon of the subject, or a combination thereof, wherein the subject is human.

Disclosed herein, in some aspects, is a method of treating a subject with an elevated hemoglobin A1C (hA1C) level, comprising orally administering to the subject a composition comprising Clostridium beijerinckii, Clostridium butyricum, Bifidobacterium infantis, Akkermansia muciniphila, and Eubacterium hallii, thereby reducing the hA1C level in the subject by at least 0.2% of total hemoglobin, wherein the composition is a dietary supplement, wherein the subject is human.

In some embodiments, the dietary supplement is in a form of a food bar. In some embodiments, the dietary supplement is in a form of a powder. In some embodiments, the dietary supplement is in a form of a liquid.

Disclosed herein, in some aspects, is a method of treating prediabetes in a subject, comprising orally administering to the subject a composition comprising Clostridium beijerinckii, Clostridium butyricum, Bifidobacterium infantis, Akkermansia muciniphila, and Eubacterium hallii, thereby treating the prediabetes in the subject, wherein the composition is a dietary supplement, wherein the subject is human.

In some embodiments, the dietary supplement is in a form of a food bar. In some embodiments, the dietary supplement is in a form of a powder. In some embodiments, the dietary supplement is in a form of a liquid.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates the relative change in levels of hemoglobin A1C (“hA1C”), which can be a measure of long term glucose control, in subjects receiving placebo treatment and microbe compositions over the treatment period as described herein.

FIG. 2 illustrates the relative change in glucose area under the curve (“AUC”) after a meal tolerance test (“MTT”) in subjects receiving placebo treatment and microbe compositions over the treatment period as described herein.

FIGS. 3A-D illustrates, for a group of subjects administered a microbe composition as described herein with or without sulfonylurea, the relative change compared to a placebo group in glucose area under the curve after a meal tolerance test for all patients (FIG. 3A) and for patients not administered sulfonylurea (FIG. 3B), as well as the relative change compared to a placebo group in hemoglobin A1C for all patients (FIG. 3C) and for patients not administered sulfonylurea (FIG. 3D) over the treatment period.

FIG. 4 illustrates the phases of a placebo-controlled, double-blinded, randomized crossover trial. Subjects were randomly distributed into two groups. After a baseline period of three days, one group began a two week treatment phase and the other group began a two week placebo phase. During the placebo phase, subjects were administered a colloidal silicon dioxide placebo twice per day. During the treatment phase, subjects were administered a composition twice per day of isolated and purified microbes that contained a prebiotic, a mucin-regulating microbe, and at least one butyrate-producing microbe. After the two week treatment or placebo phase, both groups went through a three day “washout” phase, with no placebo or treatment composition administered. Following the washout phase, the placebo/treatment phases were “crossed over” the group that had previously undergone a treatment phase began a placebo phase, and the group that had previously undergone a placebo phase began a treatment phase. Subjects underwent a meal tolerance test (MTT) at the beginning and end of each placebo/treatment phase.

FIG. 5 provides an example of data collected by continuous glucose monitoring (CGM).

FIG. 6 provides an example of a subject logging their food, drink, and activity during continuous glucose monitoring (CGM).

FIG. 7 provides an example of data from a subject logging their meal tolerance test (MTT) during continuous glucose monitoring (CGM).

FIG. 8 provides glucose concentration curves for six subjects undergoing a meal tolerance test (MTT) at the beginning of a treatment phase (i.e., before receiving the composition of isolated and purified microbes).

FIG. 9 provides glucose concentration curves for five subjects undergoing a meal tolerance test (MTT) at the end of a treatment phase (i.e., after receiving the composition of isolated and purified microbes), superimposed on the glucose concentration curves from the beginning of the treatment phase.

FIG. 10 illustrates the difference in AUC between the placebo phase and the treatment phase for each subject, calculated using the formula ΔΔAUC=ΔAUC_{TREATMENT}−ΔAUC_{PLACEBO}. A negative ΔΔAUC value indicates that treatment resulted in improved blood glucose control compared to placebo.

FIG. 11A illustrates a strategy to alter short chain fatty acid (SCFA) metabolism in a subject. Microbes in the colon can convert dietary fiber into butyrate, which can have beneficial downstream effects, for example, by altering G-protein coupled receptor (GPCR) signaling, altering GLP-1 secretion, increasing insulin sensitivity, decreasing appetite, or a combination thereof. Compositions and methods of the disclosure can be used to alter a microbiome in a subject to promote butyrate production. For example, a microbiome in a subject can be modified to comprise increased levels of one or more primary fermenter microbes that can convert a prebiotic into a butyrate intermediate (e.g., an intermediate that can serve as a substrate for butyrate production, such as acetate), and to comprise increased levels of one or more secondary fermenter microbes that can convert the butyrate intermediate into butyrate.

FIG. 11B illustrates levels of short-chain fatty acids acetate and butyrate produced by microbes of the disclosure. Microbes A-D primarily produced acetate, which can be a butyrate intermediate (e.g., serve as a substrate for butyrate production by a butyrate-producing microbe). Microbes E, F, and G primarily produced butyrate.

FIG. 12 depicts an example data set from an oral glucose tolerance test (OGTT) in a mouse diet-induced obesity model. Mice administered a composition of the disclosure exhibited significantly lower blood glucose levels during the OGTT than control mice.

FIG. 13 illustrates the abundance of microbes in stool samples. Stool samples were collected from human subjects before they commenced taking pills comprising isolated and purified microbes of the disclosure, while they were taking the pills (day 7—low dose; and day 14—high dose), and after a 14 day washout period in which they ceased taking the pills.

FIG. 14 provides an example of how compositions and methods of the disclosure can be used to alter a microbiome in a subject to elicit health benefits. A composition of the disclosure can comprise a combination of microbes for producing butyrate in a subject. For example, the combination can comprise one or more primary fermenter microbes and/or mucin-regulating microbes that can produce a butyrate intermediate (e.g. lactate, acetate, mucin-derived sugars) when provided with an energy source or prebiotic (e.g. fiber). A composition of the disclosure can comprise one or more secondary fermenter microbes and/or butyrate-producing microbes that can convert the butyrate intermediate into butyrate.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Described herein are methods for treating type 2 diabetes or prediabetes, comprising administering to a subject a composition comprising one or more butyrate-producing microbes and one or more mucin-regulating microbes. Such a composition can reduce hemoglobin AlC levels in a subject. Some compositions disclosed herein can reduce the glucose Area Under the Curve (AUC) for a subject after a meal tolerance test (MTT). In some embodiments, a composition disclosed herein can reduce fasting glucose levels in a subject.

Altering the microbiome to treat various disorders and improve well-being is an area of great interest and inquiry. Prediabetes and type 2 diabetes are examples of disorders which can affect a large proportion of the population. Prediabetes and type 2 diabetes can be associated with significant health problems. In some cases, prediabetes and type 2 diabetes are not well controlled, or cannot be well controlled. Thus, it may be of interest to alter the microbiome in order to manage, control, treat, or cure type 2 diabetes.

Existing methods of treating diabetes include the use of thiazolidinediones, gliptins, GLP-1 agonists, SLGT2 inhibitors, dipeptidyl peptidase 4 inhibitors, insulin therapy, metformin, sulfonylurea, diet, and exercise. However, these methods have been associated with certain undesirable side effects, including poor compliance, hypoglycemia, increased risk of cardiovascular disease, pancreatitis, ketoacidosis, lower extremity amputations, diarrhea, anemia, nausea, atrophy, allergies, atherosclerosis, and increased risk of bone fractures. Compositions disclosed herein and methods for using the same are capable of yielding comparable therapeutic results by at least some measures without such undesirable side effects. Moreover, compositions disclosed herein may even modulate, or enhance, therapeutic effects of certain therapeutic agents, e.g., metformin, when co-administered.

Described herein are probiotic compositions and methods of using the probiotic compositions to prevent, manage, control, treat, or cure prediabetes and/or type 2 diabetes. In some cases, a composition can be therapeutic. Some probiotic compositions can alter the microbiome temporarily. Some probiotic compositions can alter the microbiome permanently or for an extended period of time (e.g., microbes administered in a composition may continue to live and grow in a subject's intestinal microbiome even after the composition is no longer administered). In some cases, the altering of the microbiome can provide a therapeutic effect for subjects having prediabetes and/or subjects having type 2 diabetes. In some cases, therapeutic effect can include increased insulin secretion. In some cases, therapeutic effect can include decreased insulin resistance. A therapeutic effect may be shown, for example, by tests indicating a reduced fasting glucose level, a reduced hemoglobin A1C level, a reduced post prandial glucose level, or a reduced glucose area under the curve (AUC) after a meal tolerance test (MTT). If a patient is currently on insulin therapy, a therapeutic effect may include a reduced or eliminated need for insulin.

The terms “microbes” and “microorganisms” can be used interchangeably herein and can refer to bacteria, archaea, eukaryotes (e.g. protozoa, fungi, yeast), and viruses, including bacterial viruses (i.e. phage).

The term “microbiome”, “microbiota”, and “microbial habitat” are used interchangeably herein and can refer to the ecological community of microorganisms that live on or in a subject's body. The microbiome can be comprised of commensal, symbiotic, and/or pathogenic microorganisms. Microbiomes can exist on or in many, or most parts of the subject. Some non-limiting examples of habitats of microbiome can include: body surfaces, body cavities, body fluids, the gut, the colon, skin surfaces and pores, vaginal cavity, umbilical regions, conjunctival regions, intestinal regions, the stomach, the nasal cavities and passages, the gastrointestinal tract, the urogenital tracts, saliva, mucus, and feces.

The term “prebiotic” as used herein can be a general term to refer to chemicals and/or ingredients that can affect the growth and/or activity of microorganisms in a host (e.g. can allow for specific changes in the composition and/or activity in the microbiome). Prebiotics can confer a health benefit on the host. Prebiotics can be selectively fermented, e.g. in the colon. Some non-limiting examples of prebiotics can include: complex carbohydrates, complex sugars, resistant dextrins, resistant starch, amino acids, peptides, nutritional compounds, biotin, polydextrose, oligosaccharides, polysaccharide, fructooligosaccharide (FOS), fructans, soluble fiber, insoluble fiber, fiber, starch, galactooligosaccharides (GOS), inulin, lignin, psyllium, chitin, chitosan, gums (e.g. guar gum), high amylose cornstarch (HAS), cellulose, β-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), locust bean gum, P-glucan, and methylcellulose. Prebiotics can be found in foods (e.g. acacia gum, guar seeds, brown rice, rice bran, barley hulls, chicory root, Jerusalem artichoke, dandelion greens, garlic, leek, onion, asparagus, wheat bran, oat bran, baked beans, whole wheat flour, banana), and breast milk. Prebiotics can also be administered in other forms (e.g. as part of a capsule or dietary supplement). Prebiotics can be administered part of a composition comprising microbes (e.g., probiotics). Prebiotics can be co-administered with a probiotic, or can be administered separately to a probiotic.

The term “probiotic” as used herein can mean one or more microorganisms which, when administered appropriately, can confer a health benefit on the host or subject. Some non-limiting examples microorganisms can be one or more isolated and purified microorganisms selected from the group consisting of Akkermansia muciniphila, Anaerostipes caccae, Bacteroides stercoris, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Collinsella aerofaciens, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus faecis, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburiafaeccis, Roseburia hominis, Roseburia intestinalis, Lacatobacillus bifidus, Lactobacillus johnsonii, Lactobacilli, Acidaminococcus fermentans, Acidaminococcus intestine, Blautia hydrogenotrophica, Citrobacter amalonaticus, Citrobacter freundii, Clostridium aminobutyricum Clostridium bartlettii, Clostridium cochlearium, Clostridium kluyveri, Clostridium limosum, Clostridium malenominatum, Clostridium pasteurianum, Clostridium peptidivorans, Clostridium saccharobutylicum, Clostridium sporosphaeroides, Clostridium sticklandii, Clostridium subterminale, Clostridium symbiosum, Clostridium tetanomorphum, Eubacterium oxidoreducens, Eubacterium pyruvativorans, Methanobrevibacter smithii, Morganella morganii, Peptoniphilus asaccharolyticus, Peptostreptococcus, and any combination thereof.

Administration of microbial or probiotic compositions (e.g., probiotics) to a subject (e.g., to the intestinal tract) may provide many therapeutic benefits. For example, an intestinal microbiota may protect against disease by maintaining a healthy gastrointestinal (GI) tract. Administration of a microbial composition can be considered a natural non-invasive method, for example, to treat a disorder and/or subdue pathogens. Probiotics can be administered orally alone, with food, in food, alongside pharmaceuticals, or any combination thereof.

### Compositions

A “probiotic composition” (also referred to herein as a “microbial composition” or “composition”) described herein can comprise microbes. Microbes in the composition can comprise one or more butyrate-producing microbes and one or more mucin-regulating microbes

A probiotic composition of the disclosure can comprise 2 microbes. A probiotic composition of the disclosure can comprise 3 microbes. A probiotic composition of the disclosure can comprise 4 microbes. Some probiotic compositions can comprise 3-5 microbes. Some probiotic compositions can comprise more than 5 microbes. In some embodiments, a probiotic composition of the disclosure comprises 5 microbes.

A composition as described herein can comprise at least one isolated and purified butyrate-producing microbe and at least one isolated and purified mucin-regulating microbe. In some cases, a composition of the disclosure comprises at least one microbial population that is cultured from an isolated and purified microbe, so as to produce a substantially homogeneous population of the particular microbial species. For ease of discussion, such populations can also generally be referred to herein as being isolated and purified microbes or populations of isolated and purified microbes. Also, as used herein, a composition may comprise mixtures of such populations where the populations had previously been isolated and purified as set forth herein.

In an example, composition of the disclosure can comprise one or more isolated and purified microorganisms selected from the group consisting of Akkermansia muciniphila, Anaerostipes caccae, Bacteroides stercoris, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Collinsella aerofaciens, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus faecis, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburiafaeccis, Roseburia hominis, Roseburia intestinalis, Lacatobacillus biidus, Lactobacillus johnsonii, Lactobacilli, Acidaminococcus fermentans, Acidaminococcus intestine, Blautia hydrogenotrophica, Citrobacter amalonaticus, Citrobacter freundii, Clostridium aminobutyricum Clostridium bartlettii, Clostridium cochlearium, Clostridium kluyveri, Clostridium limosum, Clostridium malenominatum, Clostridium pasteurianum, Clostridium peptidivorans, Clostridium saccharobutylicum, Clostridium sporosphaeroides, Clostridium sticklandii, Clostridium subterminale, Clostridium symbiosum, Clostridium tetanomorphum, Eubacterium oxidoreducens, Eubacterium pyruvativorans, Methanobrevibacter smithii, Morganella morganii, Peptoniphilus asaccharolyticus, and Peptostreptococcus.

In some embodiments, compositions and methods of the disclosure can be used to alter a microbiome in a subject to promote butyrate production. Butyrate production can be useful for the treatment of disorders disclosed herein (for example, prediabetes and type 2 diabetes). Butyrate can exhibit beneficial metabolic and epigenetic effects in a subject. Butyrate can exhibit beneficial downstream effects, for example, by altering G-protein coupled receptor (GPCR) signaling, altering GLP-1 secretion, increasing insulin sensitivity, decreasing appetite, increasing satiety, decreasing lipogenesis in the liver, increasing fat oxidation in muscle, increasing intestinal gluconeogenesis, decreasing inflammation, improving intestinal integrity or barrier function, decreasing LPS-triggered inflammation, inhibiting histone deacetylases, or a combination thereof.

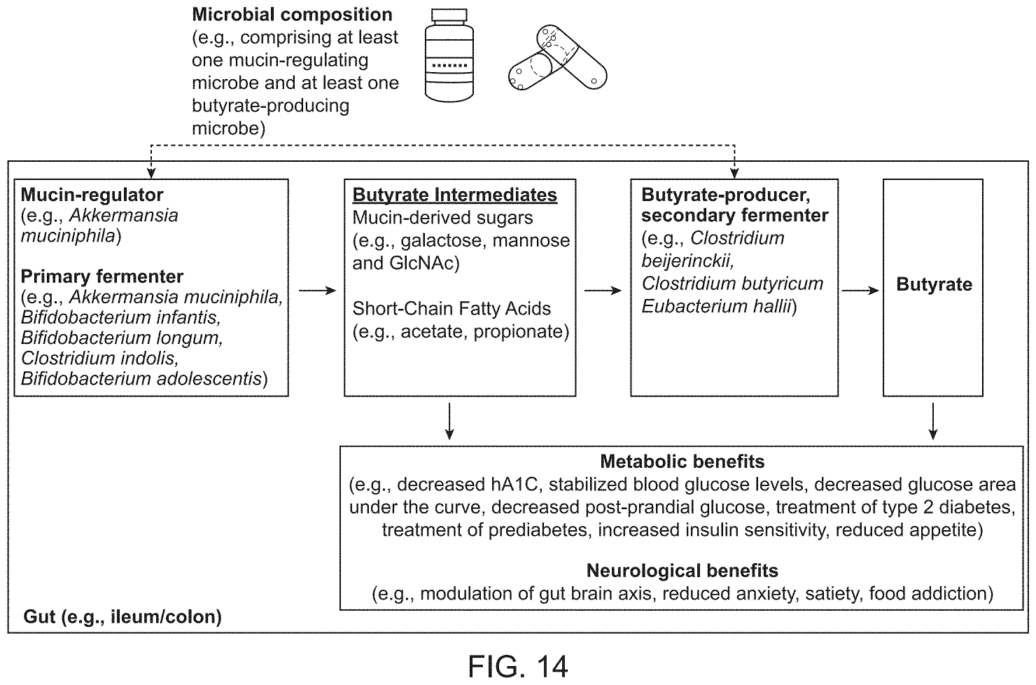

Compositions and methods of the disclosure can be used to alter a microbiome in a subject to elicit health benefits as illustrated in FIG. 14.

For example, a microbiome in a subject can be modified to comprise increased levels of one or more primary fermenter microbes and/or mucin-regulating microbes that can convert mucin or a prebiotic into a butyrate intermediate (e.g., an intermediate that can serve as a substrate for butyrate production, such as acetate), and to comprise increased levels of one or more secondary fermenter microbes and/or butyrate-producing microbes that can convert the butyrate intermediate into butyrate. A primary fermenter microbe can comprise a mucin-regulating microbe.

A mucin-regulating microbe can be a microbe with mucolytic activity, for example, a mucin degrading microbe. A mucin-regulating or mucin-degrading microbe can be capable of growth in a culture medium comprising mucin as a primary energy source. A mucin-degrading microbe (also referred to as a “mucin-degrader”) can degrade mucin (for example, mucin of a host subject) to produce a butyrate intermediate (e.g., sugars) that can be utilized as an energy source by a butyrate-producing microbe. Additionally, the mucin-degrader can produce short-chain fatty acids that can be used as a substrate for butyrate-production by the butyrate producer (e.g., a short chain fatty acid butyrate intermediate, such as acetate). A mucin-regulating microbe can be a primary fermenter. A mucin-regulating microbe can contribute to the downstream production of butyrate to confer a health benefit on a subject. A mucin-regulating microbe can contribute to the growth and/or maintenance of a butyrate-producing microbe in a subject, for example, by degrading mucin to provide a favorable environment for the butyrate-producing microbe, and/or by providing metabolic substrates (e.g., butyrate intermediates) that support growth of the butyrate-producing microbe. Examples of mucin-regulating microbes can include, for example, Akkermansia muciniphila. Table 1 provides 16S rRNA consensus sequence for six illustrative Akkermansia muciniphila strains.

TABLE 1
Illustrative mucin-degrading microbes

| SEQ | | |
|---|---|---|
| ID | | |
| NO. | Strain | Sequence |
| 1 | Akkermansia | AAAATTAATTTGATGGAGAGTTTGATTCTGGCTCAGAACG |
| | muciniphila | AACGCTGGCGGCGTGGATAAGACATGCAAGTCGAACGAG |
| | Strain 1 | AGAATTGCTAGCTTGCTAATAATTCTCTAGTGGCGCACGG |
| | | GTGAGTAACACGTGAGTAACCTGCCCCCGAGAGCGGGAT |
| | | AGCCCTGGGAAACTGGGATTAATACCGCATAGTATCGAA |
| | | AGATTAAAGCAGCAATGCGCTTGGGGATGGGCTCGCGGC |
| | | CTATTAGTTAGTTGGTGAGGTAACGGCTCACCAAGGCGAT |
| | | GACGGGTAGCCGGTCTGAGAGGATGTCCGGCCACACTGG |
| | | AACTGAGACACGGTCCAGACACCTACGGGTGGCAGCAGT |
| | | CGAGAATCATTCACAATGGGGGAAACCCTGATGGTGTGA |
| | | CGCCGCGTGGGGGAATGAAGGTCTTCGGATTGTAAACCCC |
| | | TGTCATGTGGGAGCAAATTAAAAAGATAGTACCACAAGA |
| | | GGAAGAGACGGCTAACTCTGTGCCAGCAGCCGCGGTAAT |
| | | ACAGAGGTCTCAAGCGTTGTTCGGAATCACTGGGCGTAAA |
| | | GCGTGCGTAGGCTGTTTCGTAAGTCGTGTGTGAAAGGCGC |
| | | GGGCTCAACCCGCGGACGGCACATGATACTGCGAGACTA |
| | | GAGTAATGGAGGGGGAACCGGAATTCTCGGTGTAGCAGT |
| | | GAAATGCGTAGATATCGAGAGGAACACTCGTGGCGAAGG |
| | | CGGGTTCCTGGACATTAACTGACGCTGAGGCACGAAGGCC |
| | | AGGGGAGCGAAAGGGATTAGATACCCCTGTAGTCCTGGC |
| | | AGTAAACGGTGCACGCTTGGTGTGCGGGGAATCGACCCCC |
| | | TGCGTGCCGGAGCTAACGCGTTAAGCGTGCCGCCTGGGGA |
| | | GTACGGTCGCAAGATTAAAACTCAAAGAAATTGACGGGG |
| | | ACCCGCACAAGCGGTGGAGTATGTGGCTTAATTCGATGCA |
| | | ACGCGAAGAACCTTACCTGGGCTTGACATGTAATGAACAA |
| | | CATGTGAAAGCATGCGACTCTTCGGAGGCGTTACACAGGT |
| | | GCTGCATGGCCGTCGTCAGCTCGTGTCGTGAGATGTTTGG |
| | | TTAAGTCCAGCAACGAGCGCAACCCCTGTTGCCAGTTACC |
| | | AGCACGTGAAGGTGGGGACTCTGGCGAGACTGCCCAGAT |
| | | CAACTGGGAGGAAGGTGGGGACGACGTCAGGTCAGTATG |
| | | GCCCTTATGCCCAGGGCTGCACACGTACTACAATGCCCAG |
| | | TACAGAGGGGGCCGAAGCCGCGAGGCGGAGGAAATCCTG |
| | | AAAACTGGGCCCAGTTCGGACTGTAGGCTGCAACCCGCCT |
| | | ACACGAAGCCGGAATCGCTAGTAATGGCGCATCAGCTAC |
| | | GGCGCCGTGAATACGTTCCCGGGTCTTGTACACACCGCCC |
| | | GTCACATCATGGAAGCCGGTCGCACCCGAAGTATCTGAAG |
| | | CCAACCGCAAGGAGGCAGGGTCCTAAGGTGAGACTGGTA |
| | | ACTGGGATGAAGTCGTAACAAGGTAGCCGTAGGGGAACC |
| | | TGCGGCTGGATCACCTCCTTTCT |
| 2 | Akkermansia | AGAGTTTGATTCTGGCTCAGAACGAACGCTGGCGGCGTGG |
| | muciniphila | ATAAGACATGCAAGTCGAACGAGAGAATTGCTAGCTTGCT |
| | Strain 2 | AATAATTCTCTAGTGGCGCACGGGTGAGTAACACGTGAGT |
| | | AACCTGCCCCCGAGAGCGGGATAGCCCTGGGAAACTGGG |
| | | ATTAATACCGCATAGTATCGCAAGATTAAAGCAGCAATGC |
| | | GCTTGGGGATGGGCTCGCGGCCTATTAGTTAGTTGGTGAG |
| | | GTAACGGCTCACCAAGGCGATGACGGGTAGCCGGTCTGA |
| | | GAGGATGTCCGGCCACACTGGAACTGAGACACGGTCCAG |
| | | ACACCTACGGGTGGCAGCAGTCGAGAATCATTCACAATG |
| | | GGGGAAACCCTGATGGTGCGACGCCGCGTGGGGGAATGA |
| | | AGGTCTTCGGATTGTAAACCCCTGTCATGTGGGAGCAAAT |
| | | TAAAAAGATAGTACCACAAGAGGAAGAGACGGCTAACTC |
| | | TGTGCCAGCAGCCGCGGTAATACAGAGGTCTCAAGCGTTG |
| | | TTCGGAATCACTGGGCGTAAAGCGTGCGTAGGCTGTTTCG |
| | | TAAGTCGTGTGTGAAAGGCGCGGGCTCAACCCGCGGACG |
| | | GCACATGATACTGCGAGACTAGAGTAATGGAGGGGGAAC |
| | | CGGAATTCTCGGTGTAGCAGTGAAATGCGTAGATATCGAG |
| | | AGGAACACTCGTGGCGAAGGCGGGTTCCTGGACATTAACT |
| | | GACGCTGAGGCACGAAGGCCAGGGGAGCGAAAGGGATTA |
| | | GATACCCCTGTAGTCCTGGCAGTAAACGGTGCACGCTTGG |
| | | TGTGCGGGGAATCGACCCCCTGCGTGCCGGAGCTAACGCG |
| | | TTAAGCGTGCCGCCTGGGGAGTACGGTCGCAAGATTAAA |
| | | ACTCAAAGAAATTGACGGGGACCCGCACAAGCGGTGGAG |
| | | TATGTGGCTTAATTCGATGCAACGCGAAGAACCTTACCTG |
| | | GGCTTGACATGTAATGAACAACATGTGAAAGCATGCGACT |
| | | CTTCGGAGGCGTTACACAGGTGCTGCATGGCCGTCGTCAG |
| | | CTCGTGTCGTGAGATGTTTGGTTAAGTCCAGCAACGAGCG |
| | | CAACCCCTGTTGCCAGTTACCAGCACGTGAAGGTGGGGAC |
| | | TCTGGCGAGACTGCCCAGATCAACTGGGAGGAAGGTGGG |
| | | GACGACGTCAGGTCAGTATGGCCCTTATGCCCAGGGCTGC |
| | | ACACGTACTACAATGCCCAGTACAGAGGGGGCCGAAGCC |
| | | GCGAGGCGGAGGAAATCCTAAAAACTGGGCCCAGTTCGG |
| | | ACTGTAGGCTGCAACCCGCCTACACGAAGCCGGAATCGCT |
| | | AGTAATGGCGCATCAGCTACGGCGCCGTGAATACGTTCCC |
| | | GGGTCTTGTACACACCGCCCGTCACATCATGGAAGCCGGT |
| | | CGCACCCGAAGTATCTGAAGCCAACCGCAAGGAGGCAGG |
| | | GTCCTAAGGTGAGACTGGTAACTGGGATGAAGTCGTAAC |
| | | AAGGTAGCCGTAGGGGAACCTGCGGCTGGATCACCTCCTT |
| | | TCT |
| 3 | Akkermansia | CTGGCGGCGTGGATAAGACATGCAAGTCGAACGAGAGAA |
| | muciniphila | TTGCTAGCTTGCTAATAATTCTCTAGTGGCGCACGGGTGA |
| | Strain 3 | GTAACACGTGAGTAACCTGCCCCCGAGAGCGGGATAGCC |
| | | CTGGGAAACTGGGATTAATACCGCATAGTATCGAAAGATT |
| | | AAAGCAGCAATGCGCTTGGGGATGGGCTCGCGGCCTATTA |
| | | GTTAGTTGGTGAGGTAACGGCTCACCAAGGCGATGACGG |
| | | GTAGCCGGTCTGAGAGGATGTCCGGCCACACTGGAACTG |
| | | AGACACGGTCCAGACACCTACGGGTGGCAGCAGTCGAGA |
| | | ATCATTCACAATGGGGGAAACCCTGATGGTGCGACGCCGC |
| | | GTGGGGGAATGAAGGTCTTCGGATTGTAAACCCCTGTCAT |
| | | GTGGGAGCAAATTAAAAAGATAGTACCACAAGAGGAAGA |
| | | GACGGCTAACTCTGTGCCAGCAGCCGCGGTAATACAGAG |
| | | GTCTCAAGCGTTGTTCGGAATCACTGGGCGTAAAGCGTGC |
| | | GTAGGCTGTTTCGTAAGTCGTGTGTGAAAGGCGCGGGCTC |
| | | AACCCGCGGACGGCACATGATACTGCGAGACTAGAGTAA |
| | | TGGAGGGGGAACCGGAATTCTCGGTGTAGCAGTGAAATG |
| | | CGTAGATATCGAGAGGAACACTCGTGGCGAAGGCGGGTT |
| | | CCTGGACATTAACTGACGCTGAGGCACGAAGGCCAGGGG |
| | | AGCGAAAGGGATTAGATACCCCTGTAGTCCTGGCAGTAA |
| | | ACGGTGCACGCTTGGTGTGCGGGGAATCGACCCCCTGCGT |
| | | GCCGGAGCTAACGCGTTAAGCGTGCCGCCTGGGGAGTAC |
| | | GGTCGCAAGATTAAAACTCAAAGAAATTGACGGGGACCC |
| | | GCACAAGCGGTGGAGTATGTGGCTTAATTCGATGCAACGC |
| | | GAAGAACCTTACCTGGGCTTGACATGTAATGAACAACATG |
| | | TGAAAGCATGCGACTCTTCGGAGGCGTTACACAGGTGCTG |
| | | CATGGCCGTCGTCAGCTCGTGTCGTGAGATGTTTGGTTAA |
| | | GTCCAGCAACGAGCGCAACCCCTGTTGCCAGTTACCAGCA |
| | | CGTGAAGGTGGGGACTCTGGCGAGACTGCCCAGATCAAC |
| | | TGGGAGGAAGGTGGGGACGACGTCAGGTCAGTATGGCCC |
| | | TTATGCCCAGGGCTGCACACGTACTACAATGCCCAGTACA |
| | | GAGGGGGCCGAAGCCGCGAGGCGGAGGAAATCCTAAAAA |
| | | CTGGGCCCAGTTCGGACTGTAGGCTGCAACCCGCCTACAC |
| | | GAAGCCGGAATCGCTAGTAATGGCGCATCAGCTACGGCG |
| | | CCGTGAATACGTTCCCGGGTCTTGTACACACCGCCCGTCA |
| | | CATCATGGAAGCCGGTCGCACCCGAAGTATCTGAAGCCA |
| | | ACCGCAAGGAGGCAGGGTCCTAAGGTGAGACTGGTAACT |
| | | GGGATGAAGTCGTAACAAGGTAGCCGTAGGGGAACCTGC |
| | | GGCTGGATCACCTCCTTTCTATGGAGCAAGTGCACGGAAG |
| | | TGCAC |
| 4 | Akkermansia | TGCTAGCTTGCTAATAATTCTCTAGTGGCGCACGGGTGAG |
| | muciniphila | TAACACGTGAGTAACCTGCCCCCGAGAGCGGGATAGCCCT |
| | Strain 4 | GGGAAACTGGGATTAATACCGCATAGTATCGCAAGATTA |
| | | AAGCAGCAATGCGCTTGGGGATGGGCTCGCGGCCTATTAG |
| | | TTAGTTGGTGAGGTAACGGCTCACCAAGGCGATGACGGGT |
| | | AGCCGGTCTGAGAGGATGTCCGGCCACACTGGAACTGAG |
| | | ACACGGTCCAGACACCTACGGGTGGCAGCAGTCGAGAAT |
| | | CATTCACAATGGGGGAAACCCTGATGGTGCGACGCCGCGT |
| | | GGGGGAATGAAGGTCTTCGGATTGTAAACCCCTGTCATGT |
| | | GGGAGCAAATTAAAAAGATAGTACCACAAGAGGAAGAGA |
| | | CGGCTAACTCTGTGCCAGCAGCCGCGGTAATACAGAGGTC |
| | | TCAAGCGTTGTTCGGAATCACTGGGCGTAAAGCGTGCGTA |
| | | GGCTGTTTCGTAAGTCGTGTGTGAAAGGCGCGGGCTCAAC |
| | | CCGCGGACGGCACATGATACTGCGAGACTAGAGTAATGG |
| | | AGGGGGAACCGGAATTCTCGGTGTAGCAGTGAAATGCGT |
| | | AGATATCGAGAGGAACACTCGTGGCGAAGGCGGGTTCCT |
| | | GGACATTAACTGACGCTGAGGCACGAAGGCCAGGGGAGC |
| | | GAAAGGGATTAGATACCCCTGTAGTCCTGGCAGTAAACG |
| | | GTGCACGCTTGGTGTGCGGGGAATCGACCCCCTGCGTGCC |
| | | GGAGCTAACGCGTTAAGCGTGCCGCCTGGGGAGTACGGT |
| | | CGCAAGATTAAAACTCAAAGAAATTGACGGGGACCCGCA |
| | | CAAGCGGTGGAGTATGTGGCTTAATTCGATGCAACGCGAA |
| | | GAACCTTACCTGGGCTTGACATGTAATGAACAACATGTGA |
| | | AAGCATGCGACTCTTCGGAGGCGTTACACAGGTGCTGCAT |
| | | GGCCGTCGTCAGCTCGTGTCGTGAGATGTTTGGTTAAGTC |
| | | CAGCAACGAGCGCAACCCCTGTTGCCAGTTACCAGCACGT |
| | | GAAGGTGGGGACTCTGGCGAGACTGCCCAGATCAACTGG |
| | | GAGGAAGGTGGGGACGACGTCAGGTCAGTATGGCCCTTA |
| | | TGCCCAGGGCTGCACACGTACTACAATGCCCAGTACAGAG |
| | | GGGGCCGAAGCCGCGAGGCGGAGGAAATCCTAAAAACTG |
| | | GGCCCAGTTCGGACTGTAGGCTGCAACCCGCCTACACGAA |
| | | GCCGGAATCGCTAGTAATGGCGCATCAGCTACGGCGCCGT |
| | | GAATACGTTCCCGGGTCTTGTACACACCGCCCGTCACATC |
| | | ATGGAAGCCGGTCGCACCCGAAGTATCTGAAGCCAACCG |
| | | CAAGGAGGCAGGGTCCTAAGGTGAGACTGGTAACTGGGA |
| | | TGAAGTCGTAACAAGGTAGCCGTAGGGGAACCTGCGGCT |
| | | GGATCACCTCCTTTC |
| 5 | Akkermansia | TCCAGCAATTTCAAAAATTAATTTGATGGAGAGTTTGATT |
| | muciniphila | CTGGCTCAGAACGAACGCTGGCGGCGTGGATAAGACATG |
| | Strain 5 | CAAGTCGAACGAGAGAATTGCTAGCTTGCTAATAATTCTC |
| | | TAGTGGCGCACGGGTGAGTAACACGTGAGTAACCTGCCCC |
| | | CGAGAGCGGGATAGCCCTGGGAAACTGGGATTAATACCG |
| | | CATAGTATCGCAAGATTAAAGCAGCAATGCGCTTGGGGAT |
| | | GGGCTCGCGGCCTATTAGTTAGTTGGTGAGGTAACGGCTC |
| | | ACCAAGGCGATGACGGGTAGCCGGTCTGAGAGGATGTCC |
| | | GGCCACACTGGAACTGAGACACGGTCCAGACACCTACGG |
| | | GTGGCAGCAGTCGAGAATCATTCACAATGGGGGAAACCC |
| | | TGATGGTGCGACGCCGCGTGGGGGAATGAAGGTCTTCGG |
| | | ATTGTAAACCCCTGTCATGTGGGAGCAAATTAAAAAGATA |
| | | GTACCACAAGAGGAAGAGACGGCTAACTCTGTGCCAGCA |
| | | GCCGCGGTAATACAGAGGTCTCAAGCGTTGTTCGGAATCA |
| | | CTGGGCGTAAAGCGTGCGTAGGCTGTTTCGTAAGTCGTGT |
| | | GTGAAAGGCGCGGGCTCAACCCGCGGACGGCACATGATA |
| | | CTGCGAGACTAGAGTAATGGAGGGGGAACCGGAATTCTC |
| | | GGTGTAGCAGTGAAATGCGTAGATATCGAGAGGAACACT |
| | | CGTGGCGAAGGCGGGTTCCTGGACATTAACTGACGCTGAG |
| | | GCACGAAGGCCAGGGGAGCGAAAGGGATTAGATACCCCT |
| | | GTAGTCCTGGCAGTAAACGGTGCACGCTTGGTGTGCGGGG |
| | | AATCGACCCCCTGCGTGCCGGAGCTAACGCGTTAAGCGTG |
| | | CCGCCTGGGGAGTACGGTCGCAAGATTAAAACTCAAAGA |
| | | AATTGACGGGGACCCGCACAAGCGGTGGAGTATGTGGCT |
| | | TAATTCGATGCAACGCGAAGAACCTTACCTGGGCTTGACA |
| | | TGTAATGAACAACATGTGAAAGCATGCGACTCTTCGGAGG |
| | | CGTTACACAGGTGCTGCATGGCCGTCGTCAGCTCGTGTCG |
| | | TGAGATGTTTGGTTAAGTCCAGCAACGAGCGCAACCCCTG |
| | | TTGCCAGTTACCAGCACGTGAAGGTGGGGACTCTGGCGAG |
| | | ACTGCCCAGATCAACTGGGAGGAAGGTGGGGACGACGTC |
| | | AGGTCAGTATGGCCCTTATGCCCAGGGCTGCACACGTACT |
| | | ACAATGCCCAGTACAGAGGGGGCCGAAGCCGCGAGGCGG |
| | | AGGAAATCCTAAAAACTGGGCCCAGTTCGGACTGTAGGCT |
| | | GCAACCCGCCTACACGAAGCCGGAATCGCTAGTAATGGC |
| | | GCATCAGCTACGGCGCCGTGAATACGTTCCCGGGTCTTGT |
| | | ACACACCGCCCGTCACATCATGGAAGCCGGTCGCACCCGA |
| | | AGTATCTGAAGCCAACCGCAAGGAGGCAGGGTCCTAAGG |
| | | TGAGACTGGTAACTGGGATGAAGTCGTAACAAGGTAGCC |
| | | GTAGGGGAACCTGCGGCTGGATCACCTCCTTTCTNNNNNN |
| | | ATGGAGCAAGTA |
| 6 | Akkermansia | GAGTTTGATTCTGGCTCAGAACGAACGCTGGCGGCGTGGA |
| | muciniphila | TAAGACATGCAAGTCGAACGAGAGAATTGCTAGCTTGCTA |
| | Strain 6 | ATAATTCTCTAGTGGCGCACGGGTGAGTAACACGTGAGTA |
| | | ACCTGCCCCCGAGAGCGGGATAGCCCTGGGAAACTGGGA |
| | | TTAATACCGCATAGTATCGAAAGATTAAAGCAGCAATGCG |
| | | CTTGGGGATGGGCTCGCGGCCTATTAGTTAGTTGGTGAGG |
| | | TAACGGCTCACCAAGGCGATGACGGGTAGCCGGTCTGAG |
| | | AGGATGTCCGGCCACACTGGAACTGAGACACGGTCCAGA |
| | | CACCTACGGGTGGCAGCAGTCGAGAATCATTCACAATGG |
| | | GGGAAACCCTGATGGTGTGACGCCGCGTGGGGGAATGAA |
| | | GGTCTTCGGATTGTAAACCCCTGTCATGTGGGAGCAAATT |
| | | AAAAAGATAGTACCACAAGAGGAAGAGACGGCTAACTCT |
| | | GTGCCAGCAGCCGCGGTAATACAGAGGTCTCAAGCGTTGT |
| | | TCGGAATCACTGGGCGTAAAGCGTGCGTAGGCTGTTTCGT |
| | | AAGTCGTGTGTGAAAGGCGCGGGCTCAACCCGCGGACGG |
| | | CACATGATACTGCGAGACTAGAGTAATGGAGGGGGAACC |
| | | GGAATTCTCGGTGTAGCAGTGAAATGCGTAGATATCGAGA |
| | | GGAACACTCGTGGCGAAGGCGGGTTCCTGGACATTAACTG |
| | | ACGCTGAGGCACGAAGGCCAGGGGAGCGAAAGGGATTAG |
| | | ATACCCCTGTAGTCCTGGCAGTAAACGGTGCACGCTTGGT |
| | | GTGCGGGGAATCGACCCCCTGCGTGCCGGAGCTAACGCGT |
| | | TAAGCGTGCCGCCTGGGGAGTACGGTCGCAAGATTAAAA |
| | | CTCAAAGAAATTGACGGGGACCCGCACAAGCGGTGGAGT |
| | | ATGTGGCTTAATTCGATGCAACGCGAAGAACCTTACCTGG |
| | | GCTTGACATGTAATGAACAACATGTGAAAGCATGCGACTC |
| | | TTCGGAGGCGTTACACAGGTGCTGCATGGCCGTCGTCAGC |
| | | TCGTGTCGTGAGATGTTTGGTTAAGTCCAGCAACGAGCGC |
| | | AACCCCTGTTGCCAGTTACCAGCACGTGAAGGTGGGGACT |
| | | CTGGCGAGACTGCCCAGATCAACTGGGAGGAAGGTGGGG |
| | | ACGACGTCAGGTCAGTATGGCCCTTATGCCCAGGGCTGCA |
| | | CACGTACTACAATGCCCAGTACAGAGGGGGCCGAAGCCG |
| | | CGAGGCGGAGGAAATCCTGAAAACTGGGCCCAGTTCGGA |
| | | CTGTAGGCTGCAACCCGCCTACACGAAGCCGGAATCGCTA |
| | | GTAATGGCGCATCAGCTACGGCGCCGTGAATACGTTCCCG |
| | | GGTCTTGTACACACCGCCCGTCACATCATGGAAGCCGGTC |
| | | GCACCCGAAGTATCTGAAGCCAACCGCAAGGAGGCAGGG |
| | | TCCTAAGGTGAGACTGGTAACTGGGATGAAGTCGTAACA |
| | | AGGTAGCCGTAGGGGAACCTGCGGCTGGATCACCTCCTTT |
| | | CTA |

A composition of the disclosure can comprise a butyrate-producing microbe. A butyrate-producing microbe can be a microbe that can produce butyrate. Non-limiting examples of butyrate-producing microbes include Clostridium beijerinckii, Clostridium butyricum, Eubacterium hallii, and Faecalibacterium prausnitzii.

A composition of the disclosure can comprise a combination of microbes for producing butyrate in a subject, as illustrated In FIG. 14. For example, the combination can comprise a first microbe and a second microbe. The first microbe can produce a butyrate intermediate (e.g. lactate, acetate, mucin-derived sugars) when provided with an energy source or prebiotic (e.g. fiber). The second microbe can convert the butyrate intermediate produced by the first microbe into butyrate (e.g., a butyrate-producing microbe). In some embodiments the first microbe can be a primary fermenter and the second microbe can be a secondary fermenter. Non-limiting examples of microbes that can produce intermediate molecules for butyrate production include Akkermansia muciniphila, Bifidobacterium adolescentis, Bifidobacterium infantis and Bifidobacterium longum. Non-limiting examples of a microbe that can use the intermediate molecules to produce butyrate include Clostridium beijerinckii, Clostridium butyricum, Clostridium indolis, Eubacterium hallii, and Faecalibacterium prausnitzii. A composition can comprise at least one microbe for production of butyrate-intermediate molecules and at least one microbe for conversion of the butyrate intermediate to butyrate. The composition can additionally comprise a substrate for the first microbe that produces the butyrate intermediate.

For example, the composition can comprise a prebiotic as disclosed herein, and/or mucin. In some embodiments, a composition of the disclosure comprises inulin.

One microbe or a combination of microbes for producing butyrate in a subject can comprise a combination of enzymes from one or more metabolic pathways for producing butyrate. One microbe or a combination of microbes for producing butyrate in a subject can produce butyrate via a particular butyrate metabolic pathway. Production of butyrate by a particular metabolic pathway can alter the amount of butyrate produced. Production of butyrate by a particular metabolic pathway can alter the abundance or relative proportion of butyrate intermediates, which in some embodiments can further contribute to a health benefit in a subject (for example, treatment of prediabetes or type 2 diabetes).

Examples of buyrate-producing metabolic pathways include, but are not limited to, the acetyl-CoA butyrate-producing pathway, the glutarate butyrate-producing pathway, the 4-aminobutyrate butyrate-producing pathway, and the lysine butyrate-producing pathway.

The abundance of the acetyl-CoA butyrate-producing pathway is reduced in stool samples from human patients with type 2 diabetes, indicating that compositions or methods that boost this pathway could be useful for preventing, treating, reducing, or delaying progression of prediabetes, type 2 diabetes, and related conditions. In some embodiments, increased abundance of the acetyl-CoA butyrate-producing pathway in a subject can allow for the production of higher levels of butyrate in the subject. Increased abundance of the acetyl-CoA butyrate-producing pathway can alter the abundance or relative proportion of select butyrate intermediates, which can further contribute to a health benefit in a subject (for example, treatment of prediabetes or type 2 diabetes).

Enzymes in the acetyl-CoA butyrate-producing pathway can include thl (acetyl-CoA acetyltransferase/thiolase), bhbd (O-hydroxybutyryl-CoA dehydrogenase), cro (crotonase), but (butyryl-CoA:acetate CoA transferase), buk (butyrate kinase), and bcd-eftAB (butyryl-CoA dehydrogenase including electron transfer protein α, β subunit).

Enzymes in the glutarate butyrate-producing pathway can include gctAB (glutaconate-CoA transferase α, β subunit), hcCoAdABC (2-hydroxyglutaryl-CoA dehydratase α, β, and γ subunit), gcdAB (glutaconyl-CoA decarboxylase α, β subunit), and bcd-eftAB (butyryl-CoA dehydrogenase including electron transfer protein α, β subunit).

Enzymes in the 4-aminobutyrate butyrate-producing pathway can include abfH (4-hydroxybutyrate dehydrogenase), 4hbt (butyryl-CoA:4-hydroxybutyrate CoA transferase), abfD (4-hydroxybutyryl-CoA dehydratase), abfD (vinylacetyl-CoA 3,2-isomerase), and bcd-eftAB (butyryl-CoA dehydrogenase including electron transfer protein α, β subunit).

Enzymes in the lysine butyrate-producing pathway can include kamA (lysine-2,3-aminomutase), kamDE (β-lysine-5,6-aminomutase α, β subunit), kdd (3,5-diaminohexanoate dehydrogenase), kce (3-keto-5-aminohexanoate cleavage enzyme), kal (3-aminobutyryl-CoA ammonia-lyase), atoAD (butyryl-CoA:acetoacetate CoA transferase α, β subunit), and bcd-eftAB (butyryl-CoA dehydrogenase including electron transfer protein α, β subunit).

A composition can comprise a microbe encoding a butyrate kinase enzyme (e.g., EC 2.7.2.7; MetaCyc Reaction ID R11-RXN). Butyrate kinase is an enzyme belonging to a family of transferases, for example those transferring phosphorus-containing groups (e.g., phosphotransferases) with a carboxy group as acceptor. The systematic name of this enzyme class can be ATP:butanoate 1-phosphotransferase. Butyrate kinase can participate in butyrate metabolism. Butyrate kinase can catalyze the following reaction:

ADP+butyryl-phosphate [symbol] ATP+butyrate

A composition of the disclosure can comprise a microbe with a butyrate-Coenzyme A. Butyrate-Coenzyme A, also butyryl-coenzyme A, can be a coenzyme A-activated form of butyric acid. It can be acted upon by butyryl-CoA dehydrogenase and can be an intermediary compound in acetone-butanol-ethanol fermentation. Butyrate-Coenzyme A can be involved in butyrate metabolism.

A composition of the disclosure can comprise a microbe encoding a butyrate-Coenzyme A transferase enzyme. Butyrate-Coenzyme A transferase, also known as butyrate-acetoacetate CoA-transferase, can belong to a family of transferases, for example, the CoA-transferases. The systematic name of this enzyme class can be butanoyl-CoA:acetoacetate CoA-transferase. Other names in common use can include butyryl coenzyme A-acetoacetate coenzyme A-transferase (e.g., EC 2.8.3.9; MetaCyc Reaction ID 2.8.3.9-RXN), and butyryl-CoA-acetoacetate CoA-transferase. Butyrate-Coenzyme A transferase can catalyze the following chemical reaction:

butanoyl-CoA+acetoacetate [symbol] rbutanoate+acetoacetyl-CoA

A composition of the disclosure can comprise a microbe encoding an acetate Coenzyme A transferase (e.g., EC 2.8.3.1/2.8.3.8; MetaCyc Reaction ID BUTYRATE-KINASE-RXN).

A composition of the disclosure can comprise a microbe encoding a Butyryl-Coenzyme A dehydrogenase. Butyryl-CoA dehydrogenase can belong to the family of oxidoreductases, for example, those acting on the CH—CH group of donor with other acceptors. The systematic name of this enzyme class can be butanoyl-CoA:acceptor 2,3-oxidoreductase. Other names in common use can include butyryl dehydrogenase, unsaturated acyl-CoA reductase, ethylene reductase, enoyl-coenzyme A reductase, unsaturated acyl coenzyme A reductase, butyryl coenzyme A dehydrogenase, short-chain acyl CoA dehydrogenase, short-chain acyl-coenzyme A dehydrogenase, 3-hydroxyacyl CoA reductase, and butanoyl-CoA:(acceptor) 2,3-oxidoreductase. Non-limiting examples of metabolic pathways that butyryl-CoA dehydrogenase can participate in include: fatty acid metabolism; valine, leucine and isoleucine degradation; and butanoate metabolism. Butyryl-CoA dehydrogenase can employ one cofactor, FAD. Butyryl-CoA dehydrogenase can catalyze the following reaction:

butyryl-CoA+acceptor [symbol] butenoyl-CoA+reduced acceptor

A composition of the disclosure can comprise a microbe encoding a beta-hydroxybutyryl-CoA dehydrogenase. Beta-hydroxybutyryl-CoA dehydrogenase or 3-hydroxybutyryl-CoA dehydrogenase can belong to a family of oxidoreductases, for example, those acting on the CH—OH group of donor with NAD+ or NADP+ as acceptor. The systematic name of the enzyme class can be (S)-3-hydroxybutanoyl-CoA:NADP+oxidoreductase. Other names in common use can include beta-hydroxybutyryl coenzyme A dehydrogenase, L(+)-3-hydroxybutyryl-CoA dehydrogenase, BHBD, dehydrogenase, L-3-hydroxybutyryl coenzyme A (nicotinamide adenine, dinucleotide phosphate), L-(+)-3-hydroxybutyryl-CoA dehydrogenase, and 3-hydroxybutyryl-CoA dehydrogenase. Beta-hydroxybutyryl-CoA dehydrogenase enzyme can participate in benzoate degradation via co-ligation. Beta-hydroxybutyryl-CoA dehydrogenase enzyme can participate in butanoate metabolism. Beta-hydroxybutyryl-CoA dehydrogenase can catalyze the following reaction:

(S)-3-hydroxybutanoyl-CoA+NADP [symbol] 3-acetoacetyl-CoA+NADPH+H⁺

A composition of the disclosure can comprise a microbe encoding a crotonase. Crotonase can comprise enzymes with, for example, dehalogenase, hydratase, isomerase activities. Crotonase can be implicated in carbon-carbon bond formation, cleavage, and hydrolysis of thioesters. Enzymes in the crotonase superfamily can include, for example, enoyl-CoA hydratase which can catalyse the hydratation of 2-trans-enoyl-CoA into 3-hydroxyacyl-CoA; 3-2trans-enoyl-CoA isomerase or dodecenoyl-CoA isomerise (e.g., EC 5.3.3.8), which can shift the 3-double bond of the intermediates of unsaturated fatty acid oxidation to the 2-trans position; 3-hydroxbutyryl-CoA dehydratase (e.g., crotonase; EC 4.2.1.55), which can be involved in the butyrate/butanol-producing pathway; 4-Chlorobenzoyl-CoA dehalogenase (e.g., EC 3.8.1.6) which can catalyze the conversion of 4-chlorobenzoate-CoA to 4-hydroxybenzoate-CoA; dienoyl-CoA isomerase, which can catalyze the isomerisation of 3-trans,5-cis-dienoyl-CoA to 2-trans,4-trans-dienoyl-CoA; naphthoate synthase (e.g., MenB, or DHNA synthetase; EC 4.1.3.36), which can be involved in the biosynthesis of menaquinone (e.g., vitamin K2); carnitine racemase (e.g., gene caiD), which can catalyze the reversible conversion of crotonobetaine to L-carnitine in Escherichia coli; Methylmalonyl CoA decarboxylase (e.g., MMCD; EC 4.1.1.41); carboxymethylproline synthase (e.g., CarB), which can be involved in carbapenem biosynthesis; 6-oxo camphor hydrolase, which can catalyze the desymmetrization of bicyclic beta-diketones to optically active keto acids; the alpha subunit of fatty acid oxidation complex, a multi-enzyme complex that can catalyze the last three reactions in the fatty acid beta-oxidation cycle; and AUH protein, which can be a bifunctional RNA-binding homologue of enoyl-CoA hydratase.

A composition of the disclosure can comprise a microbe encoding a thiolase. Thiolases, also known as acetyl-coenzyme A acetyltransferases (ACAT), can convert two units of acetyl-CoA to acetoacetyl CoA, for example, in the mevalonate pathway. Thiolases can include, for example, degradative thiolases (e.g., EC 2.3.1.16) and biosynthetic thiolases (e.g., EC 2.3.1.9). 3-ketoacyl-CoA thiolase, also called thiolase I, can be involved in degradative pathways such as fatty acid beta-oxidation. Acetoacetyl-CoA thiolase, also called thiolase II, can be specific for the thiolysis of acetoacetyl-CoA and can be involved in biosynthetic pathways such as poly beta-hydroxybutyric acid synthesis or steroid biogenesis. A thiolase can catalyze the following reaction:

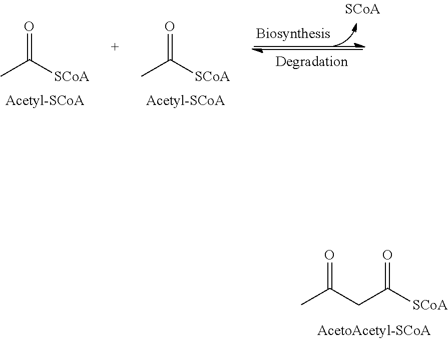

In one non-limiting example, a composition can comprise Bifidobacterium adolescentis and Clostridium indolis. In another illustrative example, a composition can comprise Akkermansia muciniphila, Bifidobacterium infantis, Clostridium beijerinckii, Clostridium butyricum, and, Eubacterium hallii. In another non-limiting example, a composition can comprise Bifidobacterium longum, and Faecalibacterium prausnitzii. In another non-limiting example, a composition can comprise Bifidobacterium infantis, Clostridium beijerinckii, and Clostridium butyricum. In another non-limiting example, a composition can comprise Bifidobacterium infantis, Clostridium beijerinckii, Clostridium butyricum, and Akkermansia muciniphila. In another non-limiting example, a composition can comprise Clostridium beijerinckii, Clostridium butyricum, and Akkermansia muciniphila. In another non-limiting example, a composition can comprise Bifidobacterium infantis, Clostridium beijerinckii, Clostridium butyricum, and Akkermansia muciniphila. In another non-limiting example, a composition can comprise Akkermansia muciniphila and Eubacterium hallii.

A composition can comprise a therapeutically-effective amount of at least one isolated and purified microbe with a rRNA (e.g., 16S rRNA and/or 23S rRNA) sequence comprising at least about: 85%, 87%, 90%, 91%, 92%, 93%, 94%, 95%, 96%, 97%, 98%, 99%, 99.5%, or 100% sequence identity to a rRNA sequence selected from the following microbes: Clostridium beijerinckii, Clostridium butyricum, Bifidobacterium infantis, Akkermansia muciniphila, or Eubacterium hallii.

A first example of a composition (A) can comprise Clostridium beijerinckii, Clostridium butyricum, and Bifidobacterium infantis. A second example of a composition (B) can comprise Clostridium beijerinckii, Clostridium butyricum, Bifidobacterium infantis, Akkermansia muciniphila, and Eubacterium hallii. A third example of a composition (C) can comprise Clostridium butyricum, Bifidobacterium infantis, Akkermansia muciniphila, and Eubacterium hallii. A fourth example (D) can comprise Clostridium beijerinckii, Bifidobacterium infantis, Akkermansia muciniphila, and Eubacterium hallii. A fifth example of a composition (E) can comprise Clostridium beijerinckii, Akkermansia muciniphila, and Eubacterium hallii. A sixth example of a composition (F) can comprise Clostridium beijerinckii and Bifidobacteria infantis. A seventh example of a composition (G) can comprise Clostridium beijerinckii, Clostridium butyricum, Bifidobacterium infantis, Akkermansia muciniphila, and Eubacterium hallii. An eighth example of a composition (H) can comprise Clostridium beijerinckii, Clostridium butyricum, Bifidobacterium infantis, and Akkermansia muciniphila. A ninth example of a composition (I) can comprise Clostridium beijerinckii, Bifidobacteria infantis, and Akkermansia muciniphila. An tenth example of a composition (J) can comprise Clostridium butyricum, Bifidobacterium infantis, and Akkermansia muciniphila.

In other examples, the composition may include Akkermansia muciniphila along with at least one additional microbial species, which in some cases may be Eubacterium hallii, Clostridium beijerinckii, Clostridium butyricum, Bifidobacterium infantis, Bifidobacterium longum, Clostridium indolis, Acidaminococcus fermentans, Acidaminococcus intestine, Blautia hydrogenotrophica, Citrobacter amalonaticus, Eubacterium rectale, or Faecalibacterium prausnitzii. In some embodiments, a composition of the disclosure comprises Akkermansia muciniphila and Eubacterium hallii.

In other examples, the composition may include Akkermansia muciniphila along with at least two additional microbial species that may be selected independently from Eubacterium hallii, Clostridium beijerinckii, Clostridium butyricum, Bifidobacterium infantis, Bifidobacterium longum, Clostridium indolis, Acidaminococcus fermentans, Acidaminococcus intestine, Blautia hydrogenotrophica, Citrobacter amalonaticus, Eubacterium rectale, or Faecalibacterium prausnitzii. In some embodiments, a composition of the disclosure comprises Akkermansia muciniphila, Eubacterium hallii, and Bifidobacterium infantis.

In other examples, the composition may include Akkermansia muciniphila along with at least three additional microbial species that may be selected independently from Eubacterium hallii, Clostridium beijerinckii, Clostridium butyricum, Bifidobacterium infantis, Bifidobacterium longum, Clostridium indolis, Acidaminococcus fermentans, Acidaminococcus intestine, Blautia hydrogenotrophica, Citrobacter amalonaticus, Eubacterium rectale, or Faecalibacterium prausnitzii. In some embodiments, a composition of the disclosure comprises Akkermansia muciniphila, Eubacterium hallii, Biidobacterium infantis, and Clostridium beijerinckii. In some embodiments, a composition of the disclosure comprises Akkermansia muciniphila, Eubacterium hallii, Bifidobacterium infantis, and Clostridium butyricum.

In other examples, the composition may include Akkermansia muciniphila along with at least four additional microbial species that may be selected independently from Eubacterium hallii, Clostridium beijerinckii, Clostridium butyricum, Bifidobacterium infantis, Bifidobacterium longum, Clostridium indolis, Acidaminococcus fermentans, Acidaminococcus intestine, Blautia hydrogenotrophica, Citrobacter amalonaticus, Eubacterium rectale, or Faecalibacterium prausnitzii. In some embodiments, a composition of the disclosure comprises Akkermansia muciniphila, Eubacterium hallii, Biidobacterium infantis, Clostridium beijerinckii, and Clostridium butyricum.

In other examples, the composition may include Clostridium beijerinckii along with at least two additional microbial species that may be selected independently from Eubacterium hallii, Clostridium butyricum, Bifidobacterium infantis, Bifidobacterium longum, Clostridium indolis, Akkermansia muciniphila, Acidaminococcus fermentans, Acidaminococcus intestine, Blautia hydrogenotrophica, Citrobacter amalonaticus, Eubacterium rectale, or Faecalibacterium prausnitzii.

In other examples, the composition may include Clostridium beijerinckii along with at least three additional microbial species that may be selected independently from Eubacterium hallii, Clostridium butyricum, Bifidobacterium infantis, Bifidobacterium longum, Clostridium indolis, Akkermansia Muciniphila, Acidaminococcus fermentans, Acidaminococcus intestine, Blautia hydrogenotrophica, Citrobacter amalonaticus, Eubacterium rectale, or Faecalibacterium prausnitzii.

In other examples, the composition may include Clostridium beijerinckii along with at least four additional microbial species that may be selected independently from Eubacterium hallii, Clostridium butyricum, Bifidobacterium infantis, Bifidobacterium longum, Clostridium indolis, Akkermansia Muciniphila, Acidaminococcus fermentans, Acidaminococcus intestine, Blautia hydrogenotrophica, Citrobacter amalonaticus, Eubacterium rectale, or Faecalibacterium prausnitzii.

In other examples, the composition may include Blautia hydrogenotrophica along with at least two additional microbial species that may be selected independently from Eubacterium hallii, Clostridium butyricum, Bifidobacterium infantis, Bifidobacterium longum, Clostridium indolis, Akkermansia Muciniphila, Acidaminococcus fermentans, Acidaminococcus intestine, Clostridium beijerinckii, Citrobacter amalonaticus, Eubacterium rectale, or Faecalibacterium prausnitzii.

In other examples, the composition may include Blautia hydrogenotrophica along with at least three additional microbial species that may be selected independently from Eubacterium hallii, Clostridium butyricum, Bifidobacterium infantis, Bifidobacterium longum, Clostridium indolis, Akkermansia Muciniphila, Acidaminococcus fermentans, Acidaminococcus intestine, Clostridium beijerinckii, Citrobacter amalonaticus, Eubacterium rectale, or Faecalibacterium prausnitzii.

In other examples, the composition may include Blautia hydrogenotrophica along with at least four additional microbial species that may be selected independently from Eubacterium hallii, Clostridium butyricum, Bifidobacterium infantis, Bifidobacterium longum, Clostridium indolis, Akkermansia Muciniphila, Acidaminococcus fermentans, Acidaminococcus intestine, Clostridium beijerinckii, Citrobacter amalonaticus, Eubacterium rectale, or Faecalibacterium prausnitzii.

In other examples, the composition may include Acidaminococcus fermentans along with at least two additional microbial species that may be selected independently from Eubacterium hallii, Clostridium butyricum, Bifidobacterium infantis, Bifidobacterium longum, Clostridium indolis, Akkermansia Muciniphila, Blautia hydrogenotrophica, Acidaminococcus intestine, Clostridium beijerinckii, Citrobacter amalonaticus, Eubacterium rectale, or Faecalibacterium prausnitzii.

In other examples, the composition may include Acidaminococcus fermentans along with at least three additional microbial species that may be selected independently from Eubacterium hallii, Clostridium butyricum, Bifidobacterium infantis, Bifidobacterium longum, Clostridium indolis, Akkermansia Muciniphila, Blautia hydrogenotrophica, Acidaminococcus intestine, Clostridium beijerinckii, Citrobacter amalonaticus, Eubacterium rectale, or Faecalibacterium prausnitzii.

In other examples, the composition may include Acidaminococcus fermentans along with at least four additional microbial species that may be selected independently from Eubacterium hallii, Clostridium butyricum, Bifidobacterium infantis, Bifidobacterium longum, Clostridium indolis, Akkermansia Muciniphila, Blautia hydrogenotrophica, Acidaminococcus intestine, Clostridium beijerinckii, Citrobacter amalonaticus, Eubacterium rectale, or Faecalibacterium prausnitzii.

In other examples, the composition may include Clostridium butyricum along with at least two additional microbial species that may be selected independently from Eubacterium hallii, Acidaminococcus fermentans, Bifidobacterium infantis, Bifidobacterium longum, Clostridium indolis, Akkermansia Muciniphila, Blautia hydrogenotrophica, Acidaminococcus intestine, Clostridium beijerinckii, Citrobacter amalonaticus, Eubacterium rectale, or Faecalibacterium prausnitzii.

In other examples, the composition may include Clostridium butyricum along with at least three additional microbial species that may be selected independently from Eubacterium hallii, Acidaminococcus fermentans, Bifidobacterium infantis, Bifidobacterium longum, Clostridium indolis, Akkermansia Muciniphila, Blautia hydrogenotrophica, Acidaminococcus intestine, Clostridium beijerinckii, Citrobacter amalonaticus, Eubacterium rectale, or Faecalibacterium prausnitzii.

In other examples, the composition may include Clostridium butyricum along with at least four additional microbial species that may be selected independently from Eubacterium hallii, Acidaminococcus fermentans, Bifidobacterium infantis, Bifidobacterium longum, Clostridium indolis, Akkermansia Muciniphila, Blautia hydrogenotrophica, Acidaminococcus intestine, Clostridium beijerinckii, Citrobacter amalonaticus, Eubacterium rectale, or Faecalibacterium prausnitzii.

In other examples, the composition may include Bifidobacterium infantis along with at least two additional microbial species that may be selected independently from Eubacterium hallii, Acidaminococcus fermentans, Clostridium butyricum, Bifidobacterium longum, Clostridium indolis, Akkermansia Muciniphila, Blautia hydrogenotrophica, Acidaminococcus intestine, Clostridium beijerinckii, Citrobacter amalonaticus, Eubacterium rectale, or Faecalibacterium prausnitzii.

In other examples, the composition may include Bifidobacterium infantis along with at least three additional microbial species that may be selected independently from Eubacterium hallii, Acidaminococcus fermentans, Clostridium butyricum, Bifidobacterium longum, Clostridium indolis, Akkermansia Muciniphila, Blautia hydrogenotrophica, Acidaminococcus intestine, Clostridium beijerinckii, Citrobacter amalonaticus, Eubacterium rectale, or Faecalibacterium prausnitzii.

In other examples, the composition may include Bifidobacterium infantis along with at least four additional microbial species that may be selected independently from Eubacterium hallii, Acidaminococcus fermentans, Clostridium butyricum, Bifidobacterium longum, Clostridium indolis, Akkermansia Muciniphila, Blautia hydrogenotrophica, Acidaminococcus intestine, Clostridium beijerinckii, Citrobacter amalonaticus, Eubacterium rectale, or Faecalibacterium prausnitzii.

A composition can comprise one or more microbes with an rRNA sequence comprising at least about 85% sequence identity to an rRNA sequence of Eubacterium hallii. An additional composition can comprise one or more microbes with an rRNA sequence comprising at least about 85% sequence identity to an rRNA sequence of Akkermansia muciniphila. A composition can comprise one or more microbes with an rRNA sequence comprising at least about 90% sequence identity to an rRNA sequence of Clostridium beijerinckii. A composition can comprise one or more microbes with an rRNA sequence comprising at least about 90% sequence identity to an rRNA sequence of Clostridium butyricum. Some compositions can comprise one or more microbes with an rRNA sequence comprising at least about 90% sequence identity to an rRNA sequence of Bifidobacterium infantis. A composition can comprise one or more microbes with an rRNA sequence comprising at least about 90% sequence identity to an rRNA sequence of Eubacterium hallii. A composition can comprise one or more microbes with an rRNA sequence comprising at least about 90% sequence identity to an rRNA sequence of Akkermansia muciniphila. A composition can comprise one or more microbes with an rRNA sequence comprising at least about 95% sequence identity to an rRNA sequence of Clostridium beijerinckii. A composition can comprise one or more microbes with an rRNA sequence comprising at least about 95% sequence identity to an rRNA sequence of Clostridium butyricum. A composition can comprise comprises one or more microbes with an rRNA sequence comprising at least about 95% sequence identity to an rRNA sequence of Bifidobacterium infantis. A composition can comprise one or more microbes with an rRNA sequence comprising at least about 95% sequence identity to an rRNA sequence of Eubacterium hallii. A composition can comprise one or more microbes with an rRNA sequence comprising at least about 95% sequence identity to an rRNA sequence of Akkermansia muciniphila. A composition can comprise one or more microbes with an rRNA sequence comprising at least about 97% sequence identity to an rRNA sequence of Clostridium beijerinckii. A composition can comprise one or more microbes with an rRNA sequence comprising at least about 97% sequence identity to an rRNA sequence of Clostridium butyricum. A composition can comprise comprises one or more microbes with an rRNA sequence comprising at least about 97% sequence identity to an rRNA sequence of Bifidobacterium infantis. A composition can comprise one or more microbes with an rRNA sequence comprising at least about 97% sequence identity to an rRNA sequence of Eubacterium hallii. A composition can comprise one or more microbes with an rRNA sequence comprising at least about 97% sequence identity to an rRNA sequence of Akkermansia muciniphila. A composition can comprise one or more microbes with an rRNA sequence comprising at least about 98% sequence identity to an rRNA sequence of Clostridium beijerinckii. A composition can comprise one or more microbes with an rRNA sequence comprising at least about 98% sequence identity to an rRNA sequence of Clostridium butyricum. A composition can comprise comprises one or more microbes with an rRNA sequence comprising at least about 98% sequence identity to an rRNA sequence of Bifidobacterium infantis. A composition can comprise one or more microbes with an rRNA sequence comprising at least about 98% sequence identity to an rRNA sequence of Eubacterium hallii. A composition can comprise one or more microbes with an rRNA sequence comprising at least about 98% sequence identity to an rRNA sequence of Akkermansia muciniphila. A composition can comprise one or more microbes with an rRNA sequence comprising at least about 99% sequence identity to an rRNA sequence of Clostridium beijerinckii. A composition can comprise one or more microbes with an rRNA sequence comprising at least about 99% sequence identity to an rRNA sequence of Clostridium butyricum. A composition can comprise comprises one or more microbes with an rRNA sequence comprising at least about 99% sequence identity to an rRNA sequence of Bifidobacterium infantis. A composition can comprise one or more microbes with an rRNA sequence comprising at least about 99% sequence identity to an rRNA sequence of Eubacterium hallii. A composition can comprise one or more microbes with an rRNA sequence comprising at least about 99% sequence identity to an rRNA sequence of Akkermansia muciniphila. A composition can comprise one or more microbes with an rRNA sequence comprising at least about 95% sequence identity to an rRNA sequence of Clostridium beijerinckii. A composition can comprise one or more microbes with an rRNA sequence comprising at least about 95% sequence identity to an rRNA sequence of Clostridium butyricum. A composition can comprise comprises one or more microbes with an rRNA sequence comprising at least about 95% sequence identity to an rRNA sequence of Bifidobacterium infantis. A composition can comprise one or more microbes with an rRNA sequence comprising at least about 95% sequence identity to an rRNA sequence of Eubacterium hallii. A composition can comprise one or more microbes with an rRNA sequence comprising at least about 95% sequence identity to an rRNA sequence of Akkermansia muciniphila.

Microbial compositions described herein can be used in a composition comprising an effective or therapeutically-effective amount of the composition for treating a subject. A composition of the disclosure can be a combination of any microorganisms described herein with other components, such as carriers, stabilizers, diluents, dispersing agents, suspending agents, thickening agents, and excipients. The composition can facilitate administration of the microorganisms to a subject. The appropriate quantity of a therapeutic composition to be administered, the number of treatments, and unit dose can vary according to a subject and/or the disease state of the subject. A composition can be administered as a therapeutic or cosmetic.

Compositions of the disclosure can comprise isolated and purified microbes formulated in a substantially dry powder form. The isolated and purified microbes can be derived from lyophilization of microbial cultures. A lyophilized composition can be mixed with a saline or other solution prior to administration, or it may be administered as a dried form, e.g., in a capsule or tablet, or incorporated into another ingestible form, e.g., as described in greater detail below.

A composition can comprise viable microbes. For example, the microbial composition comprises microbes that can replicate once they are delivered to the target habitat (e.g. gut). In some cases, the composition may not comprise spores.

A composition of the disclosure can be in a unit dosage form. A unit dose can be a capsule, A unit dose can be a pill. A unit dose can be a food bar. A unit dose can be a powder (e.g., a powder weight or a portioned sachet of powder). A unit dose can be a liquid (e.g., a liquid in a sealed carton, bottle, can, or vial).

In some aspects, the compositions will provide viable microbes at a level of from about at least 1×10⁷ colony forming units (CFUs) per dose administered to a subject to about 1×10¹⁴ CFUs per dose, of any individual microbial species. In some cases, a composition will comprise about 1×10⁷ CFUs per dose, about 1×10⁸ CFUs per dose, about 1×10⁹ CFUs per dose, about 1×10¹⁰ CFUs per dose, about 1×10¹¹ CFUs per dose, about 1×10¹² CFUs per dose, about 1×10¹³ CFUs per dose, or about 1×10¹⁴ CFUs per dose. A dose may comprise about 1×10⁷ CFUs per gram powder, about 1×10⁸ CFUs per gram powder, about 1×10⁹ CFUs per gram powder, about 1×10¹⁰ CFUs per gram powder, about 1×10¹¹ CFUs per gram powder, about 1×10¹² CFUs per gram powder, about 1×10¹³ CFUs per gram powder, or about 1×10¹⁴ CFUs per gram powder. A dose may comprise about 1×10⁷ CFUs per 1 mL of liquid suspension, about 1×10⁸ CFUs per 1 mL of liquid suspension, about 1×10⁹ CFUs per 1 mL of liquid suspension, about 1×10¹ CFUs per 1 mL of liquid suspension, about 1×10¹¹ CFUs per 1 mL of liquid suspension, about 1×10¹² CFUs per 1 mL of liquid suspension, about 1×10¹³ CFUs per 1 mL of liquid suspension, or about 1×10¹⁴ CFUs per 1 mL of liquid suspension.

A dose or a unit dose of a composition of the disclosure can comprise, for example, at least about 1×10\hat{ }3, 1×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of a microbe disclosed herein.

In some cases, a dose or a unit dose of a composition of the disclosure comprises at most about 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of a microbe disclosed herein.

In some embodiments, a dose or a unit dose of a composition of the disclosure can comprise, for example, about 1×10\hat{ }3, 1×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of a microbe disclosed herein.

A dose or a unit dose of a composition of the disclosure can comprise, for example, at least about 1×10\hat{ }3, 1×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of Akkermansia muciniphila, a microbe comprising an rRNA sequence (e.g., 16S rRNA or 23S rRNA) with at least about 97% sequence identity to an rRNA from Akkermansia muciniphila, or a microbe comprising an rRNA sequence with at least about 97% sequence identity to any one of SEQ ID NOS: 1-6.

In some cases, a dose or a unit dose of a composition of the disclosure comprises at most about 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of Akkermansia muciniphila, a microbe comprising an rRNA sequence (e.g., 16S rRNA or 23S rRNA) with at least about 97% sequence identity to an rRNA from Akkermansia muciniphila, or a microbe comprising an rRNA sequence with at least about 97% sequence identity to any one of SEQ ID NOS: 1-6.

In some embodiments, a dose or a unit dose of a composition of the disclosure can comprise, for example, about 1×10\hat{ }3, 1×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of Akkermansia muciniphila, a microbe comprising an rRNA sequence (e.g., 16S rRNA or 23S rRNA) with at least about 97% sequence identity to an rRNA from Akkermansia muciniphila, or a microbe comprising an rRNA sequence with at least about 97% sequence identity to any one of SEQ ID NOS: 1-6. In some embodiments, a dose or a unit dose of a composition of the disclosure can comprise, for example, about 1.9×10\hat{ }8 CFUs of Akkermansia muciniphila, a microbe comprising an rRNA sequence (e.g., 16S rRNA or 23S rRNA) with at least about 97% sequence identity to an rRNA from Akkermansia muciniphila, or a microbe comprising an rRNA sequence with at least about 97% sequence identity to any one of SEQ ID NOS: 1-6. In some embodiments, a dose or a unit dose of a composition of the disclosure can comprise, for example, about 3.9×10\hat{ }8 CFUs of Akkermansia muciniphila, a microbe comprising an rRNA sequence (e.g., 16S rRNA or 23S rRNA) with at least about 97% sequence identity to an rRNA from Akkermansia muciniphila, or a microbe comprising an rRNA sequence with at least about 97% sequence identity to any one of SEQ ID NOS: 1-6. In some embodiments, a dose or a unit dose of a composition of the disclosure can comprise, for example, about 1.2×10\hat{ }9 CFUs of Akkermansia muciniphila, a microbe comprising an rRNA sequence (e.g., 16S rRNA or 23S rRNA) with at least about 97% sequence identity to an rRNA from Akkermansia muciniphila, or a microbe comprising an rRNA sequence with at least about 97% sequence identity to any one of SEQ ID NOS: 1-6.

A dose or a unit dose of a composition of the disclosure can comprise, for example, at least about 1×10\hat{ }3, 1×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of Eubacterium hallii or a microbe comprising an rRNA sequence (e.g., 16S rRNA or 23S rRNA) with at least about 97% sequence identity to an rRNA from Eubacterium hallii.

In some cases, a dose or a unit dose of a composition of the disclosure comprises at most about 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of Eubacterium hallii or a microbe comprising an rRNA sequence (e.g., 16S rRNA or 23S rRNA) with at least about 97% sequence identity to an rRNA from Eubacterium hallii.

In some embodiments, a dose or a unit dose of a composition of the disclosure can comprise, for example, about 1×10\hat{ }3, 1×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of Eubacterium hallii or a microbe comprising an rRNA sequence (e.g., 16S rRNA or 23S rRNA) with at least about 97% sequence identity to an rRNA from Eubacterium hallii. In some embodiments, a dose or a unit dose of a composition of the disclosure can comprise, for example, about 1.5×10\hat{ }8 CFUs of Eubacterium hallii or a microbe comprising an rRNA sequence (e.g., 16S rRNA or 23S rRNA) with at least about 97% sequence identity to an rRNA from Eubacterium hallii. In some embodiments, a dose or a unit dose of a composition of the disclosure can comprise, for example, about 3×10\hat{ }8 CFUs of Eubacterium hallii or a microbe comprising an rRNA sequence (e.g., 16S rRNA or 23S rRNA) with at least about 97% sequence identity to an rRNA from Eubacterium hallii. In some embodiments, a dose or a unit dose of a composition of the disclosure can comprise, for example, about 9×10\hat{ }9 CFUs of Eubacterium hallii or a microbe comprising an rRNA sequence (e.g., 16S rRNA or 23S rRNA) with at least about 97% sequence identity to an rRNA from Eubacterium hallii.

A dose or a unit dose of a composition of the disclosure can comprise, for example, at least about 1×10\hat{ }3, 1×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of Bifidobacterium infantis or a microbe comprising an rRNA sequence (e.g., 16S rRNA or 23S rRNA) with at least about 97% sequence identity to an rRNA from Bifidobacterium infantis.

In some cases, a dose or a unit dose of a composition of the disclosure comprises at most about 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of Bifidobacterium infantis or a microbe comprising an rRNA sequence (e.g., 16S rRNA or 23S rRNA) with at least about 97% sequence identity to an rRNA from Bifidobacterium infantis.

In some embodiments, a dose or a unit dose of a composition of the disclosure can comprise, for example, about 1×10\hat{ }3, 1×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of Bifidobacterium infantis or a microbe comprising an rRNA sequence (e.g., 16S rRNA or 23S rRNA) with at least about 97% sequence identity to an rRNA from Bifidobacterium infantis. In some embodiments, a dose or a unit dose of a composition of the disclosure can comprise, for example, about 3.3×10\hat{ }7 CFUs of Bifidobacterium infantis or a microbe comprising an rRNA sequence (e.g., 16S rRNA or 23S rRNA) with at least about 97% sequence identity to an rRNA from Bifidobacterium infantis. In some embodiments, a dose or a unit dose of a composition of the disclosure can comprise, for example, about 6.7×10\hat{ }7 CFUs of Bifidobacterium infantis or a microbe comprising an rRNA sequence (e.g., 16S rRNA or 23S rRNA) with at least about 97% sequence identity to an rRNA from Bifidobacterium infantis. In some embodiments, a dose or a unit dose of a composition of the disclosure can comprise, for example, about 2×10\hat{ }8 CFUs of Bifidobacterium infantis or a microbe comprising an rRNA sequence (e.g., 16S rRNA or 23S rRNA) with at least about 97% sequence identity to an rRNA from Bifidobacterium infantis.

A dose or a unit dose of a composition of the disclosure can comprise, for example, at least about 1×10\hat{ }3, 1×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of Clostridium beijerinckii or a microbe comprising an rRNA sequence (e.g., 16S rRNA or 23S rRNA) with at least about 97% sequence identity to an rRNA from Clostridium beijerinckii.

In some cases, a dose or a unit dose of a composition of the disclosure comprises at most about 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of Clostridium beijerinckii or a microbe comprising an rRNA sequence (e.g., 16S rRNA or 23S rRNA) with at least about 97% sequence identity to an rRNA from Clostridium beijerinckii.

In some embodiments, a dose or a unit dose of a composition of the disclosure can comprise, for example, about 1×10\hat{ }3, 1×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of Clostridium beijerinckii or a microbe comprising an rRNA sequence (e.g., 16S rRNA or 23S rRNA) with at least about 97% sequence identity to an rRNA from Clostridium beijerinckii. In some embodiments, a dose or a unit dose of a composition of the disclosure can comprise, for example, about 1.9×10\hat{ }8 CFUs of Clostridium beijerinckii or a microbe comprising an rRNA sequence (e.g., 16S rRNA or 23S rRNA) with at least about 97% sequence identity to an rRNA from Clostridium beijerinckii. In some embodiments, a dose or a unit dose of a composition of the disclosure can comprise, for example, about 3.8×10\hat{ }8 CFUs of Clostridium beijerinckii or a microbe comprising an rRNA sequence (e.g., 16S rRNA or 23S rRNA) with at least about 97% sequence identity to an rRNA from Clostridium beijerinckii. In some embodiments, a dose or a unit dose of a composition of the disclosure can comprise, for example, about 1.2×10\hat{ }9 CFUs of Clostridium beijerinckii or a microbe comprising an rRNA sequence (e.g., 16S rRNA or 23S rRNA) with at least about 97% sequence identity to an rRNA from Clostridium beijerinckii.

A dose or a unit dose of a composition of the disclosure can comprise, for example, at least about 1×10\hat{ }3, 1×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of Clostridium butyricum or a microbe comprising an rRNA sequence (e.g., 16S rRNA or 23S rRNA) with at least about 97% sequence identity to an rRNA from Clostridium butyricum.

In some cases, a dose or a unit dose of a composition of the disclosure comprises at most about 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of Clostridium butyricum or a microbe comprising an rRNA sequence (e.g., 16S rRNA or 23S rRNA) with at least about 97% sequence identity to an rRNA from Clostridium butyricum.

In some embodiments, a dose or a unit dose of a composition of the disclosure can comprise, for example, about 1×10\hat{ }3, 1×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of Clostridium butyricum or a microbe comprising an rRNA sequence (e.g., 16S rRNA or 23S rRNA) with at least about 97% sequence identity to an rRNA from Clostridium butyricum. In some embodiments, a dose or a unit dose of a composition of the disclosure can comprise, for example, about 5.6×10\hat{ }7 CFUs of Clostridium butyricum or a microbe comprising an rRNA sequence (e.g., 16S rRNA or 23S rRNA) with at least about 97% sequence identity to an rRNA from Clostridium butyricum. In some embodiments, a dose or a unit dose of a composition of the disclosure can comprise, for example, about 1.1×10\hat{ }8 CFUs of Clostridium butyricum or a microbe comprising an rRNA sequence (e.g., 16S rRNA or 23S rRNA) with at least about 97% sequence identity to an rRNA from Clostridium butyricum. In some embodiments, a dose or a unit dose of a composition of the disclosure can comprise, for example, about 3.3×10\hat{ }8 CFUs of Clostridium butyricum or a microbe comprising an rRNA sequence (e.g., 16S rRNA or 23S rRNA) with at least about 97% sequence identity to an rRNA from Clostridium butyricum.

A dose or a unit dose of a composition of the disclosure can comprise, for example, at least about 1×10\hat{ }3, 1×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of total combined microbes.

In some cases, a dose or a unit dose of a composition of the disclosure comprises at most about 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of total combined microbes.

In some embodiments, a dose or a unit dose of a composition of the disclosure can comprise, for example, about 1×10\hat{ }3, 1×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of total combined microbes. In some embodiments, a dose or a unit dose of a composition of the disclosure can comprise, for example, about 6.2×10\hat{ }8 CFUs of total combined microbes. In some embodiments, a dose or a unit dose of a composition of the disclosure can comprise, for example, about 1.3×10\hat{ }9 CFUs of total combined microbes. In some embodiments, a dose or a unit dose of a composition of the disclosure can comprise, for example, about 3.7×10\hat{ }9 CFUs of total combined microbes.

A dose or a unit dose of a composition of the disclosure can comprise, for example, at least about 1×10\hat{ }3, 1×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of a mucin-regulating and/or primary fermenter microbe.

In some cases, a dose or a unit dose of a composition of the disclosure comprises at most about 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of a mucin-regulating and/or primary fermenter microbe.

In some embodiments, a dose or a unit dose of a composition of the disclosure can comprise, for example, about 1×10\hat{ }3, 1×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of a mucin-regulating and/or primary fermenter microbe.

A dose or a unit dose of a composition of the disclosure can comprise, for example, at least about 1×10\hat{ }3, 1×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of a butyrate-producing and/or secondary fermenter microbe.

In some cases, a dose or a unit dose of a composition of the disclosure comprises at most about 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of a butyrate-producing and/or secondary fermenter microbe.

In some embodiments, a dose or a unit dose of a composition of the disclosure can comprise, for example, about 1×10\hat{ }3, 1×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, or 1×10\hat{ }17 CFUs of a butyrate-producing and/or secondary fermenter microbe.

In some cases, where a composition of the disclosure comprises two or more different microbes, one of the microbes can comprise at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose.

In some instances, where a composition of the disclosure comprises two or more different microbes, one of the microbes can comprise at most 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35% 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose.

In some embodiments, where a composition of the disclosure comprises two or more different microbes, one of the microbes can comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose.

In some embodiments, where a composition of the disclosure comprises two or more different microbes, one of the microbes can comprise about 0% to about 5%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, about 95% to about 99%, about 0% to about 10%, About 1% to about 10%, about 10% to about 20%, about 20% to about 30%, about 30% to about 40%, about 40% to about 50%, about 50% to about 60%, about 60% to about 70%, about 70% to about 80%, about 80% to about 90% about 90% to about 99%, about 0% to about 20%, about 20% to about 40%, about 40% to about 60%, about 60% to about 80%, about 80% to about 99%, about 0% to about 40% about 1% to about 40%, about 40% to about 80%, about 10% to about 50%, about 50% to about 90%, about 30% to about 70%, about 50% to about 90%, about 0% to about 30%, about 1% to about 30%, about 10% to about 40%, about 20% to about 50%, about 30% to about 80%, about 40% to about 70%, about 50% to about 80%, or about 60% to about 90% of the total CFUs in the composition or unit dose.

In some cases, where a composition of the disclosure comprises two or more different microbes, at least 1%, 2%, 3%, 4%, 50%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose can be Akkermansia muciniphila, a microbe comprising an rRNA sequence (e.g., 16S rRNA or 23S rRNA) with at least about 97% sequence identity to an rRNA from Akkermansia muciniphila, or a microbe comprising an rRNA sequence with at least about 97% sequence identity to any one of SEQ ID NOS: 1-6.

In some instances, where a composition of the disclosure comprises two or more different microbes, at most 1%, 2%, 3%, 4%, 50%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose can be Akkermansia muciniphila, a microbe comprising an rRNA sequence (e.g., 16S rRNA or 23S rRNA) with at least about 97% sequence identity to an rRNA from Akkermansia muciniphila, or a microbe comprising an rRNA sequence with at least about 97% sequence identity to any one of SEQ ID NOS: 1-6.

In some embodiments, where a composition of the disclosure comprises two or more different microbes, about 1%, 2%, 3%, 4%, 5%, 6%, 7%, 8%, 9%, 10%, 11%, 12%, 13%, 14%, 15% 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose can be Akkermansia muciniphila, a microbe comprising an rRNA sequence (e.g., 16S rRNA or 23S rRNA) with at least about 97% sequence identity to an rRNA from Akkermansia muciniphila, or a microbe comprising an rRNA sequence with at least about 97% sequence identity to any one of SEQ ID NOS: 1-6.

In some embodiments, where a composition of the disclosure comprises two or more different microbes, about 0% to about 5%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, about 95% to about 99%, about 0% to about 10%, About 1% to about 10%, about 10% to about 20%, about 20% to about 30%, about 30% to about 40%, about 40% to about 50%, about 50% to about 60%, about 60% to about 70%, about 70% to about 80%, about 80% to about 90% about 90% to about 99%, about 0% to about 20%, about 20% to about 40%, about 40% to about 60%, about 60% to about 80%, about 80% to about 99%, about 0% to about 40% about 1% to about 40%, about 40% to about 80%, about 10% to about 50%, about 50% to about 90%, about 30% to about 70%, about 50% to about 90%, about 0% to about 30%, about 1% to about 30%, about 10% to about 40%, about 20% to about 50%, about 30% to about 80%, about 40% to about 70%, about 50% to about 80%, or about 60% to about 90% of the total CFUs in the composition or unit dose can be Akkermansia muciniphila, a microbe comprising an rRNA sequence (e.g., 16S rRNA or 23S rRNA) with at least about 97% sequence identity to an rRNA from Akkermansia muciniphila, or a microbe comprising an rRNA sequence with at least about 97% sequence identity to any one of SEQ ID NOS: 1-6.

In some cases, where a composition of the disclosure comprises two or more different microbes, at least 1%, 2%, 3%, 4%, 50%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose can be Eubacterium hallii or a microbe comprising an rRNA sequence (e.g., 16S rRNA or 23S rRNA) with at least about 97% sequence identity to an rRNA from Eubacterium hallii.

In some instances, where a composition of the disclosure comprises two or more different microbes, at most 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose can be Eubacterium hallii or a microbe comprising an rRNA sequence (e.g., 16S rRNA or 23S rRNA) with at least about 97% sequence identity to an rRNA from Eubacterium hallii.

In some embodiments, where a composition of the disclosure comprises two or more different microbes, about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose can be Eubacterium hallii or a microbe comprising an rRNA sequence (e.g., 16S rRNA or 23S rRNA) with at least about 97% sequence identity to an rRNA from Eubacterium hallii.

In some embodiments, where a composition of the disclosure comprises two or more different microbes, about 0% to about 5%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, about 95% to about 99%, about 0% to about 10%, About 1% to about 10%, about 10% to about 20%, about 20% to about 30%, about 30% to about 40%, about 40% to about 50%, about 50% to about 60%, about 60% to about 70%, about 70% to about 80%, about 80% to about 90% about 90% to about 99%, about 0% to about 20%, about 20% to about 40%, about 40% to about 60%, about 60% to about 80%, about 80% to about 99%, about 0% to about 40% about 1% to about 40%, about 40% to about 80%, about 10% to about 50%, about 50% to about 90%, about 30% to about 70%, about 50% to about 90%, about 0% to about 30%, about 1% to about 30%, about 10% to about 40%, about 20% to about 50%, about 30% to about 80%, about 40% to about 70%, about 50% to about 80%, or about 60% to about 90% of the total CFUs in the composition or unit dose can be Eubacterium hallii or a microbe comprising an rRNA sequence (e.g., 16S rRNA or 23S rRNA) with at least about 97% sequence identity to an rRNA from Eubacterium hallii.

In some cases, where a composition of the disclosure comprises two or more different microbes, at least 1%, 2%, 33%, 4%, 50%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose can be Bifidobacterium infantis or a microbe comprising an rRNA sequence (e.g., 16S rRNA or 23S rRNA) with at least about 97% sequence identity to an rRNA from Bifidobacterium infantis.

In some instances, where a composition of the disclosure comprises two or more different microbes, at most 1%, 2%, 33%, 4%, 50%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose can be Bifidobacterium infantis or a microbe comprising an rRNA sequence (e.g., 16S rRNA or 23S rRNA) with at least about 97% sequence identity to an rRNA from Bifidobacterium infantis.

In some embodiments, where a composition of the disclosure comprises two or more different microbes, about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose can be Bifidobacterium infantis or a microbe comprising an rRNA sequence (e.g., 16S rRNA or 23S rRNA) with at least about 97% sequence identity to an rRNA from Bifidobacterium infantis.

In some embodiments, where a composition of the disclosure comprises two or more different microbes, about 0% to about 5%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, about 95% to about 99%, about 0% to about 10%, About 1% to about 10%, about 10% to about 20%, about 20% to about 30%, about 30% to about 40%, about 40% to about 50%, about 50% to about 60%, about 60% to about 70%, about 70% to about 80%, about 80% to about 90% about 90% to about 99%, about 0% to about 20%, about 20% to about 40%, about 40% to about 60%, about 60% to about 80%, about 80% to about 99%, about 0% to about 40% about 1% to about 40%, about 40% to about 80%, about 10% to about 50%, about 50% to about 90%, about 30% to about 70%, about 50% to about 90%, about 0% to about 30%, about 1% to about 30%, about 10% to about 40%, about 20% to about 50%, about 30% to about 80%, about 40% to about 70%, about 50% to about 80%, or about 60% to about 90% of the total CFUs in the composition or unit dose can be Bifidobacterium infantis or a microbe comprising an rRNA sequence (e.g., 16S rRNA or 23S rRNA) with at least about 97% sequence identity to an rRNA from Bifidobacterium infantis.

In some cases, where a composition of the disclosure comprises two or more different microbes, at least 1%, 2%, 3%, 4%, 50%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose can be Clostridium beijerinckii or a microbe comprising an rRNA sequence (e.g., 16S rRNA or 23S rRNA) with at least about 97% sequence identity to an rRNA from Clostridium beijerinckii.

In some instances, where a composition of the disclosure comprises two or more different microbes, at most 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose can be Clostridium beijerinckii or a microbe comprising an rRNA sequence (e.g., 16S rRNA or 23S rRNA) with at least about 97% sequence identity to an rRNA from Clostridium beijerinckii.

In some embodiments, where a composition of the disclosure comprises two or more different microbes, about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose can be Clostridium beijerinckii or a microbe comprising an rRNA sequence (e.g., 16S rRNA or 23S rRNA) with at least about 97% sequence identity to an rRNA from Clostridium beijerinckii.

In some embodiments, where a composition of the disclosure comprises two or more different microbes, about 0% to about 5%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, about 95% to about 99%, about 0% to about 10%, About 1% to about 10%, about 10% to about 20%, about 20% to about 30%, about 30% to about 40%, about 40% to about 50%, about 50% to about 60%, about 60% to about 70%, about 70% to about 80%, about 80% to about 90% about 90% to about 99%, about 0% to about 20%, about 20% to about 40%, about 40% to about 60%, about 60% to about 80%, about 80% to about 99%, about 0% to about 40% about 1% to about 40%, about 40% to about 80%, about 10% to about 50%, about 50% to about 90%, about 30% to about 70%, about 50% to about 90%, about 0% to about 30%, about 1% to about 30%, about 10% to about 40%, about 20% to about 50%, about 30% to about 80%, about 40% to about 70%, about 50% to about 80%, or about 60% to about 90% of the total CFUs in the composition or unit dose can be Clostridium beijerinckii or a microbe comprising an rRNA sequence (e.g., 16S rRNA or 23S rRNA) with at least about 97% sequence identity to an rRNA from Clostridium beijerinckii.

In some cases, where a composition of the disclosure comprises two or more different microbes, at least 1%, 2%, 3%, 4%, 50%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose can be Clostridium butyricum or a microbe comprising an rRNA sequence (e.g., 16S rRNA or 23S rRNA) with at least about 97% sequence identity to an rRNA from Clostridium butyricum.

In some instances, where a composition of the disclosure comprises two or more different microbes, at most 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose can be Clostridium butyricum or a microbe comprising an rRNA sequence (e.g., 16S rRNA or 23S rRNA) with at least about 97% sequence identity to an rRNA from Clostridium butyricum.

In some embodiments, where a composition of the disclosure comprises two or more different microbes, about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose can be Clostridium butyricum or a microbe comprising an rRNA sequence (e.g., 16S rRNA or 23S rRNA) with at least about 97% sequence identity to an rRNA from Clostridium butyricum.

In some embodiments, where a composition of the disclosure comprises two or more different microbes, about 0% to about 5%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, about 95% to about 99%, about 0% to about 10%, About 1% to about 10%, about 10% to about 20%, about 20% to about 30%, about 30% to about 40%, about 40% to about 50%, about 50% to about 60%, about 60% to about 70%, about 70% to about 80%, about 80% to about 90% about 90% to about 99%, about 0% to about 20%, about 20% to about 40%, about 40% to about 60%, about 60% to about 80%, about 80% to about 99%, about 0% to about 40% about 1% to about 40%, about 40% to about 80%, about 10% to about 50%, about 50% to about 90%, about 30% to about 70%, about 50% to about 90%, about 0% to about 30%, about 1% to about 30%, about 10% to about 40%, about 20% to about 50%, about 30% to about 80%, about 40% to about 70%, about 50% to about 80%, or about 60% to about 90% of the total CFUs in the composition or unit dose can be Clostridium butyricum or a microbe comprising an rRNA sequence (e.g., 16S rRNA or 23S rRNA) with at least about 97% sequence identity to an rRNA from Clostridium butyricum.

In some cases, where a composition of the disclosure comprises at least one mucin-regulating microbe and/or at least one primary fermenter, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose can be the at least one mucin-regulating microbe and/or the at least one primary fermenter.

In some instances, where a composition of the disclosure comprises at least one mucin-regulating microbe and/or at least one primary fermenter, at most 1%, 2%, 3%, 4%, 5%, 6%, 7%, 8%, 9% 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose can be the at least one mucin-regulating microbe and/or the at least one primary fermenter.

In some embodiments, where a composition of the disclosure comprises at least one mucin-regulating microbe and/or at least one primary fermenter, about 1%, 2%, 3%, 4%, 5%, 6%, 7%, 8%, 9% 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose can be the at least one mucin-regulating microbe and/or the at least one primary fermenter.

In some embodiments, where a composition of the disclosure comprises at least one mucin-regulating microbe and/or at least one primary fermenter, about 0% to about 5%, about 1% to about 5%, about 5% to about 10%, about to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, about 95% to about 99%, about 0% to about 10%, About 1% to about 10%, about 10% to about 20%, about 20% to about 30%, about 30% to about 40%, about 40% to about 50%, about 50% to about 60%, about 60% to about 70%, about 70% to about 80%, about 80% to about 90% about 90% to about 99%, about 0% to about 20%, about 20% to about 40%, about 40% to about 60%, about 60% to about 80%, about 80% to about 99%, about 0% to about 40% about 1% to about 40%, about 40% to about 80%, about 10% to about 50%, about 50% to about 90%, about 30% to about 70%, about 50% to about 90%, about 0% to about 30%, about 1% to about 30%, about 10% to about 40%, about 20% to about 50%, about 30% to about 80%, about 40% to about 70%, about 50% to about 80%, or about 60% to about 90% of the total CFUs in the composition or unit dose can be the at least one mucin-regulating microbe and/or the at least one primary fermenter.

In some cases, where a composition of the disclosure comprises at least one butyrate-producing microbe and/or at least one secondary fermenter, at least 1%, 2%, 3%, 4%, 5%, 6%, 7%, 8%, 9% 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose can be the at least one butyrate-producing microbe and/or the at least one secondary fermenter.

In some instances, where a composition of the disclosure comprises at least one butyrate-producing microbe and/or at least one secondary fermenter, at most 1%, 2%, 3%, 4%, 5%, 6%, 7%, 8%, 9% 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose can be the at least one butyrate-producing microbe and/or the at least one secondary fermenter.

In some embodiments, where a composition of the disclosure comprises at least one butyrate-producing microbe and/or at least one secondary fermenter, about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, of 95% of the total CFUs in the composition or unit dose can be the at least one butyrate-producing microbe and/or the at least one secondary fermenter.

In some embodiments, where a composition of the disclosure comprises at least one butyrate-producing microbe and/or at least one secondary fermenter, about 0% to about 5%, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, about 95% to about 99%, about 0% to about 10%, About 1% to about 10%, about 10% to about 20%, about 20% to about 30%, about 30% to about 40%, about 40% to about 50%, about 50% to about 60%, about 60% to about 70%, about 70% to about 80%, about 80% to about 90% about 90% to about 99%, about 0% to about 20%, about 20% to about 40%, about 40% to about 60%, about 60% to about 80%, about 80% to about 99%, about 0% to about 40% about 1% to about 40%, about 40% to about 80%, about 10% to about 50%, about 50% to about 90%, about 30% to about 70%, about 50% to about 90%, about 0% to about 30%, about 1% to about 30%, about 10% to about 40%, about 20% to about 50%, about 30% to about 80%, about 40% to about 70%, about 50% to about 80%, or about 60% to about 90% of the total CFUs in the composition or unit dose can be the at least one butyrate-producing microbe and/or the at least one secondary fermenter.

A unit dose can comprise one or more prebiotics of the disclosure (for example, inulin, chicory inulin, chicory inulin and oligofructose, complex carbohydrates, complex sugars, resistant dextrins, resistant starch, amino acids, peptides, nutritional compounds, biotin, polydextrose, oligosaccharides, polysaccharide, fructooligosaccharide (FOS), fructans, soluble fiber, insoluble fiber, fiber, starch, galactooligosaccharides (GOS), lignin, psyllium, chitin, chitosan, gums (e.g. guar gum), high amylose cornstarch (HAS), cellulose, β-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), locust bean gum, P-glucan, or methylcellulose).

A dose or a unit dose can comprise at least about 0.001 mg, 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1 mg, 1.5 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 41 mg, 42 mg, 43 mg, 44 mg, 45 mg, 46 mg, 47 mg, 48 mg, 49 mg, 50 mg, 60 mg, 70 mg, 80 mg, 85 mg, 90 mg, 91 mg, 92 mg, 93 mg, 94 mg, 95 mg, 96 mg, 97 mg, 98 mg, 99 mg, 100 mg, 150 mg, 200 mg, 250 mg, 260 mg, 265 mg, 270 mg, 275 mg, 280 mg, 285 mg, 290 mg, 295 mg, 300 mg, 400 mg, 500 mg, 600 mg, 700 mg, 800 mg, 900 mg, 1 g, 1.5 g, 2 g, 3 g, 4 g, 5 g, 6 g, 7 g, 8 g, 9 g, 10 g, 11 g, 12 g, 13 g, 14 g, 15 g, 16 g, 17 g, 18 g, 19 g, 20 g, 25 g, 30 g, 35 g, 40 g, 45 g, 50 g, 60 g, 70 g, 80 g, 90 g, 100 g of a prebiotic of the disclosure (for example, inulin).

In some cases, a dose or a unit dose can comprise at most about 0.001 mg, 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1 mg, 1.5 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 41 mg, 42 mg, 43 mg, 44 mg, 45 mg, 46 mg, 47 mg, 48 mg, 49 mg, 50 mg, 60 mg, 70 mg, 80 mg, 85 mg, 90 mg, 91 mg, 92 mg, 93 mg, 94 mg, 95 mg, 96 mg, 97 mg, 98 mg, 99 mg, 100 mg, 150 mg, 200 mg, 250 mg, 260 mg, 265 mg, 270 mg, 275 mg, 280 mg, 285 mg, 290 mg, 295 mg, 300 mg, 310 mg, 320 mg, 330 mg, 340 mg, 350 mg, 360 mg, 370 mg, 380 mg, 390 mg, 400 mg, 450 mg, 500 mg, 600 mg, 700 mg, 800 mg, 900 mg, 1 g, 1.5 g, 2 g, 3 g, 4 g, 5 g, 6 g, 7 g, 8 g, 9 g, 10 g, 11 g, 12 g, 13 g, 14 g, 15 g, 16 g, 17 g, 18 g, 19 g, 20 g, 25 g, 30 g, 35 g, 40 g, 45 g, 50 g, 60 g, 70 g, 80 g, 90 g, 100 g of a prebiotic of the disclosure (for example, inulin).

In some embodiments, a dose or a unit dose can comprise about 0.001 mg, 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1 mg, 1.5 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 41 mg, 42 mg, 43 mg, 44 mg, 45 mg, 46 mg, 47 mg, 48 mg, 49 mg, 50 mg, 60 mg, 70 mg, 80 mg, 85 mg, 90 mg, 91 mg, 92 mg, 93 mg, 94 mg, 95 mg, 96 mg, 97 mg, 98 mg, 99 mg, 100 mg, 150 mg, 200 mg, 250 mg, 260 mg, 265 mg, 270 mg, 275 mg, 276 mg, 280 mg, 285 mg, 290 mg, 295 mg, 300 mg, 400 mg, 500 mg, 600 mg, 700 mg, 800 mg, 900 mg, 1 g, 1.5 g, 2 g, 3 g, 4 g, 5 g, 6 g, 7 g, 8 g, 9 g, 10 g, 11 g, 12 g, 13 g, 14 g, 15 g, 16 g, 17 g, 18 g, 19 g, 20 g, 25 g, 30 g, 35 g, 40 g, 45 g, 50 g, 60 g, 70 g, 80 g, 90 g, 100 g of a prebiotic of the disclosure (for example, inulin). In some embodiments, a dose or a unit dose can comprise about 46 mg of inulin. In some embodiments, a dose or a unit dose can comprise about 92 mg of inulin. In some embodiments, a dose or a unit dose can comprise about 276 mg of inulin.

In some cases, a dose or a unit dose can comprise between about 0.01 mg to about 100 g, 0.01 mg to about 10 g, 0.01 mg to about 1 g, 0.01 mg to about 500 mg, 0.01 mg to about 300 mg, 0.01 mg to about 200 mg, 0.01 mg to about 100 mg, 0.01 mg to about 90 mg, 0.01 mg to about 80 mg, 0.01 mg to about 70 mg, 0.01 mg to about 60 mg, 0.01 mg to about 50 mg, 0.01 mg to about 40 mg, 0.01 mg to about 30 mg, 0.01 mg to about 20 mg, 0.01 mg to about 10 mg, 1 mg to about 500 mg, 1 mg to about 300 mg, 1 mg to about 200 mg, 1 mg to about 100 mg, 1 mg to about 90 mg, 1 mg to about 80 mg, 1 mg to about 70 mg, 1 mg to about 60 mg, 1 mg to about 50 mg, 1 mg to about 40 mg, 1 mg to about 30 mg, 1 mg to about 20 mg, 1 mg to about 10 mg, about 100 mg to about 800 mg, about 50 mg to about 100 mg, about 100 mg to about 200 mg, about 200 mg to about 300 mg, about 300 mg to about 400 mg, about 400 mg to about 500 mg, about 500 mg to about 600 mg, about 600 mg to about 700 mg, about 700 mg to about 800 mg, about 800 mg to about 900 mg, about 900 mg to about 1 g, about 1 g to about 2 g, about 2 g to about 10 g, about 10 g to about 100 g, about 10 mg to about 100 mg, about 50 mg to about 50 g, about 100 mg to about 30 g, about 200 mg to about 20 g, about 300 mg to about 15 g, about 500 mg to about 10 g, about 1 g to about 25 g, about 1 g to about 20 g, about 1 g to about 15 g, about 10 mg to about 100 mg, about 20 mg to about 90 mg, about 30 mg to about 60 mg, about 40 mg to about 50 mg, about 45 mg to about 47 mg, about 60 mg to about 120 mg, about 70 mg to about 110 mg, about 80 mg to about 100 mg, about 85 mg to about 95 mg, about 90 mg to about 100 mg, about 91 mg to about 93 mg, about 250 mg to about 300 mg, about 260 mg to about 290 mg, about 270 mg to about 280 mg, or about 275 mg to about 277 mg of a prebiotic of the disclosure.

In some cases, a dose or a unit dose can comprise between about 0.01 mg to about 100 g, 0.01 mg to about 10 g, 0.01 mg to about 1 g, 0.01 mg to about 500 mg, 0.01 mg to about 300 mg, 0.01 mg to about 200 mg, 0.01 mg to about 100 mg, 0.01 mg to about 90 mg, 0.01 mg to about 80 mg, 0.01 mg to about 70 mg, 0.01 mg to about 60 mg, 0.01 mg to about 50 mg, 0.01 mg to about 40 mg, 0.01 mg to about 30 mg, 0.01 mg to about 20 mg, 0.01 mg to about 10 mg, 1 mg to about 500 mg, 1 mg to about 300 mg, 1 mg to about 200 mg, 1 mg to about 100 mg, 1 mg to about 90 mg, 1 mg to about 80 mg, 1 mg to about 70 mg, 1 mg to about 60 mg, 1 mg to about 50 mg, 1 mg to about 40 mg, 1 mg to about 30 mg, 1 mg to about 20 mg, 1 mg to about 10 mg, about 100 mg to about 800 mg, about 50 mg to about 100 mg, about 100 mg to about 200 mg, about 200 mg to about 300 mg, about 300 mg to about 400 mg, about 400 mg to about 500 mg, about 500 mg to about 600 mg, about 600 mg to about 700 mg, about 700 mg to about 800 mg, about 800 mg to about 900 mg, about 900 mg to about 1 g, about 1 g to about 2 g, about 2 g to about 10 g, about 10 g to about 100 g, about 10 mg to about 100 mg, about 50 mg to about 50 g, about 100 mg to about 30 g, about 200 mg to about 20 g, about 300 mg to about 15 g, about 500 mg to about 10 g, about 1 g to about 25 g, about 1 g to about 20 g, about 1 g to about 15 g, about 10 mg to about 100 mg, about 20 mg to about 90 mg, about 30 mg to about 60 mg, about 40 mg to about 50 mg, about 45 mg to about 47 mg, about 60 mg to about 120 mg, about 70 mg to about 110 mg, about 80 mg to about 100 mg, about 85 mg to about 95 mg, about 90 mg to about 100 mg, about 91 mg to about 93 mg, about 250 mg to about 300 mg, about 260 mg to about 290 mg, about 270 mg to about 280 mg, or about 275 mg to about 277 mg of inulin.

A dose administered to a subject may comprise one or more unit doses (e.g., tablets or capsules) administered at a time. In some cases, a dose may include 1, 2, 3, 4, 5, 6, 7, 8, 9, 10 or more capsules or tablets administered at substantially the same time. In certain cases, a dose will comprise from about 1 to about 6 capsules or tablets administered at substantially the same time. In some cases, a dose may comprise about 1×10⁷ CFUs per tablet or capsule, about 1×10⁸ CFUs per tablet or capsule, about 1×10⁹ CFUs per tablet or capsule, about 1×10¹⁰ CFUs per tablet or capsule, about 1×10¹¹ CFUs per tablet or capsule, about 1×10¹² CFUs per tablet or capsule, about 1×10¹³ CFUs per tablet or capsule, or about 1×10¹⁴ CFUs per tablet or capsule.

A dose may be administered 5 times per day, 4 times per day, 3 times per day, 2 times per day, or 1 time per day. A dose may be administered every other day. A dose may be administered 1 time per week, 2 times per week, 3 times per week, 4 times per week, 5 times per week, 6 times per week, 7 times per week, or more. A dose may be administered one time per month, two times per month, three times per month, four times per month, or more. A dose may be administered at one or more mealtimes. A dose may be administered immediately after a meal, about 0.5 hour after a meal, about 1 hour after a meal, about 2 hours after a meal, about 3 hours after a meal, about 4 hours after a meal, or about 5 hours after a meal. A dose may be administered immediately before a meal, about 0.5 hour before a meal, about 1 hour before a meal, about 2 hours before a meal, about 3 hours before a meal, about 4 hours before a meal, or about 5 hours before a meal. A dose may be administered within about 0.5 hour after a meal, within about 1 hour after a meal, within about 2 hours after a meal, within about 3 hours after a meal, within about 4 hours after a meal, or within about 5 hours after a meal. A dose may be administered within about 0.5 hour before a meal, within about 1 hour before a meal, within about 2 hours before a meal, within about 3 hours before a meal, within about 4 hours before a meal, or within about 5 hours before a meal.

A composition can be administered over the course of 1 day, 2 days, 3 days, 4 days, 5 days, 6 days, or 7 days. A composition can be administered over the course of about 2 weeks, about 3 weeks, about 4 weeks, about 5 weeks, about 6 weeks, about 7 weeks, about 8 weeks, about 9 weeks, about 10 weeks, about 11 weeks, or about 12 weeks. A composition can be administered over the course of about 3 months, about 4 months, about 5 months, about 6 months, about 7 months, about 8 months, about 9 months, about 10 months, about 11 months, or about 12 months. In some cases, a composition can be administered on an ongoing basis. An ongoing basis can be for a period of at least 1 year, at least 1.5 years, at least 2 years, at least 3 years, at least 4 years, at least 5 years, at least 6 years, at least 7 years, at least 8 years, at least 9 years, at least 10 years, or more.

A composition can have a shelf life of at least about: 1, 2, 3, 4, 5, 6, 7, 8, 9, 10, 11, or 12 months, or longer. In some cases, a composition can have a shelf life of at least about 1, 1.5, 2, 2.5, 3, 3.5, or 4 years, or longer. In some cases, a composition can have a shelf life of between 1 year and 4 years, between 1 year 3.5 years, between 1 year and 3 years, between 1 year and years, between 1 year and 2.5 years, between 1 year and 2 years, between 1.5 years and 4 years, between 1.5 years and 3.5 years, between 1.5 years and 3 years, between 1.5 years and 3 years, between 1.5 years and 2.5 years, between 1.5 years and 2 years, between 2 years and 4 years, between 2 years and 3.5 years, between 2 years and 3 years, between 2 years and 2.5 years, between 2.5 years and 4 years, between 2.5 years and 3.5 years, between 2.5 years and 3 years, between 3 years and 4 years, between 3 years and 3.5 years, or between 3.5 years and 4 years. In some cases, the shelf life as described here can apply to a composition which is stored at a refrigerated temperature. In some cases, the shelf life as described here can apply to a composition which is stored at room temperature. A composition comprising obligate anaerobic microbes may be formulated to reduce or eliminate the exposure to oxygen in order to increase shelf-life.

A composition disclosed herein may be formulated as a food or beverage product, cosmetic, or nutritional supplement. Microbial compositions can be formulated as a dietary supplement. Microbial compositions can be incorporated with vitamin supplements. Microbial compositions can be formulated in a chewable form such as a probiotic gummy. Microbial compositions can be incorporated into a form of food and/or drink. Non-limiting examples of food and drinks where the microbial compositions can be incorporated include, for example, bars, shakes, juices, infant formula, beverages, frozen food products, fermented food products, and cultured dairy products such as yogurt, yogurt drink, cheese, acidophilus drinks, and kefir. A composition disclosed herein can comprise microbes encased in a matrix. In some embodiments, a composition of the disclosure is a food form with microbes encased in almond butter.

A composition can be formulated for release to a suitable part of the gastrointestinal tract of a subject. Non-limiting examples of gastrointestinal tract regions include duodenum, small intestine regions including duodenum, jejunum, ileum, and large intestine regions including cecum, colon (e.g., ascending colon, transverse colon, descending colon, and/or sigmoid colon), rectum, and anal canal. The composition can be formulated for delivery to the ileum and/or colon regions of the gastrointestinal tract.

A composition can be formulated for delivery by any suitable delivery method. Non-limiting examples of delivery routes include topical, oral, parenteral, rectal, mucosal, vaginal, and enteral/gastrointestinal. A combination of administration routes can be utilized.

A composition can be administered orally, for example, as a capsule, pill, or tablet. A capsule, pill, or tablet of the disclosure can be a size 0 capsule, pill, or tablet. A capsule, tablet, or pill of the disclosure can be acid resistant. A capsule, tablet, or pill of the disclosure can comprise one or more of water, hydroxypropyl methylcellulose, hydroxypropyl methylcellulose phthalate, and propylene glycol. A capsule, tablet, or pill of the disclosure can comprise water, hydroxypropyl methylcellulose, hydroxypropyl methylcellulose phthalate, and propylene glycol. A capsule, tablet, or pill of the disclosure can comprise a casing consisting of or consisting essentially of hydroxypropyl methylcellulose, hydroxypropyl methylcellulose phthalate, and propylene glycol. A capsule, tablet, or pill of the disclosure can be a gelatin capsule with an average empty weight of 99 mg, a tolerance of +/−6 mg, and with the specifications disclosed in Table 2. A capsule, tablet, or pill of the disclosure can be a vegetarian capsule with an average empty weight of 105 mg, a tolerance of +/−6 mg, and with the specifications disclosed in Table 2.

TABLE 2
example capsule specifications
Empty capsule volume capacity

| | |
|---|---|
| Capacity | 0.68 |

Empty capsule weight capacity by powder density

| | |
|---|---|
| 0.6 g/mL | 408 |
| 0.8 g/mL | 544 |
| 1.0 g/mL | 680 |
| 1.2 g/mL | 816 |

Empty capsule overall closed length

| | |
|---|---|
| Millimeter (mm) | 21.6 |
| Tolerance | +/−0.3 |
| Inches (in) | 0.85 |
| Tolerance | +/−0.012 |

Empty capsule individual lengths (cap)

| | |
|---|---|
| Cap (mm) | 10.85 |
| Tolerance | +/−0.35 |
| Cap (inches) | 0.427 |
| Tolerance | +/−0.014 |

Empty capsule individual lengths (body)

| | |
|---|---|
| Body (mm) | 18.35 |
| Tolerance | +/−0.35 |
| Body (inches) | 0.772 |
| Tolerance | +/−0.014 |

Empty capsule external diameter

| | |
|---|---|
| Cap (mm) | 7.64 |
| Cap (inches) | 0.301 |
| Body (mm) | 7.35 |
| Body (inches) | 0.289 |

A composition can be administered orally, for example, through a capsule, pill, powder, tablet, gel, or liquid, designed to release the composition in the gastrointestinal tract.

In one non-limiting example, the microbial composition can be formulated for oral administration for example, in a pill or a capsule. The composition can comprise an enteric coating, for example, to prevent release of the contents in the stomach of the subject. The composition can be designed for a substantial release the composition contents in a gastrointestinal region of the subject, which can be a desired or preferred gastrointestinal region (e.g., duodenum, jejunum, ileum, cecum, upper colon, middle colon, lower colon, ascending colon, transverse colon, descending colon, sigmoid colon, rectum, anal canal, or a combination thereof).

An enteric-coating can protect the contents of a composition, for example, oral composition such as pill or capsule, from the acidity of the stomach. An enteric-coating can provide delivery to the ileum and/or upper colon regions. A microbial composition can be formulated such that the contents of the composition may not be released in a body part other than the gut region or preferred gut region, for example, ileum and/or colon region of the subject. Non-limiting examples of enteric coatings include pH sensitive polymers (e.g., eudragit FS30D), methyl acrylate-methacrylic acid copolymers, cellulose acetate succinate, hydroxy propyl methyl cellulose phthalate, hydroxy propyl methyl cellulose acetate succinate (e.g., hypromellose acetate succinate), polyvinyl acetate phthalate (PVAP), methyl methacrylate-methacrylic acid copolymers, shellac, cellulose acetate trimellitate, sodium alginate, zein, other polymers, fatty acids, waxes, shellac, plastics, and plant fibers. The enteric coating can be formed by a pH sensitive polymer. The enteric coating can be formed by eudragit FS30D.

The enteric coating can be designed to dissolve at any suitable pH. The enteric coating can be designed to dissolve at a pH greater than from about pH 6.5 to about pH 7.0. The enteric coating can be designed to dissolve at a pH greater than about pH 6.5. The enteric coating can be designed to dissolve at a pH greater than about pH 7.0. The enteric coating can be designed to dissolve at a pH greater than about: 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, or 7.5 pH units. The enteric coating can be designed to dissolve in the gut, for example, ileum and/or colon region. The enteric coating can be designed to not dissolve in the stomach.

Examples of formulations for probiotic delivery can comprise capsules, tablets, or beads. Additional parameters may be integrated into the compositions to increase the survival rate of the microbe.

The composition comprising one or more isolated and purified microbes, discussed herein may be encapsulated for delivery to a small intestine, a large intestine, an ileum, or a combination thereof, of the subject. The encapsulated mixture may not substantially release the population of isolated and purified microbes prior to a small intestine or a large intestine of the subject.

Solvent evaporation and cooling or crosslinking in a hardening bath may solidify air suspended droplets. Emulsification is another method that can involve the emulsification of a suspension or solutions of actives in continuous phase liquid. This can be followed by matrix/shell production by internal gelation, polymerization, layer by layer electrostatic deposition, internal phase separation, and coacervation. The common methods for solid shell and matrix formation in encapsulation processes can be mechanical and thermal, physicochemical, or chemical. Mechanical and thermal methods include cooling, freezing, pan coating, or fluidized-bed coating. Fluidizing bed coating can comprise top spray, bottom spray, tangential spray, or wurster process. Physicochemical methods may include solvent removal, layer by layer deposition, self-assembly, simple and complex coacervation, ionotropic gelation, or internal phase separation. Solvent removal includes evaporation or drying and liquid extraction. Chemical methods can comprise suspension polymerization, interfacial polycondensation, or sol-gel chemistry. Suspension polymerization may comprise one stage (direct) suspension polymerization or two-stage suspension polymerization (droplet swelling) method. Liposome can also be used for encapsulation.

Hydrogels can be used to encapsulate microbes. The microbes may comprise one or more strains. The hydrogels may comprise a hydrophilic active agent (e.g., one or more microbes of the present disclosure) that is captured in a hydrophilic polymer network. Chemical or physical gelation can form the gel networks. Chemical gelation may comprise the polymerization of free-radical processes or condensation. Physical gelation can make use of heating with heat setting gels, cooling with cold setting gels, or addition of multivalent counter ions via ionotropic gelation. Coacervation may comprise first an electrostatic phase separation in an emulsion or suspension of the active ingredient into a three phase system containing a polymer rich liquid phase, polymer lean liquid phase, and a liquid or solid phase with the active ingredient. Second, coacervation can comprise deposition of the coaverate phase onto the dispersed droplets or particles followed by a hardening of the coat.

In the solvent evaporation method, an organic solvent can dissolve a high melting point oil and the mixture is emulsified at room temperature with an aqueous phase. Next, the solid particles may be produced by organic solvent evaporation. As a result, the solid lipid particles are smaller than the initial oil droplets. On the other hand, during temperature-controlled emulsification, solid lipid microparticles can generally be the same size as the initial oil droplets. Hydrophilic samples can be encapsulated by forming a water in oil in water emulsion (W/O/W) prior to solvent evaporation or cooling.

The disclosure provides methods and compositions for treating a health condition, for example, a microbiome-associated health condition. Treatment can be achieved by, for example, administering a therapeutically-effective amount of a microbial-based composition at a suitable body site that shows a correlated link to disease onset. A composition can be delivered to the gut of a subject. An administered composition can be formulated for release in the gut of a subject.

Compositions disclosed herein can be used in a method to treat metabolic disorders. Non-limiting examples of metabolic disorders include prediabetes, diabetes, Type I diabetes mellitus, Type II diabetes mellitus, gestational diabetes, juvenile diabetes, metabolic syndrome, inflammatory bowel disease (IBD), irritable bowel syndrome (IBS), obesity, overweight condition, ischemia-reperfusion injury such as hepatic ischemia-reperfusion injury, fatty liver disease, non-alcoholic fatty liver disease (NAFLD), alcoholic steatohepatitis (ASH), non-alcoholic steatohepatitis (NASH), NAFLD in a non-obese subject (e.g., NAFLD not caused by or related to obesity or excess weight problems), NASH in a non-obese subject (e.g., NASH not caused or related to obesity or excess weight problems), Crohn's disease, colitis, ulcerative colitis, pseudomembranous colitis, renal dysfunction, nephrological pathology, glomerular disease, lactose intolerance, insulin insensitivity, insulin deficiency, insulin resistance, glucose intolerance, diarrhea, allergic diarrhea, dextran sodium sulfate-induced colitis, celiac disease, and gastroparesis. In some cases, the disorder can be type I diabetes. In some cases, the disorder can be type 2 diabetes. In some cases, the disorder can be prediabetes.

Compositions disclosed herein can be used in a method to manage, to alleviate, or to treat one or more metabolic disorders. In some cases, the one or more metabolic disorders treated can comprise type 2 diabetes. In some cases, the one or more metabolic disorders treated can comprise prediabetes or obesity.

A method for treating or managing type 2 diabetes can comprise administering to a subject with type 2 diabetes a probiotic composition comprising at least one butyrate-producing microbe and at least one mucin-regulating microbe, resulting in the reduction of hemoglobin A1C levels. Another method for treating or managing type 2 diabetes can comprise administering to a subject with type 2 diabetes a probiotic composition comprising at least one butyrate-producing microbe and at least one mucin-regulating microbe, resulting in a reduction in postprandial glucose levels following some meals, most meals, or all meals. Another method for treating or managing type 2 diabetes can comprise administering to a subject with type 2 diabetes a probiotic composition comprising at least one butyrate-producing microbe and at least one mucin-regulating microbe, resulting in the reduction of the glucose AUC after a meal tolerance test. Another method for treating or managing type 2 diabetes can comprise administering to a subject with type 2 diabetes a probiotic composition comprising at least one butyrate-producing microbe and at least one mucin-regulating microbe, resulting in the reduction of the fasting glucose level. In some methods which result in the reduction of hemoglobin AlC levels, the glucose AUC after a meal tolerance test can also be reduced. In some methods which result in the reduction of hemoglobin A1C levels, the fasting blood glucose can also be reduced. In some methods, the hemoglobin A1C level, the glucose AUC after a meal tolerance test, and the fasting glucose level can be reduced. Another method for treating or managing type 2 diabetes can comprise administering to a subject with type 2 diabetes a probiotic composition comprising at least one butyrate-producing microbe and at least one mucin-regulating microbe, resulting in the reduction of fasting glucose levels.

A method for treating or managing prediabetes can comprise administering to a subject with prediabetes a probiotic composition comprising at least one butyrate-producing microbe and at least one mucin-regulating-microbe, resulting in the reduction of hemoglobin A1C levels. Another method for treating or managing prediabetes can comprise administering to a subject with prediabetes a probiotic composition comprising at least one butyrate-producing microbe and at least one mucin-regulating microbe, resulting in a reduction in postprandial glucose levels following some meals, most meals, or all meals. Another method for treating or managing prediabetes can comprise administering to a subject with prediabetes a probiotic composition comprising at least one butyrate-producing microbe and at least one mucin-regulating microbe, resulting in the reduction of the glucose AUC after a meal tolerance test. Another method for treating or managing prediabetes can comprise administering to a subject with prediabetes a probiotic composition comprising at least one butyrate-producing microbe and at least one mucin-regulating microbe, resulting in the reduction of the fasting glucose level. In some methods which result in the reduction of hemoglobin A1C levels, the glucose AUC after a meal tolerance test can also be reduced. In some methods which result in the reduction of hemoglobin A1C levels, the fasting blood glucose can also be reduced. In some methods, the hemoglobin A1C level, the glucose AUC after a meal tolerance test, and the fasting glucose level can be reduced. Another method for treating or managing prediabetes can comprise administering to a subject with prediabetes a probiotic composition comprising at least one butyrate-producing microbe and at least one mucin-regulating microbe, resulting in the reduction of fasting glucose levels.

### Subjects

Subjects receiving a probiotic composition can have a diagnosis of type 2 diabetes. In some cases, subjects having a diagnosis of type 2 diabetes can have a fasting blood glucose level above 125 mg/dL. Subjects having a diagnosis of type 2 diabetes can have a blood glucose level after a glucose tolerance test above 199 mg/dL. Subjects having a diagnosis of type 2 diabetes can have a hA1C level which is greater than 6.4%. In some cases, subjects receiving a probiotic composition can have a diagnosis of prediabetes. In some cases, subjects having a diagnosis of prediabetes can have a fasting blood glucose level between 100 mg/dL and 125 mg/dL. In some cases, subjects having a diagnosis of prediabetes can have a blood glucose level after a glucose tolerance test between 140 mg/dL and 199 mg/dL. Subjects having a diagnosis of prediabetes can have a hA1C level between 5.7 and 6.4. Subjects having a diagnosis of type 2 diabetes can have early-stage, mid-stage, or late-stage diabetes.

Some subjects can have or have had increased hemoglobin A1C levels, increased glucose AUC after a meal tolerance test, or increased fasting glucose levels. Subjects can be currently or previously treated using other therapeutic agents, which can include insulin, metformin, sulfonylurea, or other medications. Subjects may receive a combination of the compositions disclosed herein and another therapeutic agent, e.g., insulin, metformin, a glucagon-like peptide 1 agonist (GLP-1), a gliptin, a sodium-glucose cotransporter-2 inhibitor (SGLT2), a meglitinide, an alpha-glucosidase inhibitor, and/or sulfonylurea.

Some subjects can have type 2 diabetes that may be well controlled. In some cases, subjects with well controlled diabetes can have a hemoglobin A1C level below 5.7% as measured from at least 30%, 40%, 50%, 60%, 70%, 80%, 90%, 95%, or 99% of their samples. In some cases, subjects with well controlled diabetes can have a fasting glucose level below 125 mg/dL as measured from at least 30%, 40%, 50%, 60%, 70%, 80%, 90%, 95%, or 99% of their samples. In some cases, patients with well controlled diabetes can have a fasting glucose level below 100 mg/dL as measured from at least 30%, 40%, 50%, 60%, 70%, 80%, 90%, 95%, or 99% of their samples. In some cases, patients with well controlled diabetes can have a postprandial glucose level below 110, 120, 130, 140, or 150 mg/dL.

Some subjects can be currently or previously on a restricted, controlled, or otherwise special diet. Some subjects may have had dietary intervention in the past. Some subjects may not have had dietary intervention.

Subjects can be administered a composition orally or nasogastrically. A composition can be solid, powdered, liquid, or encapsulated for delivery.

Subjects can have had type 2 diabetes for any amount of time. Subjects can have been diabetic for at least 1 month, 2 months, 3 months, 4 months, 5 months, 6 months, 7 months, 8 months, 9 months, 10 months, 11 months, 1 year, 2 years, 3 years, 4 years, 5 years, 10 years, 15 years, 20 years, 25 years, 30 years, or longer. In some embodiments, subjects can have been diabetic for at most 1 month, 2 months, 3 months, 4 months, 5 months, 6 months, 7 months, 8 months, 9 months, 10 months, 11 months, 1 year, 2 years, 3 years, 4 years, 5 years, 10 years, 15 years, 20 years, 25 years, 30 years, or less. Subjects can have had prediabetes for any amount of time. Subjects can have been prediabetic for at least 1 month, 2 months, 3 months, 4 months, 5 months, 6 months, 7 months, 8 months, 9 months, 10 months, 11 months, 1 year, 2 years, 3 years, 4 years, 5 years, 10 years, 15 years, 20 years, 25 years, 30 years, or longer. In some embodiments, subjects can have been prediabetic for at most 1 month, 2 months, 3 months, 4 months, 5 months, 6 months, 7 months, 8 months, 9 months, 10 months, 11 months, 1 year, 2 years, 3 years, 4 years, 5 years, 10 years, 15 years, 20 years, 25 years, 30 years, or less.

Subjects can have an additional comorbidity. Additional comorbidities can include preterm labor, chronic fatigue syndrome, skin conditions such as acne, allergies, autism, asthma, depression, hypertension, irritable bowel syndrome, metabolic syndrome, obesity, lactose intolerance, oral thrush, ulcerative colitis, drug metabolism, vaginosis, atopic dermatitis, psoriasis, Multiple Sclerosis, neurological disorders such as Parkinson's disease, Clostridium difficile infection, Inflammatory Bowel Disease, Crohn's Disease, heart disease, diabetic foot ulcers, bacteremia, infantile colic, cancer, cystic fibrosis, multiple sclerosis, urinary tract infection, radiation enteropathy, drug metabolism, dental cavities, halitosis, metabolic disorder, gastrointestinal disorder, insulin insensitivity, metabolic syndrome, Non-Alcoholic Fatty Acid Liver Disease (NAFLD), Nonalcoholic steatohepatitis (NASH), Cardiovascular Disease, Hypertension, disorder associated with Cholesterol, disorder associated with Triglycerides, obesity, overweight condition, inflammation, infant formula feeding, appendicitis, atopic disease, ageing, fasting, pregnancy, obesity during pregnancy, dextran sodium sulfate-induced colitis, diarrhea, allergic diarrhea, and atherosclerosis.

In some embodiments, a subject can be a healthy subject. For example, a composition or method of the disclosure can be used to prevent, delay, or decrease the risk of a healthy subject developing prediabetes or type 2 diabetes. In some embodiments, a composition of the disclosure can be administered to a subject with a hA1C level of less than 5.7. A composition of the disclosure can be administered to a subject with a glucose AUC after MTT of between 14,500 mg min/dL and 22,000 mg min/dL. A composition of the disclosure can be administered to a subject with a blood glucose level after a glucose tolerance test less than 140 mg/dL. A composition of the disclosure can be administered to a subject with a fasting glucose level of less than 100 mg/dL. A composition of the disclosure can be administered to a subject with a postprandial glucose level of less than 140 mg/dL. A composition of the disclosure can be administered to a subject that does not show signs or symptoms of prediabetes or type 2 diabetes. A composition of the disclosure can be administered to a subject that has not undergone clinical testing for prediabetes or type 2 diabetes.

### Treatment Plans

Probiotic compositions can be administered to subjects as part of a treatment plan. A treatment plan can include additional therapies, a special diet, an exercise regime, bariatric surgery, or other treatments or lifestyle changes. If a treatment plan includes additional therapies, these can be formulated with a composition, co-administered with a composition, or administered separately from a composition or at a different time than the composition, as described below.

Treatment plans can include administering the probiotic composition daily. Daily administration can include administering a composition one, two, three, four, five, or more times per day. Treatment plans can include administering the probiotic composition one, two, three, four, five, six, seven, eight, nine, 10, 11, 12, 13, 14, 15, 20, 25, 30, 35, 40, 45, 50, 60, 70, 80, 90, 100, 110, 120, 130, 140, 150, 175, 200, 250, 300, 350, 365, 400, 500, 600, 700, 800, 900, 1000, or more times total. Treatment plans can include administering the probiotic composition every one, two, three, four, five, six, seven, or more days. Treatment plans can include administering the probiotic composition weekly, biweekly, monthly, bimonthly, quarterly, semiannually, or annually. Some treatment plans may require regularly scheduled dosing. Some treatment plans may allow for or require irregularly scheduled dosing. Some treatment plans may require testing to determine when additional probiotic composition should be administered. As an example, stool samples can be monitored, and a composition can be administered to a subject if the amounts of administered probiotic in the subject's microbiome drop below a specified level.

Some compositions can be administered over at least 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks. Some compositions can be administered for up to 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 26 weeks, 1 year, 2 years, 3 years, 4 years, or 5 years. Some compositions can be administered for about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 26 weeks, 1 year, 2 years, 3 years, 4 years, or 5 years.

A probiotic composition can be administered before, during, or after treatment with an antimicrobial agent such as an antibiotic. The probiotic composition can be administered at least about 1 hour, 2 hours, 5 hours, 12 hours, 1 day, 2 days, 3 days, 4 days, 5 days, 6 days, 1 week, 8 days, 9 days, 10 days, 11 days, 12 days, 13 days, 2 weeks, 2.5 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 6 months, or 1 year before and/or after treatment with an antibiotic. The probiotic composition can be administered at most 1 hour, 2 hours, 5 hours, 12 hours, 1 day, 2 days, 3 days, 4 days, 5 days, 6 days, 1 week, 8 days, 9 days, 10 days, 11 days, 12 days, 13 days, 2 weeks, 2.5 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 6 months, or 1 year before and/or after treatment with an antibiotic.

In some cases, the probiotic composition can administered before, during, or after food consumption as disclosed herein.

### Metformin

In some cases, a composition can be administered with metformin. In some cases, a composition of the disclosure can be provided as a combination therapy with metformin. Metformin can be administered at the same time as a composition, or can be administered 1, 2, 3, 4, 5, 6, 7, 8, 9, 10, 11, or 12 hours before or after administering a composition.

Metformin can be formulated in the composition or administered separately. In some cases, a patient can be on a metformin regimen already when they begin administration of the probiotic composition. In some cases, a patient can begin metformin therapy at the same time as they begin administration of the composition. In some cases, a patient can begin metformin therapy after they begin administration of the composition.

Metformin can be administered at a dose of about 500 mg, about 550 mg, about 600 mg, about 650 mg, about 700 mg, about 750 mg, about 800 mg, about 850 mg, about 900 mg, about 950 mg, or about 1000 mg. Metformin can be administered at least once or twice per day. In some cases, the metformin dose may be at least 500 mg per day. In some cases, the metformin dose may not be more than 2500 mg per day. In some cases the metformin dose may between 500 mg and 2500 mg per day.

In some cases, administered metformin can increase the therapeutic effect of the administered composition. An increased or enhanced therapeutic effect in a subject suffering from a metabolic disorder may be shown by a greater reduction in the hemoglobin A1C level than occurs from the administration of the composition alone. In some cases, an increased or enhanced therapeutic effect in a subject suffering from a metabolic disorder may be shown by a greater reduction in glucose AUC after MTT than occurs from the administration of the composition alone. In some cases, an increased or enhanced therapeutic effect in a subject suffering from a metabolic disorder may be shown by a greater reduction in the fasting glucose level than occurs from the administration of the composition alone.

In some cases, the administered composition can increase the therapeutic effect of the administered metformin. An increased or enhanced therapeutic effect in a subject suffering from a metabolic disorder may be shown by a greater reduction in the hemoglobin A1C level than occurs from the administration of metformin alone. In some cases, an increased or enhanced therapeutic effect in a subject suffering from a metabolic disorder may be shown by a greater reduction in glucose AUC after MTT than occurs from the administration of metformin alone. In some cases, an increased or enhanced therapeutic effect in a subject suffering from a metabolic disorder may be shown by a greater reduction in the fasting glucose level than occurs from the administration of metformin alone.

In some cases, when co-administered with a composition of the disclosure or administered as part of a composition of the disclosure, metformin can be administered at a dose which would be subtherapeutic if metformin were administered alone. In some cases, a subtherapeutic dose of metformin may be a dose of metformin which does not reduce the hemoglobin A1C level or does not reduce the hemoglobin A1C level to at least a threshold level. In some cases, a subtherapeutic dose of metformin may be a dose of metformin which does not reduce the glucose AUC after MTT, or does not reduce the glucose AUC after MTT to at least a threshold level. In some cases, a subtherapeutic dose of metformin may be a dose of metformin which does not reduce the fasting glucose level, or does not reduce the fasting glucose level to at least a threshold level. In some cases, such a subtherapeutic dose of metformin can enhance the therapeutic effect of the composition. In some cases, the composition can enhance the effect of a dose of metformin, subtherapeutic or otherwise.

### Sulfonylurea

In some cases, the composition can be administered in the absence of sulfonylurea. In particular, for some subjects, it may be desirable to administer the compositions described herein in the absence of co-administration with sulfonylurea. Accordingly, the compositions described herein may be administered to subjects who are not being administered sulfonylureas at the same time as they are receiving the compositions described herein. Such subjects for treatment may be identified as those not currently being treated with sulfonylurea, or may be instructed to stop taking sulfonylurea prior to beginning administration of the compositions described herein.

In some cases, the composition can be administered with a low dose of sulfonylurea. The low dose can be administered concurrently, before, or after administration of a microbial composition of the disclosure. A low dose may be a subtherapeutic dose, which can be a dose which may not provide a therapeutic effect when sulfonylurea is administered alone. In some cases, sulfonylurea can be administered at a reduced dose of less than about 0.25, 0.5, 1, 1.25, 1.5, 1.75, 2, 2.25, or 2.5 mg.

In some cases, an appropriate dosage or treatment regimen of sulfonylurea can be determined experimentally. For example, subjects can be divided into groups, each given a therapeutically effective dose of a composition described herein and a dose of sulfonylurea. Examples of subjects can include humans, mice, or rats. In some cases, each group can receive a different dose of sulfonylurea. In some cases, one or more groups can receive a subtherapeutic dose of sulfonylurea.

In other cases, sulfonylurea administration and administration of the compositions described herein, may be staggered, such that their administration to a patient is separated by at least 1 day, at least 2 days, at least 3 days, at least 4 days, at least a week, or longer.

In Combination with Insulin Therapy

Some probiotic compositions herein can be administered to a subject who is receiving insulin therapy, or to a subject who has received insulin therapy in the past. In some cases, probiotic compositions can be administered to a subject at the same time as insulin. In some cases, administered insulin can improve the therapeutic effect of the administered probiotic composition. In some cases, insulin therapy can be used as needed. In some cases, insulin may be taken alongside or at around the same time as a composition comprising at least one isolated and purified butyrate-producing microbe and at least one isolated and purified mucin-regulating microbe. In some cases, at least 0.5, 1, 2, 3, or 4 hours can elapse prior to administering insulin. In some cases, the administered probiotic composition can reduce or eliminate a subject's need for insulin.

In Combination with Other Therapies

In some cases, a probiotic composition described herein can be administered along with another therapy or therapies. Examples of other therapies can include meglitinides, thiazolidinediones, DPP-4 inhibitors, GLP-1 receptor agonists, or SGLT2 inhibitors. One or more other therapies can increase or enhance the therapeutic effect of the administered composition comprising at least one isolated and purified butyrate-producing microbe and at least one isolated and purified mucin-regulating microbe. An increased or enhanced therapeutic effect in a subject suffering from a metabolic disorder may be shown by a greater reduction in the hemoglobin A1C level than occurs from the administration of the composition alone. In some cases, the hemoglobin A1C can be reported as a percentage of hemoglobin which has been glycated or glycosylated. In some cases, hemoglobin A1C can be reduced by an additional 1%, 2%, 3%, 4%, 5%, 6%, 7%, 8%, 9%, 10%, 11%, 12%, 13%, 14%, or 15% when the composition is administered with another therapy. In some cases, hemoglobin A1C can be reduced by an additional 0.05%, 0.06%, 0.07%, 0.08%, 0.09%, 0.1%, 0.2%, 0.3%, 0.4%, 0.55%, 0.6%, 0.7%, 0.8%, 0.9%, or 1% of total hemoglobin when the composition is administered with another therapy. In some cases, an increased or enhanced therapeutic effect in a subject suffering from a metabolic disorder may be shown by a greater reduction in glucose AUC after MTT than occurs from the administration of the composition alone. In some cases, the glucose AUC after MTT can be reduced by an additional 1%, 2%, 3%, 4%, 5% 10%, 15%, 20%, 25%, 30%, 35%, 40%, 45%, or 50% when the composition is administered with another therapy. In some cases, an increased or enhanced therapeutic effect in a subject suffering from a metabolic disorder may be shown by a greater reduction in the fasting glucose level than occurs from the administration of the composition alone. In some cases, the fasting glucose level can be reduced by an additional 1%, 2%, 3%, 4%, 5%, 10%, 15%, 20%, 25%, 30%, 35%, 40%, 45%, or 50% when the composition is administered with another therapy.

Some subjects with type 2 diabetes can undergo bariatric surgery in effort to control the condition. In some cases, a probiotic composition can be administered to a subject before or after bariatric surgery. Without being limited by theory, bariatric surgery may improve the therapeutic effect of the administered probiotic composition.

In Combination with a Special Diet or Dietary Regime

In some cases, a probiotic composition described herein can be administered alongside a special diet or dietary regime. For example, a subject can consume a diet high in one or more of fruits, vegetables, whole grains. In another example, a subject may consume a diet limited in one or more of animal products, refined carbohydrates, or sweets. Some special diets or dietary regimens can limit foods with a high glycemic index. Some special diets or dietary regimens can comprise a large amount of foods with a low glycemic index.

Some special diets can include a low glycemic index diet, a vegetarian diet, a vegan diet, a semi-vegetarian diet, a low-calorie diet, a low-carbohydrate diet, a low-fat diet, a gluten free diet, a sugar free diet, a low-sugar diet, a low-glucose diet, a ketogenic diet, a liquid diet, a low-FODMAP (Fermentable Oligosaccharides, Disaccharides, Monosaccharides and Polyols) diet, a specific carbohydrate diet, a diet free of one or more allergens, or another type of diet. Some diets can control or aid in controlling type 2 diabetes on their own. In some cases, a special diet can improve the therapeutic effect of an administered probiotic composition. Some diets can control or aid in controlling type 2 diabetes when combined with a probiotic composition described herein.

In Combination with an Exercise Regime

Some subjects with type 2 diabetes may begin, continue, or change an exercise regime. Probiotic compositions herein can be administered to these subjects, or to subjects on any exercise regime or no exercise regime. In some cases, an exercise regime can improve the therapeutic effect of the administered probiotic composition.

### Outcomes

Administering a probiotic composition described herein can result in one or more outcomes. These outcomes can be achieved when the probiotic composition is administered alone, with another therapeutic, in combination with a special diet, in combination with bariatric surgery, or in combination with an exercise regime. Outcomes can be independent of whether a probiotic composition is administered alone, with another therapeutic, in combination with a special diet, in combination with bariatric surgery, or in combination with an exercise regime, or in combination with a combination of the above. For example, outcomes can be achieved when the probiotic composition is administered in combination with a special diet and exercise in a subject which has undergone bariatric surgery. In some cases, outcomes can be dependent on administering a probiotic composition alone, with another therapeutic, in combination with a special diet, in combination with bariatric surgery, or in combination with an exercise regime.

Outcomes can be changed for all subjects or for a subset of subjects. Some subsets of subjects can have different outcomes than other subsets of subjects. As such, some subsets of subjects can have different outcomes than the average of outcomes of all subjects, and some subsets of subjects can have different outcomes than a typical outcome for subjects.

When outcomes are described herein, for example a change or percent change, they can be for an individual, a subset of subjects, for all subjects, the average outcome, the median outcome, the mode outcome, or an expected range of outcomes. An outcome can be determined on a per-subject basis, for example, a change in a parameter for a subject that is calculated by comparing the parameter before administering a composition of the disclosure to after administering the composition. An outcome can be determined relative to a control. A control can be a parameter measured in the subject before administering a composition of the disclosure. A control can be a parameter measured in a subject that is not administered a composition of the disclosure. A control can be a parameter measured in a subject that is administered a placebo. A control can be a parameter measured in a subject that is administered an alternate composition or an alternate therapeutic.

An outcome can inform the measurement or detection of a therapeutic effect. In some cases, an outcome can be a therapeutic effect. In some cases, a therapeutic effect can be a reduction in the hemoglobin AIC level after administration of the composition. In some cases, a therapeutic effect can be a reduction in glucose AUC after MTT following administration of the composition. In some cases, a therapeutic effect can be a reduction in fasting glucose level after administration of the composition.

In some cases, a reduction in meal related glucose AUC can be demonstrated in a free living situation. For example, such a reduction in meal related glucose AUC can be observed via continuous glucose monitoring or frequent glucose monitoring.

Reduction of hA1C

hA1C can be measured from a volume of blood. In some cases, measurement of hA1C can be an indicator of type 2 diabetes or prediabetes. In some cases, the hemoglobin AIC can be reported or measured as a percentage of hemoglobin which is glycosylated. The percentage of glycosylated hemoglobin can indicate the average blood glucose level over approximately the past three months. For example, low hA1C levels can indicate the absence or control of prediabetes or diabetes in a subject.

Changes in hA1C levels can occur within about 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 22 weeks, 24 weeks, or 26 weeks of beginning an intervention (e.g., after beginning to take a composition of the disclosure). Changes in hA1C levels can occur about 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 22 weeks, 24 weeks, or 26 weeks after beginning an intervention (e.g., after beginning to take a composition of the disclosure).

hA1C levels can vary between subjects or in the same subjects over time. In some cases, hA1C levels can be indicative of whether a subject has prediabetes or diabetes. For example, an hA1C level below 5.7% can be indicative of a non-diabetic subject or a subject whose diabetes or prediabetes is well controlled on a therapy or diet regime or exercise regime or combination thereof. In some subjects, an hA1C level of between 5.7% and 6.4% can be indicative of prediabetes. In some cases, an hA1C level at or above 6.5% can be indicative of diabetes, including type 2 diabetes.

hA1C can be measured multiple times in some subjects. The average, median, mode, highest, lowest, most recent level, change in level, or a combination thereof can be considered.

hA1C can be measured in a volume of blood which is at least 0.6, 0.7, 0.8, 0.9, 1.0, 1.5, 2.0, 2.5, 3.0, 3.5, 4.0, 4.5, or 5 or more μL of blood. The blood volume used to measure the hA1C level can be at least 1 μL, 5 μL, 10 μL, 15 μL, 20 μL, 30 μL, 40 μL, 50 μL, 100 μL, 200 μL, 300 μL, 400 μL, 500 μL, 600 μL, 700 μL, 800 μL, 900 μL, 1 mL, 2 mL, 3 mL, 4 mL, 5 mL, 6 mL, 7 mL, 8 mL, 9 mL, or 10 mL of blood, or more.

In some cases, administering a composition as disclosed herein can reduce the hA1C by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, or 50% of the pre-treatment hA1C level. In some cases, administering a composition as disclosed herein can reduce the hA1C by between 1% and 50%, between 1%, and 40%, between 1% and 30%, between 1% and 20%, between 1% and 10%, between 5% and 50%, between 5% and 40%, between 5% and 30%, between 5% and 20%, between 5% and 10%, between 10% and 50%, between 10% and 40%, between 10% and 30%, between 20% and 50%, between 20% and 40%, between 20% and 30%, between 30% and 50%, between 30% and 40%, or between 40% and 50%. In some cases, final hA1C can be about 3%, about 4%, about 5%, about 6%, or about 7% of total hemoglobin. As an illustrative example, a 10% hA1C reduction in a subject having an hA1C level of 7% of total hemoglobin, which can be indicative of diabetes, can result in a final hA1C level of 6.3% of total hemoglobin, which can be indicative of prediabetes.

In some cases, administering a composition as disclosed herein can reduce an hA1C level by at least 0.05, 0.1, 0.2, 0.3, 0.4, 0.5, 0.6, 0.7, 0.8, 0.9, 1.0, 1.1, 1.2, 1.3, 1.5, 1.6, 1.7, 1.8, 1.9, 2, 2.1, 2.2, 2.3, 2.4, 2.5, 2.6, 2.7, 2.8, 2.9, or 3.0% of total hemoglobin, or more. In some cases, administering a probiotic composition disclosed herein can reduce the hA1C level by between about 0.05% and about 3%, between about 0.05% and about 2.5%, between about 0.05% and about 2%, between about 0.05% and about 1.5%, between about 0.05% and about 1.2%, between about 0.05% and about 1.1%, between about 0.05% and about 1%, between about 0.05% and about 0.9%, between about 0.05% and about 0.8%, between about 0.05% and about 0.7%, between about 0.05% and about 0.6%, between about 0.05% and about 0.5%, between about 0.05% and about 0.4%, between about 0.05% and about 0.3%, between about 0.05% and about 0.2%, between about 0.05% and about 1%, between about 0.1% and about 2.5%, between about 0.1% and about 2%, between about 0.1% and about 1.5%, between about 0.1% and about 1.2%, between about 0.1% and about 1.1%, between about 0.1% and about 1%, between about 0.1% and about 0.9%, between about 0.1% and about 0.8%, between about 0.1% and about 0.7%, between about 0.1% and about 0.6%, between about 0.1% and about 0.5%, between about 0.1% and about 0.4%, between about 0.1% and about 0.3%, between about 0.1% and about 0.2%, between about 0.2% and about 2.5%, between about 0.2% and about 2%, between about 0.2% and about 1.5%, between about 0.2% and about 1.2%, between about 0.2% and about 1.1%, between about 0.2% and about 1%, between about 0.2% and about 0.9%, between about 0.2% and about 0.8%, between about 0.2% and about 0.7%, between about 0.2% and about 0.6%, between about 0.2% and about 0.5%, between about 0.2% and about 0.4%, between about 0.2% and about 0.3%, between about 0.3% and about 2.5%, between about 0.3% and about 2%, between about 0.3% and about 1.5%, between about 0.3% and about 1.2%, between about 0.3% and about 1.1%, between about 0.3% and about 1%, between about 0.3% and about 0.9%, between about 0.3% and about 0.8%, between about 0.3% and about 0.7%, between about 0.3% and about 0.6%, between about 0.3% and about 0.5%, between about 0.3% and about 0.4%, between about 0.4% and about 2.5%, between about 0.4% and about 2%, between about 0.4% and about 1.5%, between about 0.4% and about 1.2%, between about 0.4% and about 1.1%, between about 0.4% and about 1%, between about 0.4% and about 0.9%, between about 0.4% and about 0.8%, between about 0.4% and about 0.7%, between about 0.4% and about 0.6%, between about 0.4% and about 0.5%, between about 0.5% and about 2.5%, between about 0.5% and about 2%, between about 0.5% and about 1.5%, between about 0.5% and about 1.2%, between about 0.5% and about 1.1%, between about 0.5% and about 1%, between about 0.5% and about 0.9%, between about 0.5% and about 0.8%, between about 0.5% and about 0.7%, between about 0.5% and about 0.6%, between about 0.5% and about 0.5%, between about 0.5% and about 0.4%, between about 0.6% and about 1.1%, between about 0.6% and about 1%, between about 0.6% and about 0.9%, between about 0.6% and about 0.8%, or between about 0.6% and about 0.7%, of total hemoglobin. In some cases, a probiotic composition disclosed herein can reduce the hA1C level by between about 0.4% and about 0.7% of total hemoglobin. In some cases, administering a probiotic composition disclosed herein can reduce the hA1C level by between about 0.55 and about 0.65% of total hemoglobin. The percent change can be relative to pre-administration levels, or relative to a control subject that is administered a placebo and/or is not administered the same composition.

In some cases, the composition can be administered over a course of time, for example, days or weeks, and the hA1C levels can undergo reduction over that course of administration as determined by measuring the hA1C levels at various time points during the length of treatment. For example, in some cases, the composition can be administered for at least 12 weeks. In some cases, after 6 months, the hA1C can be reduced by an average of at least 0.1, an average of at least 0.2, by an average of at least 0.3, an average of at least 0.4, by an average of at least 0.5, or an average of at least 0.6% of total hemoglobin. In some cases, after 12 weeks, the hA1C can be reduced by an average of at least 0.1, an average of at least 0.2, by an average of at least 0.3, an average of at least 0.4, by an average of at least 0.5, or an average of at least 0.6% of total hemoglobin. In some cases, after 10 weeks, the hA1C can be reduced by at least 5%, 10%, or 15% relative to the starting hA1C level or relative to a control subject. In some cases, after 8 weeks, the hA1C can be reduced by an average of at least 0.1, an average of at least 0.2, an average of at least 0.3, an average of at least 0.4, an average of at least 0.5, or an average of at least 0.6% of total hemoglobin. In some cases, after 6 weeks, the hA1C can be reduced by an average of at least 0.05, an average of at least 0.1, an average of at least 0.2, an average of at least 0.3, an average of at least 0.4, an average of at least 0.5, or an average of at least 0.6% of total hemoglobin. In some cases, after 4 weeks, the hA1C can be reduced by an average of at least 0.05, an average of at least 0.1, an average of at least 0.2, an average of at least 0.3, an average of at least 0.4, an average of at least 0.5, or an average of at least 0.6% of total hemoglobin. In some cases, the reduction in hA1C over a defined time period may not exceed the reduction in hA1C over a shorter defined time period.

In some cases, administering a composition as disclosed herein can reduce the hA1C level in at least 5%, 10%, 20%, 30%, 40%, 50%, 60%, 70%, 80%, 90%, 95%, or 99% of subjects having prediabetes or type 2 diabetes.

In some cases, the hA1C levels can be reduced from a diabetic level to a normal level. In some cases, the hA1C levels can be reduced from a diabetic level to a pre-diabetic level. In some cases, the hA1C levels can be reduced from a pre-diabetic level to a normal level. In some cases, the hA1C levels can be reduced from a higher diabetic level to a lower diabetic level. In some cases, the hA1C levels can be reduced from a higher pre-diabetic level to a lower pre-diabetic level.

Reduction of Glucose AUC after MTT

Herein, “MTT” can refer to a meal tolerance test. An MTT can comprise administering a meal which can be a standardized meal followed by measuring glucose in time separated blood samples. A meal which can be a standardized meal can comprise a standardized nutritional food, such as a liquid nutritional shake, such as Ensure®, or other similar standardized high nutrient content foods. In some cases, glucose can be measured using a continuous glucose monitor. In some cases a subject may not eat or drink before the MTT. In some cases, blood samples may be taken via a catheter, syringe, or other method. Blood samples may be taken about every 10, 15, 20, 25, 30, 45, or 60 minutes, or at any other interval. Blood samples may be taken over the course of about 1, 1.5, 2, 2.5, 3, 3.5, 4, 4.5, or 5 hours, or over another appropriate duration for the subject. In some cases, blood samples may be taken until the blood glucose level is that of fasting blood glucose.

Changes in glucose AUC after MTT levels can occur within about 1 day, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 22 weeks, 24 weeks, or 26 weeks of beginning an intervention (e.g., after beginning to take a composition of the disclosure).

Changes in glucose AUC after MTT levels can occur about 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 22 weeks, 24 weeks, or 26 weeks after beginning an intervention (e.g., after beginning to take a composition of the disclosure).

Glucose AUC after MTT can vary between subjects or in the same subject over time. In some subjects, a healthy glucose AUC after MTT can be between 14,500 mg min/dL and 22,000 mg min/dL.

Glucose AUC after MTT can be measured after a random meal, after a meal high in sugar, after a meal low in sugar, after a large meal, after a small meal, after an average meal, after a standardized meal, after a prescribed meal, or after a dietary or meal replacement supplement.

Glucose AUC after MTT can be measured multiple times in some subjects. The average, median, mode, highest, lowest, or most recent level, or a combination thereof, can be considered.

Glucose AUC after MTT can be measured from serially obtained blood samples. Glucose AUC after MTT can be measured using 2, 3, 4, 5, 6, 7, 8, 9, 10, or more time points. For each time point, the blood volume used to measure the glucose level can be at least 0.5, 0.6, 0.7, 0.8, 0.9, 1.0, 1.5, 2.0, 2.5, 3.0, 3.5, 4.0, 4.5, or 5 or more μL of blood. For each time point, the blood volume used to measure the glucose level can be at least 1 μL, 5 μL, 10 μL, 15 μL, 20 μL, 30 μL, 40 μL, 50 μL, 100 μL, 200 μL, 300 μL, 400 μL, 500 μL, 600 μL, 700 μL, 800 μL, 900 μL, 1 mL, 2 mL, 3 mL, 4 mL, 5 mL, 6 mL, 7 mL, 8 mL, 9 mL, or 10 mL of blood, or more.

In some cases, glucose AUC after MTT can be measured without drawing blood samples, for example, using a continuous glucose monitoring device.

In some cases, administering a composition as disclosed herein can reduce the glucose AUC after MTT by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, or 75% relative to a glucose AUC after MTT measured before administering the composition, or relative to a control subject that is not administered the composition. In some cases, administering a probiotic composition herein can reduce the glucose AUC after MTT by between 12% and 18%. In some cases, a probiotic composition can reduce the glucose AUC after MTT by between 10% and 20%. In some cases, a probiotic composition can reduce the glucose AUC after MTT by between 5% and 25%. In some cases, a probiotic composition can reduce the glucose AUC after MTT by between 5% and 30%. In some cases, administering a composition as disclosed herein can reduce the glucose AUC after MTT by between 1% and 50%, between 1%, and 40%, between 1% and 30%, between 1% and 20%, between 1% and 10%, between 5% and 50%, between 5% and 40%, between 5% and 30%, between 5% and 20%, between 5% and 10%, between 10% and 50%, between 10% and 40%, between 10% and 30%, between 20% and 50%, between 20% and 40%, between 20% and 30%, between 30% and 50%, between 30% and 40%, or between 40% and 50%.

In some cases, administering a probiotic composition herein can reduce the glucose AUC after MTT in at least 5%, 10%, 20%, 30%, 40%, 50%, 60%, 70%, 80%, 90%, 95%, or 99% of subjects having prediabetes or type 2 diabetes.

In some cases, glucose AUC level after MTT is reduced by at least about 4.5, 5, 5.5, 6, 6.5, or 7 percentage points. The change can be relative to pre-administration levels, or relative to a control subject that is administered a placebo and/or is not administered the same composition.

In some cases, the composition can be administered over a course of time, for example, days or weeks, and the glucose levels can undergo reduction over that course of administration as determined by measuring the glucose levels at various time points during the length of treatment.

For example, in some cases, the composition can be administered for at least 12 weeks. In some cases, after 6 months, the glucose AUC can be reduced by an average of at least 5%, 10%, 15%, 20%, 25%, 30%, 35%, 40%, 50%, or 60% relative to pre-administration levels, or relative to a control subject that is administered a placebo and/or is not administered the same composition. In some cases, after 12 weeks, the glucose AUC can be reduced by an average of at least 5%, 10%, 15%, 20%, 25%, 30%, 35%, 40%, 50%, or 60% relative to pre-administration levels, or relative to a control subject that is administered a placebo and/or is not administered the same composition. In some cases, after 10 weeks, the glucose AUC after MTT can be reduced by an average of at least 5%, 10%, 15%, 20%, 25%, 30%, 35%, 40%, 50%, or 60% relative to pre-administration levels, or relative to a control subject that is administered a placebo and/or is not administered the same composition. In some cases, after 8 weeks, the glucose AUC after MTT can be reduced by an average of at least 5%, 10%, 15%, 20%, 25%, 30%, 35%, 40%, 50%, or 60% relative to pre-administration levels, or relative to a control subject that is administered a placebo and/or is not administered the same composition. In some cases, after 6 weeks, the glucose AUC after MTT can be reduced by an average of at least 5%, 10%, 15%, 20%, 25%, 30%, 35%, 40%, 50%, or 60% relative to pre-administration levels, or relative to a control subject that is administered a placebo and/or is not administered the same composition. In some cases, after 4 weeks, the glucose AUC after MTT can be reduced by an average of at least 5%, 10%, 15%, 20%, 25%, 30%, 35%, 40%, 50%, or 60% relative to pre-administration levels, or relative to a control subject that is administered a placebo and/or is not administered the same composition.

In some cases, the glucose AUC after MTT can be reduced from a diabetic level to a normal level. In some cases, the glucose AUC after MTT can be reduced from a diabetic level to a pre-diabetic level. In some cases, the glucose AUC after MTT can be reduced from a pre-diabetic level to a normal level. In some cases, the glucose AUC after MTT can be reduced from a higher diabetic level to a lower diabetic level. In some cases, the glucose AUC after MTT can be reduced from a higher pre-diabetic level to a lower pre-diabetic level.

In some cases, the reduction in AUC after MTT over a defined time period may not exceed the reduction in AUC after MTT over a shorter defined time period.

### Reduction of Fasting Glucose Levels

Fasting glucose levels can vary between subjects or in the same subject overtime. In some subjects, a healthy fasting glucose level can be less than 100 mg/dL. In some subjects, a fasting glucose level indicative of prediabetes can be between 100 and 125 mg/dL. In some subjects, a fasting glucose level indicative of diabetes can be 125 mg/dL or more.

Changes in fasting glucose levels can occur within about 1 day, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 22 weeks, 24 weeks, or 26 weeks of beginning an intervention (e.g., after beginning to take a composition of the disclosure).

Changes in glucose fasting glucose levels can occur about 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 22 weeks, 24 weeks, or 26 weeks after beginning an intervention (e.g., after beginning to take a composition of the disclosure).

Fasting glucose levels can be the amount of glucose measured in a blood sample a significant period of time after food is consumed. Fasting glucose can be measured after an overnight fast or after waking from sleep. Fasting glucose can be measured about 3, 4, 5, 6, 7, 8, 9, 10, 11, 12, or 13 hours or longer hours after food is consumed. Fasting glucose can be measured at least 3, 4, 5, 6, 7, 8, 9, 10, 11, 12, or 13 hours after food is consumed.

Fasting glucose can be measured multiple times in some subjects. The average, median, mode, highest, lowest, or most recent level can be considered.

Fasting glucose can be measured in a volume of blood which is at least 0.4, 0.5, 0.6, 0.7, 0.8, 0.9, 1.0, 1.5, 2.0, 2.5, 3.0, 3.5, 4.0, 4.5, or 5 or more μL of blood. The blood volume used to measure the glucose level can be at least 1 μL, 5 μL, 10 μL, 15 μL, 20 μL, 30 μL, 40 μL, 50 μL, 100 μL, 200 μL, 300 μL, 400 μL, 500 μL, 600 μL, 700 μL, 800 μL, 900 μL, 1 mL, 2 mL, 3 mL, 4 mL, 5 mL, 6 mL, 7 mL, 8 mL, 9 mL, or 10 mL of blood, or more.

In some cases, fasting glucose can be measured without drawing blood samples, for example, using a continuous glucose monitoring device.

Fasting glucose levels can be measured multiple times in some subjects. The average, median, mode, highest, lowest, most recent level, or a combination thereof can be considered.

In some cases, administering a composition as disclosed herein can reduce the fasting glucose level by at least 1%, 2%, 3%, 4%, 5%, 6%, 7% 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, or 80% relative to pre-administration levels, or relative to a control subject that is administered a placebo and/or is not administered the same composition. In some cases, administering a composition as disclosed herein can reduce the fasting glucose level by between 1% and 50%, between 1%, and 40%, between 1% and 30%, between 1% and 20%, between 1% and 10%, between 5% and 50%, between 5% and 40%, between 5% and 30%, between 5% and 20%, between 5% and 10%, between 10% and 50%, between 10% and 40%, between 10% and 30%, between 20% and 50%, between 20% and 40%, between 20% and 30%, between 30% and 50%, between 30% and 40%, or between 40% and 50%. In some cases, administering a composition as disclosed herein can reduce the fasting glucose level by between 1% and 80%, between 1% and 70%, between 1% and 60%, between 10% and 80%, between 10% and 70%, between 10% and 60%, between 20% and 80%, between 20% and 70%, between 20% and 60%, between 30% and 80%, between 30% and 70%, between 30% and 60%, between 40% and 80%, between 40% and 70%, between 40% and 60%, between 50% and 80%, between 50% and 70%, between 50% and 60%, between 60% and 80%, between 60% and 70%, or between 70% and 80%. In some cases, administering a composition disclosed herein can reduce the fasting glucose level by at least 1, 5, 10, 15, 20, 25, 30, 35, 40, 45, 50 mg/dL, or more

In some cases, administering a composition of the disclosure can reduce a fasting glucose level by at least 3 mg/dL, 5 mg/dL, 10 mg/dL, 15 mg/dL, 20 mg/dL, 25 mg/dL, 30 mg/dL, 35 mg/dL, 40 mg/dL, 45 mg/dL, 50 mg/dL, 60 mg/dL, 70 mg/dL, 80 mg/dL, 90 mg/dL, or 100 mg/dL, 150 mg/dL, 200 mg/dL, or more relative to pre-administration levels, or relative to a control subject that is administered a placebo and/or is not administered the same composition.

In some cases fasting glucose can be reduced by up to 20 mg/dL, 30 mg/dL, 40 mg/dL, 50 mg/dL, 60 mg/dL, 70 mg/dL, 80 mg/dL, 90 mg/dL, 100 mg/dL, 150 mg/dL, or 200 mg/dL, 250 mg/dL, 300 mg/dL, 350 mg/dL, 370 mg/dL, or 400 mg/dL relative to pre-administration levels, or relative to a control subject that is administered a placebo and/or is not administered the same composition. The change can be relative to pre-administration levels or relative to a healthy subject.

In some cases, the composition can be administered over a course of time, for example, days or weeks, and the fasting glucose levels can undergo reduction over that course of administration as determined by measuring the glucose levels at various time points during the length of treatment. For example, in some cases, the composition can be administered for at least 12 weeks. In some cases, after 6 months, the fasting glucose can be reduced by at least 5%, 10%, 20%, 30%, 40%, 50%, 60%, 70%, or 80% relative to pre-administration levels, or relative to a control subject that is administered a placebo and/or is not administered the same composition. In some cases, after 12 weeks, the fasting glucose can be reduced by at least 5%, 10%, 20%, 30%, 40%, 50%, 60%, 70%, or 80% relative to pre-administration levels, or relative to a control subject that is administered a placebo and/or is not administered the same composition. In some cases, after 10 weeks, the fasting glucose can be reduced by an average of at least 5%, 10%, 20%, 30%, 40%, 50%, 60%, 70%, or 80% relative to pre-administration levels, or relative to a control subject that is administered a placebo and/or is not administered the same composition. In some cases, after 8 weeks, the fasting glucose can be reduced by an average of at 5%, 10%, 20%, 30%, 40%, 50%, 60%, 70%, or 80% relative to pre-administration levels, or relative to a control subject that is administered a placebo and/or is not administered the same composition. In some cases, after 6 weeks, the fasting glucose can be reduced by an average of at least 5%, 10%, 20%, 30%, 40%, 50%, 60%, 70%, or 80% relative to pre-administration levels, or relative to a control subject that is administered a placebo and/or is not administered the same composition. In some cases, after 4 weeks, the fasting glucose can be reduced by an average of at least 5%, 10%, 20%, 30%, 40%, 50%, 60%, 70%, or 80% relative to pre-administration levels, or relative to a control subject that is administered a placebo and/or is not administered the same composition.

In some cases, administering a probiotic composition herein can reduce the fasting glucose in at least 5%10%, 20%, 30%, 40%, 50%, 60%, 70%, 80%, 90%, 95%, or 99% of subjects having prediabetes or type 2 diabetes.

In some cases, the fasting glucose level can be reduced from a diabetic level to a normal level. In some cases, the fasting glucose level can be reduced from a diabetic level to a pre-diabetic level. In some cases, the fasting glucose level can be reduced from a pre-diabetic level to a normal level. In some cases, the fasting glucose level can be reduced from a higher diabetic level to a lower diabetic level. In some cases, the fasting glucose level can be reduced from a higher pre-diabetic level to a lower pre-diabetic level.

In some cases, the reduction in fasting glucose levels over a defined time period may not exceed the reduction in fasting glucose levels over a shorter defined time period.

### Reduction of Postprandial Glucose Levels

Postprandial glucose levels can vary between subjects or in the same subject overtime. In some cases, postprandial glucose can be measured about 2 hours after a meal. In some cases, postprandial glucose can be measured between about 1.5 and 2.5 hours after a meal. In some subjects, a healthy postprandial glucose level can be less than 140 mg/dL. In some subjects, a postprandial glucose level indicative of prediabetes can be between 140 and 199 mg/dL. In some subjects, a postprandial glucose level indicative of diabetes can be 200 mg/dL or more.

Changes in postprandial glucose levels can occur in about 1 day, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 22 weeks, 24 weeks, or 26 weeks of beginning an intervention (e.g., after beginning to take a composition of the disclosure).

Changes in postprandial glucose levels can occur about 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 22 weeks, 24 weeks, or 26 weeks after beginning an intervention (e.g., after beginning to take a composition of the disclosure).

Postprandial glucose levels can be the amount of glucose measured in a blood sample taken a set period of time after food is consumed. Postprandial glucose can be measured after food is consumed following an overnight fast or after waking from sleep. Postprandial glucose can be measured about 0, 1, 2, or 3 hours after food is consumed.

Postprandial glucose can be measured multiple times in some subjects. The average, median, mode, highest, lowest, most recent level, or a combination thereof can be considered.

Postprandial glucose can be measured in a volume of blood which is at least 1.0, 1.5, 2.0, 2.5, 3.0, 3.5, 4.0, 4.5, or 5 or more μL of blood. The blood volume used to measure the glucose level can be at least 1 μL, 5 μL, 10 μL, 15 μL, 20 μL, 30 μL, 40 μL, 50 μL, 100 μL, 200 μL, 300 μL, 400 μL, 500 μL, 600 μL, 700 μL, 800 μL, 900 μL, 1 mL, 2 mL, 3 mL, 4 mL, 5 mL, 6 mL, 7 mL, 8 mL, 9 mL, or 10 mL of blood, or more.

In some cases, postprandial glucose can be measured without drawing blood samples, for example, using a continuous glucose monitoring device.

In some cases, administering a composition as disclosed herein can reduce the postprandial glucose level by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 60%, 70%, or 80% relative to pre-administration levels, or relative to a control subject that is administered a placebo and/or is not administered the same composition. In some cases, administering a composition as disclosed herein can reduce the postprandial glucose level by between 1% and 50%, between 1%, and 40%, between 1% and 30%, between 1% and 20%, between 1% and 10%, between 5% and 50%, between 5% and 40%, between 5% and 30%, between 5% and 20%, between 5% and 10%, between 10% and 50%, between 10% and 40%, between 10% and 30%, between 20% and 50%, between 20% and 40%, between 20% and 30%, between 30% and 50%, between 30% and 40%, or between 40% and 50% relative to pre-administration levels, or relative to a control subject that is administered a placebo and/or is not administered the same composition. In some cases, administering a composition as disclosed herein can reduce the postprandial glucose level by between 1% and 80%, between 1% and 70%, between 1% and 60%, between 10% and 80%, between 10% and 70%, between 10% and 60%, between 20% and 80%, between 20% and 70%, between 20% and 60%, between 30% and 80%, between 30% and 70%, between 30% and 60%, between 40% and 80%, between 40% and 70%, between 40% and 60%, between 50% and 80%, between 50% and 70%, between 50% and 60%, between 60% and 80%, between 60% and 70%, or between 70% and 80% relative to pre-administration levels, or relative to a control subject that is administered a placebo and/or is not administered the same composition. In some cases, administering a composition herein can reduce the postprandial glucose level by at least 1, 5, 10, 15, 20, 25, 30, 25, 40, 45, 50, 55, 60, 65. 70, 75, 80, 85, 90, 95, or 100 mg/dL, or more, relative to pre-administration levels, or relative to a control subject that is administered a placebo and/or is not administered the same composition.

In some cases, a postprandial glucose level is reduced by at least 3 mg/dL, 10 mg/dL, 20 mg/dL, 30 mg/dL, 40 mg/dL, 50 mg/dL, 60 mg/dL, 70 mg/dL, 80 mg/dL, 90 mg/dL, or 100 mg/dL. In some cases, postprandial glucose level can be reduced by as much as 370 mg/dL.

In some cases, the composition can be administered over a course of time, for example, days or weeks, and the postprandial glucose levels can undergo reduction over that course of administration as determined by measuring the postprandial glucose levels at various time points during the length of treatment. For example, in some cases, the composition can be administered for at least 12 weeks. In some cases, after 6 months, the postprandial glucose can be reduced by at least 2% relative to pre-administration levels, or relative to a control subject that is administered a placebo and/or is not administered the same composition. In some cases, after 12 weeks, the postprandial glucose can be reduced by at least 2% relative to pre-administration levels, or relative to a control subject that is administered a placebo and/or is not administered the same composition. In some cases, after 10 weeks, the postprandial glucose can be reduced by an average of at least 2% relative to pre-administration levels, or relative to a control subject that is administered a placebo and/or is not administered the same composition. In some cases, after 8 weeks, the postprandial glucose can be reduced by an average of at 2% relative to pre-administration levels, or relative to a control subject that is administered a placebo and/or is not administered the same composition. In some cases, after 6 weeks, the postprandial glucose can be reduced by an average of at least 2% relative to pre-administration levels, or relative to a control subject that is administered a placebo and/or is not administered the same composition. In some cases, after 4 weeks, the postprandial glucose can be reduced by an average of at least 2% relative to pre-administration levels, or relative to a control subject that is administered a placebo and/or is not administered the same composition.

In some cases, administering a probiotic composition herein can reduce the postprandial glucose in at least 5%, 10%, 20%, 30%, 40%, 50%, 60%, 70%, 80%, 90%, 95%, or 99% of subjects having prediabetes or type 2 diabetes.

In some cases, the postprandial glucose level can be reduced from a diabetic level to a normal level. In some cases, the postprandial glucose level can be reduced from a diabetic level to a pre-diabetic level. In some cases, the postprandial glucose level can be reduced from a pre-diabetic level to a normal level. In some cases, the postprandial glucose level can be reduced from a higher diabetic level to a lower diabetic level. In some cases, the postprandial glucose level can be reduced from a higher pre-diabetic level to a lower pre-diabetic level.

In some cases, the reduction in fasting glucose levels over a defined time period may not exceed the reduction in fasting glucose levels over a shorter defined time period.

### EXAMPLES

### Example 1: Effect of Compositions on Outcomes

A balanced, parallel-arm, double-blind, placebo-controlled study was conducted. 60 subjects having early stage type 2 diabetes were recruited and divided into 3 groups: placebo, Formulation 1 (containing butyrate-producing microbes), and Formulation 2 (containing at least one butyrate-producing microbe and at least one mucin-regulating microbe). Subjects in the placebo group received a placebo treatment with no microbes. Subjects in the Formulation 1 group received a probiotic composition with 3 microbes, comprising butyrate-producing microbes. Subjects in the Formulation 2 group received a probiotic composition with 5 microbes, comprising mucin-regulating and butyrate-producing microbes. The placebo, Formulation 1, and Formulation 2 were administered in acid-resistant, plant-based capsules comprising water, hydroxypropyl methylcellulose, hydroxypropyl methylcellulose phthalate, and propylene glycol.

Formulation 1 included the following microbes: Clostridium beijerinckii, Clostridium butyricum, and Bifidobacteria infantis. Formulation 2 included the following microbes: Clostridium beijerinckii, Clostridium butyricum, Bifidobacterium infantis, Akkermansia muciniphila, and Eubacterium halli. Subjects in the Formulation 2 group were administered the microbes in the amounts shown in Table 3:

TABLE 3
Specifications of Formulation 2

| Microbe/component | CFU per day | mL per day | mg per day |
|---|---|---|---|
| Clostridium beijerinckii | 1.15 × 10\hat{ }9 | 0.232 | 45.8 |
| Clostridium butyricum | 3.34 × 10\hat{ }8 | 0.016 | 4.8 |
| Eubacterium hallii | 9.00 × 10\hat{ }8 | 0.071 | 37.2 |
| Akkermansia muciniphila | 1.16 × 10\hat{ }9 | 0.012 | 8.0 |
| Bifidobacterium infantis | 2.00 × 10\hat{ }8 | 0.003 | 2.0 |
| Inulin | — | — | 276.0 |
| Colloidal Silicone dioxide | — | — | 108.0 |

Subjects were administered the composition twice per day over a course of 12 weeks. At the start of the study (day 0) and at the end of the 12 weeks, fasting blood glucose, hA1C, and glucose area under the curve (AUC) after a meal tolerance test were measured.

Subjects that were administered Formulation 1 or Formulation 2 did not exhibit significant or intolerable adverse effects.

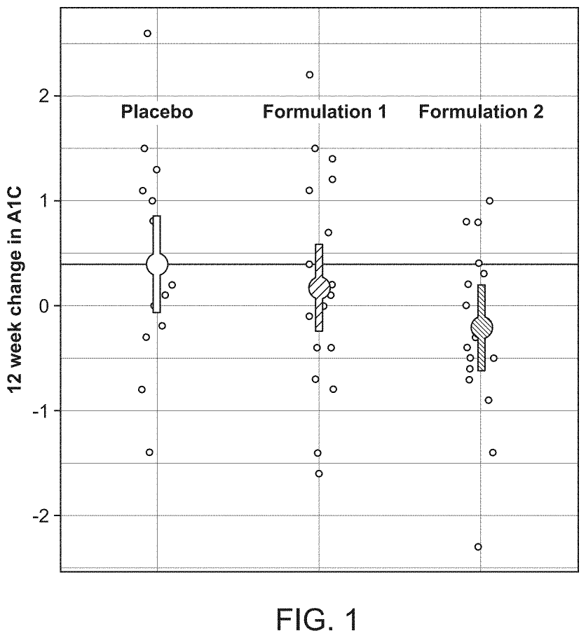

At the start of the study (day 0) and after 12 weeks, hA1C was measured in blood samples from subjects from all three groups. The change in hA1C between day 0 and week 12 was calculated for each subject, and data were compared to the placebo group. Results are shown in FIG. 1. The Formulation 1 group displayed a reduction in hA1C levels compared to the placebo group. The Formulation 2 group displayed a significant (p=0.05) reduction in hA1C compared to the placebo group by an average of 0.6. The Formulation 2 group also displayed a reduction in hA1C from day 0 to week 12. Thus, Formulation 2, comprising both butryrate-producing and mucin-regulating microbes, lowered subject hA1C levels over the 12 week period and when compared with the placebo group.

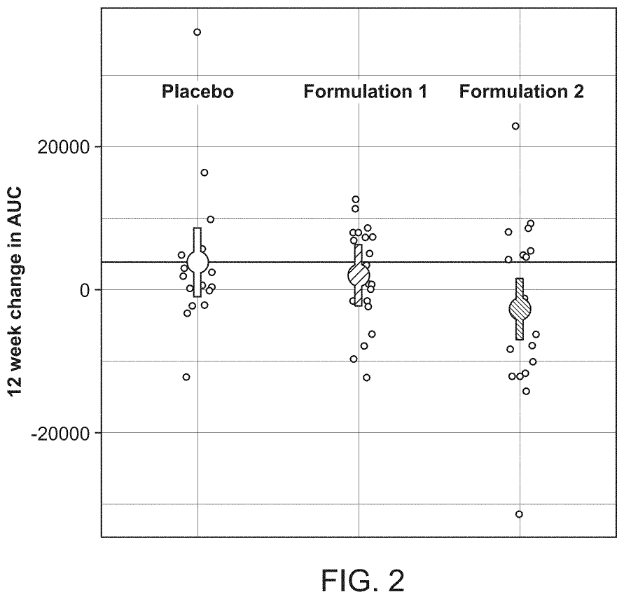

At the start of the study (day 0) and at the end of the 12 weeks, a meal tolerance test was administered, blood glucose was measured serially over a period of three hours, and the area under the curve of glucose vs. time was calculated for subjects from all three groups. Briefly, subjects were administered a meal, and glucose concentration in the blood was measured over a period of 3 hours at 0, 30, 60, 90, 120, and 180 minutes after the meal (meal tolerance test, or MTT). The area under the curve (AUC) of glucose vs. time was calculated for each subject. The change in AUC between day 0 and week 12 was calculated for each subject, and data were compared to the placebo group. Results are shown in FIG. 2. The Formulation 1 group displayed a slight reduction in glucose AUC after MTT compared to the placebo group. The Formulation 2 group displayed a significant (p=0.05) reduction in glucose AUC after MTT compared to the placebo group, by an average of 15.1%. The Formulation 2 group also displayed a reduction in AUC from day 0 to week 12. Thus, the 5 microbe probiotic composition comprising butyrate-producing and mucin-regulating microbes lowered subject AUC after MTT levels over the 12 week period and when compared with the placebo.

After 12 weeks, fasting glucose levels were measured from blood samples from subjects from all three groups. No significant differences in fasting blood glucose levels were found between the placebo group and the Formulation 1 group or Formulation 2 group.

### Example 2: Effect of Compositions Co-Administered with Metformin

Subjects were administered the placebo, Formulation 1, or Formulation 2 over a course of 12 weeks as in example 1. Subjects were also administered metformin over the same 12 weeks. At the start of the study (day 0) and at the end of the 12 weeks, fasting blood glucose, hA1C, and glucose area under the curve (AUC) after a meal tolerance test were measured as in example 1. Subjects that were administered Formulation 1 or Formulation 2 did not exhibit significant or intolerable adverse effects.

Subjects receiving Formulation 2 and metformin exhibited a decrease in hA1C compared to subjects receiving the placebo and metformin. The Formulation 2 plus metformin group also displayed a reduction in hA1C from day 0 to week 12.

Subjects receiving Formulation 2 and metformin displayed a reduction in the glucose AUC after MTT compared to the group receiving the placebo plus metformin. The Formulation 2 plus metformin group also displayed a reduction in AUC from day 0 to week 12.

No significant differences in fasting blood glucose levels were found between the placebo group and the Formulation 1 group or Formulation 2 group.

Thus, Formulation 2, comprising both butyrate-producing and mucin-regulating microbes, lowered hA1C and AUC after MTT levels in subjects receiving metformin over the 12 week period, and when compared to subjects receiving a placebo plus metformin.

### Example 3: Comparison to Marketed Drugs

Formulation 2, containing a mucin-regulating microbe and one or more butyrate-producing microbes, provided comparable therapeutic effects relative to other marketed drugs, without safety issues or inducing hypoglycemia when administered to subjects suffering from early stage type-II diabetes. Table 4 provides a comparison of Formulation 2 to drugs marketed for the treatment of diabetes.

TABLE 4
Comparison of Formulation 2 to Drugs Marketed for the
Treatment of Diabetes

| Drug | A1C Reduction | Hypoglycemia | Safety Issues |
|---|---|---|---|
| Formulation 2 | −0.6% | None | None known |
| Sulfonylureas | −0.7% | ++ | Increased |
| | | | cardiovascular |
| | | | (CV) risk |
| | | | Weight gain |
| Thiazolidinediones | −1.0% | + | Edema |
| | | | Bone fractures |
| | | | (CV Risk) |
| Gliptins | −0.7% | None | ?Pancreatitis |
| GLP-1 Agonists | | | |
| Daily | | | |
| Weekly | −0.8-1.2% | None | ?Pancreatitis |
| Oral | −1.2-1.6% | | |
| SGLT2 Inhibitors | | + | Dehydration |
| | | | Amputations |
| Basal Insulin | Highly Variable | ++++ | Hypoglycemia |
| | (−0.7-1.0%) | | Weight gain |

### Example 4: Effect of Compositions Co-Administered with Sulfonylurea

Subjects having early stage type 2 diabetes were recruited and divided into 3 groups: placebo, Formulation 1 (containing butyrate-producing microbes), and Formulation 2 (containing at least one butyrate-producing microbe and at least one mucin-regulating microbe). Subjects in the placebo group received a placebo treatment with no microbes. Subjects in the Formulation 1 group received a probiotic composition with 3microbes, comprising butyrate-producing microbes. Subjects in the Formulation 2group received a probiotic composition with 5microbes, comprising mucin-regulating and butyrate-producing microbes. Formulation 1included the following microbes: Clostridium beijerinckii, Clostridium buyricum, and Bifidobacterium infantis. Formulation 2included the following microbes: Clostridium beierinckii, Clostridium butyricum, Bifidobacterium infantis, Akkermansia muciniphila, and Eubacterium hallii.

Subjects were administered the placebo or the composition (either Formulation 1 or Formulation 2) twice per day over the course of 12 weeks. A subset of subjects in each group continued a pre-established regimen of sulfonylurea over the same 12 weeks. At the start of the study (day 0) and at the end of the 12 weeks, hA1C was measured. Additionally, at the start of the study and at the end of the 12 weeks, a meal tolerance test was administered, blood glucose was measured serially over the period of three hours, and the area under the curve of glucose vs. time was calculated.

Subjects that were administered Formulation 1 or Formulation 2 did not exhibit significant or intolerable adverse effects.

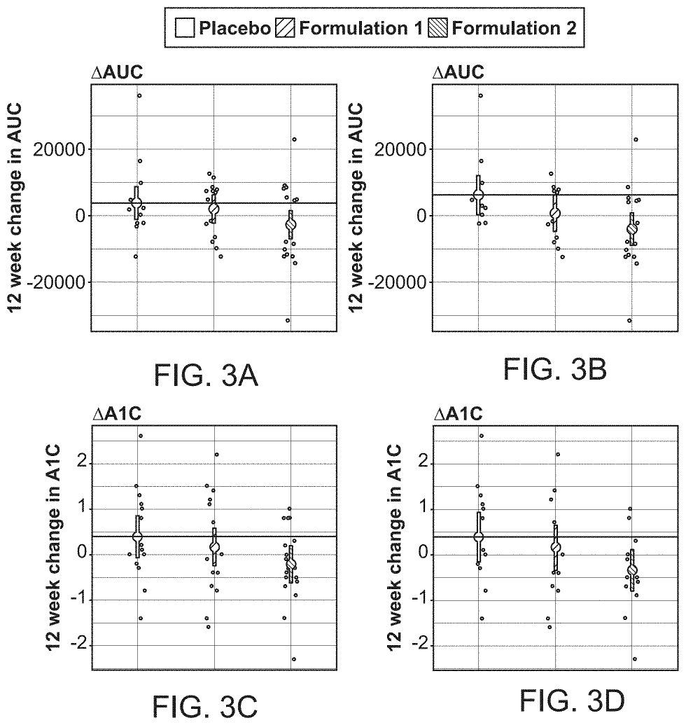

At the start of the study and after 12 weeks, the glucose area under the curve was determined for subjects from all three groups. Briefly, subjects were administered a meal, and glucose concentration in the blood was measured over a period of 3 hours at 0, 30, 60, 90, 120, and 180 minutes after the meal (a meal tolerance test, or MTT). The area under the curve (AUC) of glucose vs. time was calculated for each subject. The change in AUC between day 0 and week 12 was calculated for each subject. When all subjects were considered (regardless of sulfonylurea administration), the Formulation 1 group displayed a slight reduction in glucose AUC after MTT compared to the placebo group, and the formulation 2 group displayed a significant (p=0.05) reduction in glucose AUC after MTT compared to the placebo group, by an average of 15.1% (FIG. 3A). When patients receiving sulfonylurea were excluded from the analysis, the change in AUC after MTT increased in magnitude to −24.4%, as depicted in FIG. 3B. These results suggest that in some cases, compositions and methods of the disclosure can exhibit increased efficacy for reducing glucose AUC in subjects not receiving sulfonylurea compared to subjects receiving sulfonylurea.

At the start of the study and after 12 weeks, hemoglobin A1C (hA1C) was measured in blood samples from subjects from all three groups. When all subjects were considered (regardless of sulfonylurea administration), the Formulation 1 group displayed a reduction in hA1C levels compared to the placebo group. The Formulation 2 group displayed a significant (p=0.05) reduction in hA1C compared to the placebo group by an average of 0.6 (FIG. 3C).

When patients receiving sulfonylurea were excluded from the analysis, the change in hemoglobin A1C increased in magnitude to −0.74, as depicted in FIG. 3D. These results suggest that in some cases, compositions and methods of the disclosure can exhibit increased efficacy for reducing hA1C in subjects not receiving sulfonylurea compared to subjects receiving sulfonylurea.

Thus, in some cases, a lower dose or no dose of sulfonylurea can promote a therapeutic outcome for the administered composition.

### Example 5: Effect of Compositions on Blood Glucose Control

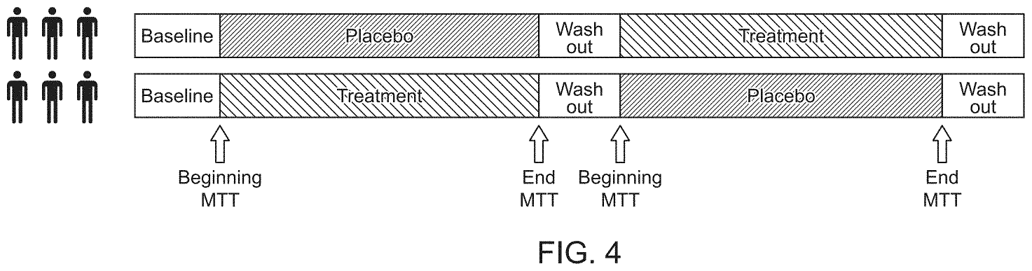

Six subjects were enrolled in a placebo-controlled, double-blinded, randomized crossover trial. Three subjects were designated as prediabetic, the other three subjects were designated as healthy. The subjects were randomly distributed into two groups of three subjects. During the study, each group went through a series of phases as illustrated in FIG. 4. After a baseline period of three days, one group began a two week treatment phase and the other group began a two week placebo phase. During the placebo phase, subjects were administered a colloidal silicon dioxide placebo twice per day. During the treatment phase, subjects were administered a composition twice per day of isolated and purified microbes that contained a prebiotic, a mucin-regulating microbe, and at least one butyrate-producing microbe. Subjects took 3 pills in the morning and 3 pills in the evening. The composition of isolated and purified microbes contained Clostridium beijerinckii, Clostridium butyricum, Bifidobacterium infantis, Akkermansia muciniphila, and Eubacterium halli. After the two week treatment or placebo phase, both groups went through a three day “washout” phase, with no placebo or treatment composition administered. Following the washout phase, the placebo/treatments were “crossed over”: the group that had previously undergone a treatment phase began a placebo phase, and the group that had previously undergone a placebo phase began a treatment phase.

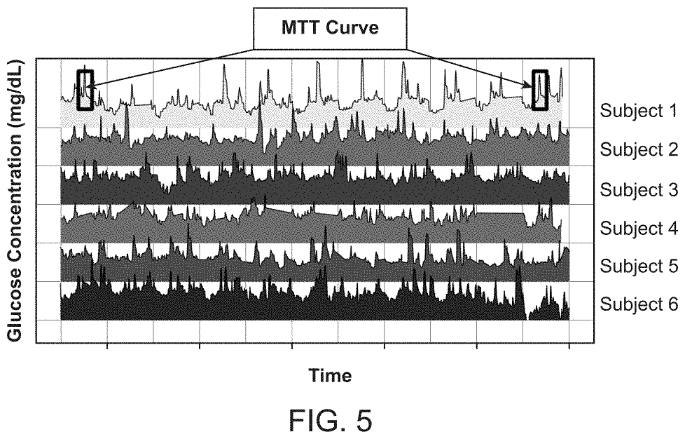

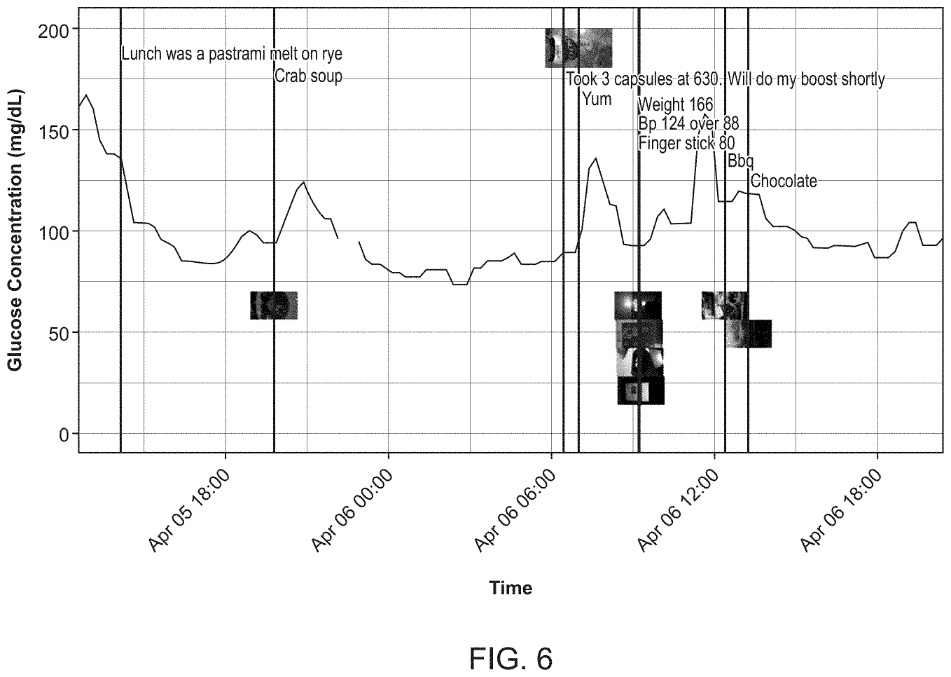

Throughout the study, glucose monitors were used to measure glucose levels in each subject using continuous glucose monitoring (CGM). An example of data obtained from CGM is provided in FIG. 5. Subjects logged their food, drink, and activity using a mobile phone application. An example of a subject logging their food, drink, and activity is illustrated in FIG. 6.

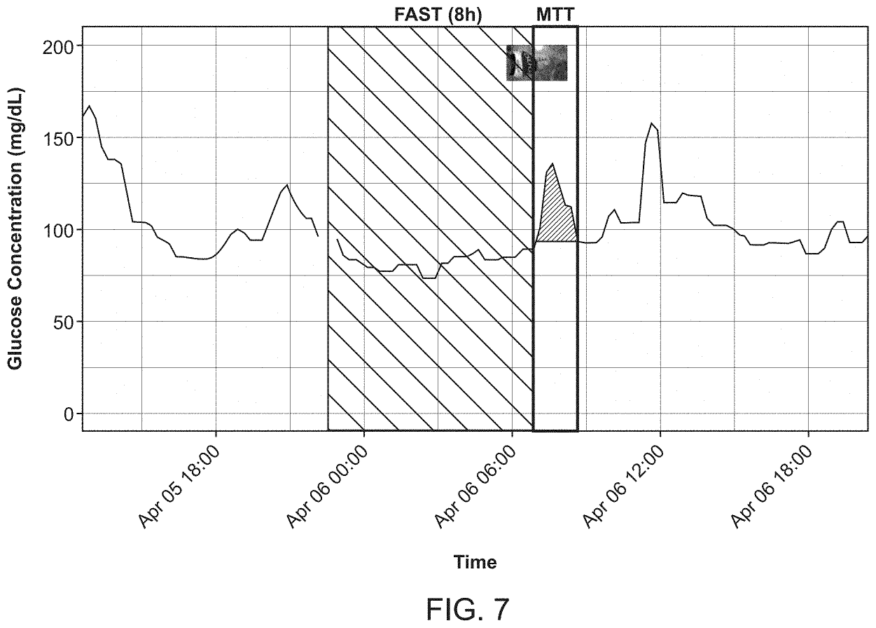

At the beginning and end of each treatment and placebo phase, subjects underwent a fasted meal tolerance test (MTT) using CGM to measure interstitial glucose (a proxy for blood glucose). A standardized meal was consumed after an overnight fast, and participants were asked to fast for one additional hour after the meal, so as not to interfere with the MTT measurements. FIG. 7 provides an example of data from a subject logging their MTT. Subjects logged the start of the MTT with by taking a picture of the standardized meal in the mobile phone application. The shaded area represents an overnight fast preceding the MTT. The boxed area represents the glucose spike from the MTT.

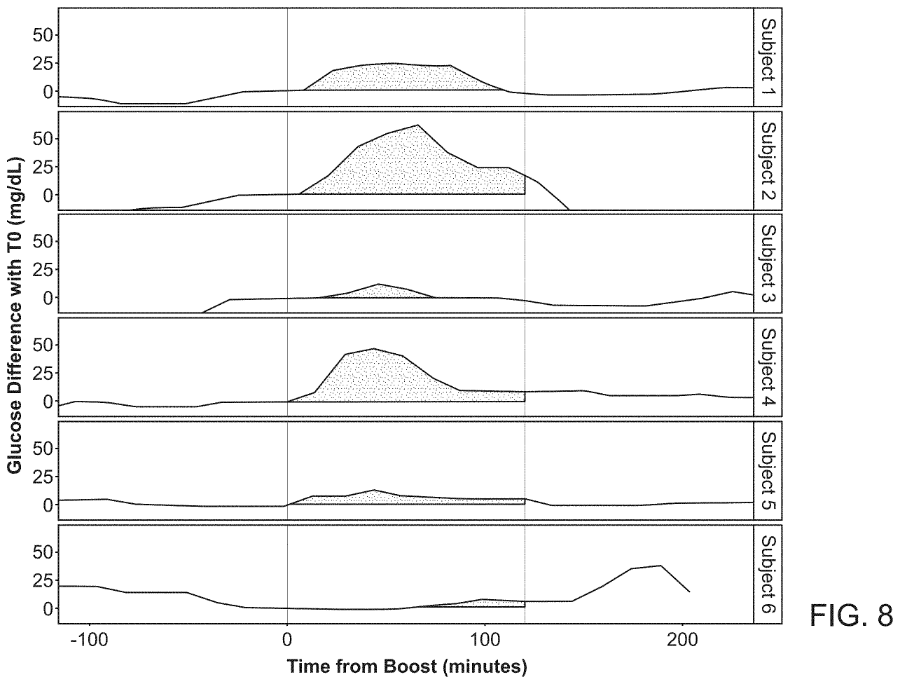

FIG. 8 provides glucose concentration curves for the six subjects undergoing a MTT at baseline (before the treatment phase, i.e., before receiving the composition of isolated and purified microbes or placebo). Subjects 1, 2, and 4 exhibited significant spikes in blood glucose after consuming the standardized meal, consistent with their pre-study designation as prediabetic. Subjects 3 and 5 exhibited small increases in blood glucose consistent with their pre-study designation as healthy. Subject 6 did not exhibit a plausible glucose response, and was excluded from further analysis prior to unblinding.

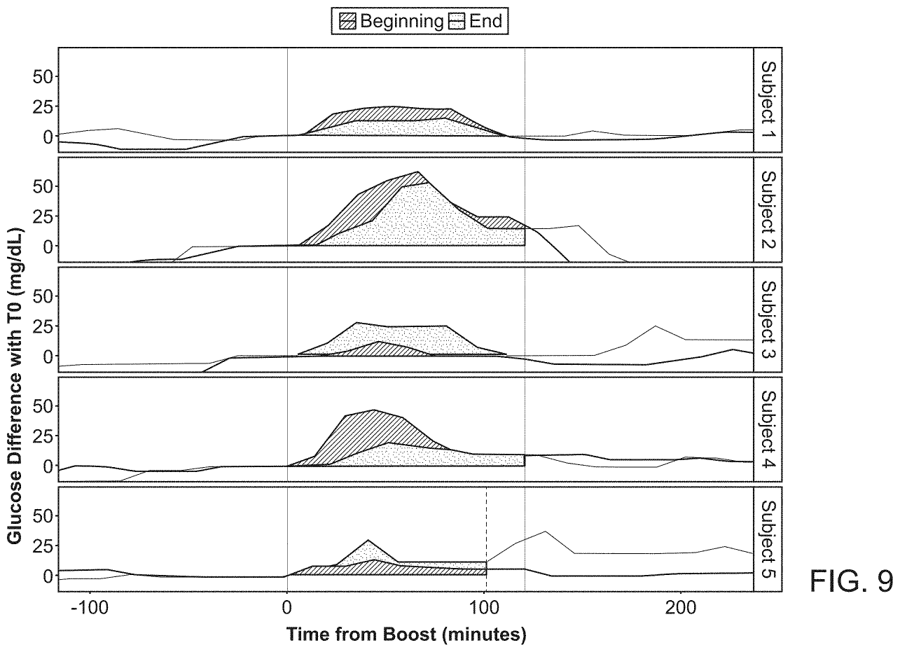

FIG. 9 provides glucose concentration curves for the five remaining subjects undergoing a MTT at the end of the treatment phase (i.e., after receiving the composition of isolated and purified microbes or placebo), superimposed on the glucose concentration curves from the beginning of the treatment phase. The area under the glucose concentration curves is lower for subjects 1, 2, and 4 after the treatment phase, indicating that the composition comprising isolated and purified microbes can reduce AUC in prediabetic subjects.

Glucose area under the concentration curves (AUC) were calculated for each subject using the CGM data. For each subject, the AUC at the beginning of the placebo/treatment phase was compared to the AUC at the end of the placebo/treatment phase, and the change in AUC between beginning and end of the phase calculated using the formula:

ΔAUC=AUC_{END}−AUC_{BEGINNING}.

A negative ΔAUC value indicates an improvement in the control of blood glucose concentration, as AUC is lower at the end of the phase than at the beginning of the phase.

The cross-over design of the trial allowed comparison of ΔAUC between the placebo phase and the treatment phase for each subject. The difference in ΔAUC between the phases was calculated using the formula:

ΔΔAUC=ΔAUC_{TREATMENT}−ΔAUC_{PLACEBO}.

A negative ΔΔAUC value indicates that treatment resulted in improved blood glucose control compared to placebo.

The ΔAUC values for each phase and the ΔΔAUC values for each subject are provided in Table 5.

TABLE 5
ΔAUC values for each phase and the ΔΔAUC values for each
subject administered a composition of the disclosure

| | | | Placebo % | | Treatment | |
|---|---|---|---|---|---|---|
| | Pre-study | Placebo | change | Treatment | % change | |
| Subject | designation | ΔAUC | AUC | ΔAUC | AUC | ΔΔAUC |
| 1 | Prediabetic | 74.3 | 5.91 | −759 | −42.47 | −833.3 |
| 2 | Prediabetic | 1512.7 | 45.82 | −1133.7 | −28.8 | −2646.4 |
| 3 | Healthy | 704.9 | 51.17 | 1352.3 | 380.5 | 647.4 |
| 4 | Prediabetic | 452.5 | 26.45 | −1499.4 | −55.67 | −1952 |
| 5 | Healthy | −234.2 | −22 | 429.2 | 56.22 | 663.4 |

For subjects 1, 2, and 4, but not subjects 3, and 5, AUC decreased between the start of the treatment phase and the end of the treatment phase, indicating the composition comprising isolated and purified microbes can improve control of blood glucose in subjects with prediabetes. ΔΔAUC also decreased in subjects 1, 2, and 4, indicating that the composition comprising isolated and purified microbes exhibits a superior ability to improve control of blood glucose compared to placebo.

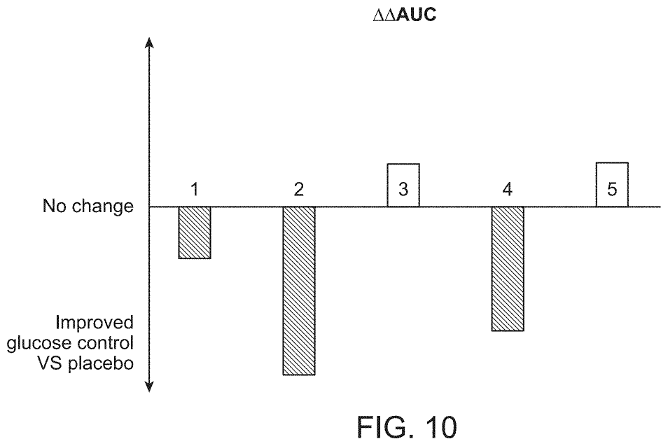

FIG. 10 illustrates the AA-AUC for subjects 1-5.

### Example 6: Short-Chain Fatty Acid Production

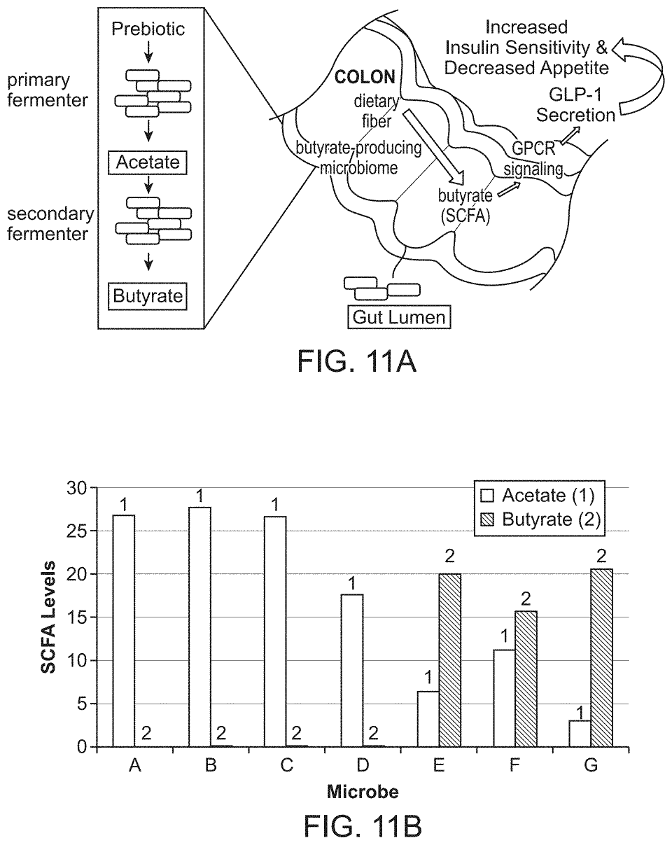

FIG. 11A illustrates a strategy to alter short chain fatty acid (SCFA) metabolism in a subject. Microbes in the colon can convert dietary fiber into butyrate, which can have beneficial downstream effects, for example, by altering G-protein coupled receptor (GPCR) signaling, altering GLP-1 secretion, increasing insulin sensitivity, decreasing appetite, or a combination thereof. Compositions and methods of the disclosure can be used to alter a microbiome in a subject to promote butyrate production. For example, a microbiome in a subject can be modified to comprise increased levels of one or more primary fermenter microbes that can convert a prebiotic into a butyrate intermediate (e.g., an intermediate that can serve as a substrate for butyrate production, such as acetate), and to comprise increased levels of one or more secondary fermenter microbes that can convert the butyrate intermediate into butyrate.

FIG. 11B illustrates short chain fatty acid levels produced by microbes of the disclosure. Microbes A-D primarily produced acetate, which can be a butyrate intermediate (e.g., serve as a substrate for butyrate production by a butyrate-producing microbe). Microbes E, F, and G primarily produced butyrate. A combination of a first microbe producing a butyrate intermediate (e.g., any of microbes A-D) and a second microbe converting the intermediate to butyrate (e.g., any of microbes E-G) can be utilized for treating a condition. In one non-limiting example, strain A can be Bifidobacterium adolescentis (BADO). In one non-limiting example, strain B can be Bifidobacterium infantis (BINF). In one non-limiting example, strain C can be Bifidobacterium longum (BLON). In one non-limiting example, strain D can be Clostridium indolis (CIND). In one non-limiting example, strain E can be Clostridium beijerinckii (CBEI). In one non-limiting example, strain F can be Clostridium butyricum (CBUT). In one non-limiting example, strain G can be Eubacterium hallii (EHAL).

### Example 7: Safety Study in an Animal Model

A 28 day safety study was conducted in Sprague Dawley Rats. Rats were orally administered a placebo or a composition of the disclosure for 28 days. A vial of lyophilized microbes (comprising Clostridium beijerinckii, Clostridium butyricum, Bifidobacterium infantis, Akkermansia muciniphila, and Eubacterium hallii) was reconstituted in diluent packaged under anaerobic conditions. The liquid was drawn into a syringe, and an appropriate amount was orally administered to each animal. Clinical observations were performed routinely at each scheduled time point. Body weights were recorded prior to each administration of composition or placebo. Animals were sacrificed on day 28 or day 35 (after a 7 day washout period), and necropsies were conducted. Blood (as much as possible) was collected from all animals. An aliquot of the blood was processed appropriately and analyzed for clinical chemistry and complete blood count (CBC) with differential. Data from clinical observation of the animals, plasma chemistry, hematology panels, and necropsy evaluation confirmed that there were no adverse effects on the rodents from the administered compositions. No differences in health indicators were observed between the rats receiving the placebo and the rats receiving the composition.

### Example 8: Efficacy Study in an Animal Model

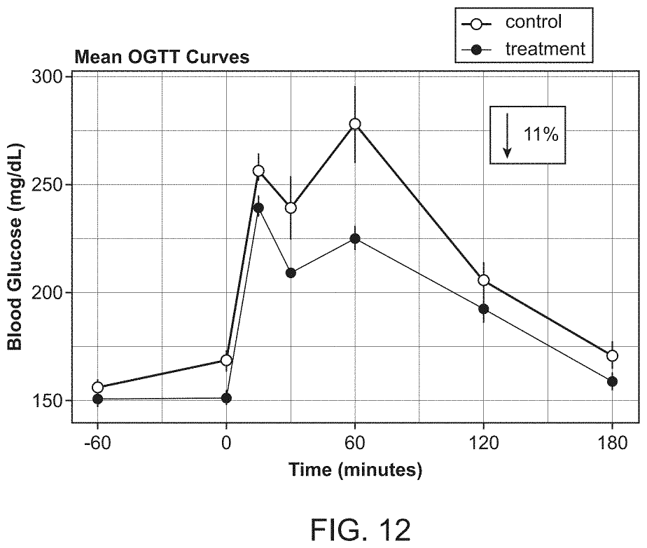

A preclinical study was conducted in a diet-induced obesity mouse model. 45 five-week-old C57BL/6 mice were given ad libitum access to a 60% high fat diet (Research Diet, #D12492) for 32 weeks to induce obesity. After 32 weeks on the diet, the mice were administered a composition of the disclosure once daily by oral gavage (“treatment”). The composition comprised a mucin-regulating microbe, and a butyrate-producing microbe. The composition comprised a primary fermenter and a secondary fermenter. In one non-limiting example, the composition comprised a prebiotic (e.g. inulin), and isolated and purified microbes of the strains Clostridium butyricum, Clostridium beijerinckii, Bifidobacterium longum, Bifidobacterium infantis, Bifidobacterium adolescentis, Akkermansia muciniphila, Eubacterium hallii, and Clostridium indolis. FIG. 12 depicts an example data set from an oral glucose tolerance test (OGTT) performed 14 days after beginning treatment. Mice administered the composition exhibited significantly lower blood glucose levels during the OGTT than control mice.

### Example 9: Detection of Microbes in Human Stool Via qPCR

Pills comprising microbes of the disclosure were manufactured in compliance with current Good Manufacturing Practice (cGMP) regulations. Coated capsules comprising the substantially dry population of lyophilized microbes were used. The capsules were designed to maximize survival time in stomach acid to allow delivery to the intestine. The capsules were stored refrigerated except for dispensing to the subject and at the time of consumption.

Human subjects were orally administered pills comprising low or high doses of the microbes at dosages according to Table 6. Subjects consumed the capsules within 30 minutes prior to the start of breakfast and dinner for 14 days. A lower dose was administered from day 0-6, and the dose was increased 5-fold for days 7 to 14. Subjects then entered a 14-day wash-out period when no study food product is administered.

TABLE 6
Dosage

| | Clostridium | Clostridium | Bifodobacterium |
|---|---|---|---|
| | beijerinckii, | butyricum | infantis |
| Study Days | (CFU/dose) | (CFU/dose) | (CFU/dose) |
| Dose 1/Low Dose | 7.0 × 10⁹ | 4.0 × 10⁹ | 2.0 × 10⁸ |
| (Days 0-6) | | | |
| Dose 2/High Dose | 3.5 × 10¹⁰ | 2.0 × 10¹⁰ | 1.0 × 10⁹ |
| (Days 7-14) | | | |

Stool samples were collected from the subjects before they commenced taking the pills, while they were taking the pills (day 7—low dose; and day 14—high dose), and after a 14 day washout period in which they ceased taking the pills.

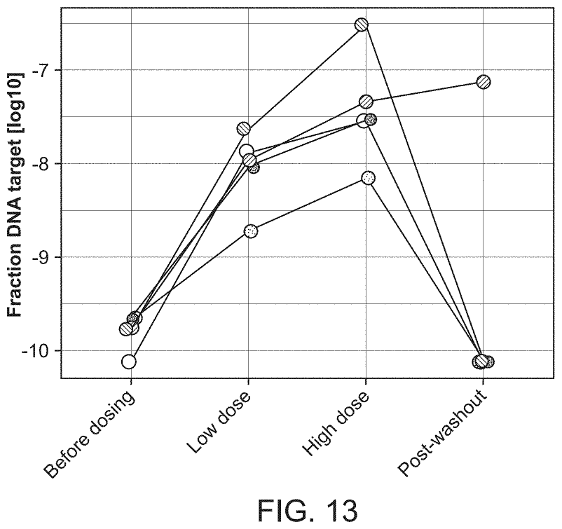

Samples were subjected to nucleic acid extraction and quantitative real time PCR (qPCR) to detect the microbes of the disclosure. The abundance of the microbes in stool samples increased after subjects began taking the pills (FIG. 13). Higher levels of the microbes were detected in subjects taking the higher dose pills. After the washout period, the level of microbes detected in the stool samples decreased for most subjects, but persisted in one subject, indicating possible engraftment of the microbe in that subject.

### Example 10: Example Composition to Manage Blood Sugar and Type 2 Diabetes

A composition of the disclosure is used to manage blood sugar and type 2 diabetes.

The composition is for the dietary management of type 2 diabetes.

The composition is a composition of medical probiotics.

The composition manages healthy A1C and blood glucose levels.

The composition is provided in capsule form (for example, as a package of 60 capsules).

The composition is vegan. The composition is non-genetically modified (non-GMO).

The composition is perishable and is to be kept refrigerated.

The composition is best if used within 2 months of opening.

The composition is used only under medical supervision.

The composition is taken daily with food, as 1 pill in the morning and 1 pill in the evening.

The composition's precise strains of probiotics and prebiotics restore the body's natural ability to metabolize fiber and regulate blood sugar.

The composition results in a statistically significant reduction in HbA1C and blood sugar spikes in people with type 2 diabetes in a randomized, double-blinded, placebo-controlled clinical trial across multiple sites in the United States.

The composition comprises as ingredients: Probiotic Blend (Clostridium beijerinckii WB-STR-0005, Clostridium butyricum WB-STR-0006, Bifidobacterium infantis 100, Akkermansia muciniphila WB-STR-0001, Eubacterium hallii WB-STR-0008), Chicory Inulin and Oligofructose, Fruit and Vegetable Juice (Color), Magnesium Stearate, Capsule (Water, Hydroxylpropyl Methylcellulose Phthalate, Hydroxypropyl Methylcellulose).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-300. (canceled)

301. A method of reducing hemoglobin A1C (hA1C) levels in a subject, comprising administering to the subject an oral composition comprising Clostridium beijerinckii, Akkermansia muciniphila, and Eubacterium hallii, wherein hemoglobin A1C (hA1C) levels are reduced in comparison to hA1C levels in the subject before administration of the oral composition to the subject.

302. The method of claim 301, wherein the reducing hemoglobin A1C (hA1C) levels in a subject refers to reducing hA1C levels by at least 0.2% of total hemoglobin in the subject in comparison to hA1C levels in the subject before administration of the oral composition to the subject.

303. The method of claim 301, wherein the oral composition further comprises at least one microbe comprising an rRNA sequence with at least 97% sequence identity to an rRNA of a microbe selected from the group consisting of Anaerostipes caccae, Bacteroides stercoris, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium butyricum, Clostridium colinum, Clostridium coccoides, Clostridium indolis, Clostridium nexile, Clostridium orbiscindens, Clostridium propionicum, Clostridium xylanolyticum, Collinsella aerofaciens, Enterococcus faecium, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus faecis, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburiafaeccis, Roseburia hominis, Roseburia intestinalis, Lacatobacillus bifidus, Lactobacillus johnsonii, Lactobacilli, Acidaminococcus fermentans, Acidaminococcus intestine, Blautia hydrogenotrophica, Citrobacter amalonaticus, Citrobacter freundii, Clostridium aminobutyricum Clostridium bartlettii, Clostridium cochlearium, Clostridium kluyveri, Clostridium limosum, Clostridium malenominatum, Clostridium pasteurianum, Clostridium peptidivorans, Clostridium saccharobutylicum, Clostridium sporosphaeroides, Clostridium sticklandii, Clostridium subterminale, Clostridium symbiosum, Clostridium tetanomorphum, Eubacterium oxidoreducens, Eubacterium pyruvativorans, Methanobrevibacter smithii, Morganella morganii, Peptoniphilus asaccharolyticus, and any combination thereof.

304. The method of claim 301, further comprising administering metformin to the subject and wherein the hA1C levels in the subject is reduced by at least 0.6% of total hemoglobin, in comparison to administration of metformin alone.

305. The method of claim 301, wherein glucose AUC is reduced by at least 10% in comparison to glucose AUC levels in the subject before administration of the oral composition to the subject.

306. The method of claim 301, wherein the subject is a human.

307. The method of claim 301, wherein the oral composition is in a unit dosage form.

308. The method of claim 301, wherein the oral composition further comprises at least an enteric coating or at least one preservative.

309. The method of claim 301, wherein the oral composition comprises at least 1×10⁶ CFU of each of the Clostridium beijerinckii, Akkermansia muciniphila, and Eubacterium hallii.

310. The method of claim 301, wherein insulin sensitivity is increased in the subject, in comparison to insulin sensitivity in the subject before administration of the oral composition to the subject.

311. The method of claim 301, wherein blood glucose levels are stabilized in the subject.

312. A method of reducing hemoglobin A1C (hA1C) levels in a subject, by administering to the subject an oral composition comprising:
a microbial composition comprising Clostridium beijerinckii, Akkermansia muciniphila, and Eubacterium hallii, and
metformin;
wherein, the subject has a disorder selected from the group consisting of Type II diabetes, insulin insensitivity and insulin resistance, wherein hA1C levels are reduced in comparison to hA1C levels in the subject before administration of the composition to the subject.

313. The method of claim 312, wherein the reducing hA1C levels lowers A1C levels by at least 0.2% of total hemoglobin in the subject.

314. The method of claim 312, wherein the composition further comprises at least one microbe comprising an rRNA sequence with at least 97% sequence identity to an rRNA of a microbe selected from the group consisting of Anaerostipes caccae, Bacteroides stercoris, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium butyricum, Clostridium colinum, Clostridium coccoides, Clostridium indolis, Clostridium nexile, Clostridium orbiscindens, Clostridium propionicum, Clostridium xylanolyticum, Collinsella aerofaciens, Enterococcus faecium, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus faecis, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburiafaeccis, Roseburia hominis, Roseburia intestinalis, Lacatobacillus bifidus, Lactobacillus johnsonii, Lactobacilli, Acidaminococcus fermentans, Acidaminococcus intestine, Blautia hydrogenotrophica, Citrobacter amalonaticus, Citrobacter freundii, Clostridium aminobutyricum Clostridium bartlettii, Clostridium cochlearium, Clostridium kluyveri, Clostridium limosum, Clostridium malenominatum, Clostridium pasteurianum, Clostridium peptidivorans, Clostridium saccharobutylicum, Clostridium sporosphaeroides, Clostridium sticklandii, Clostridium subterminale, Clostridium symbiosum, Clostridium tetanomorphum, Eubacterium oxidoreducens, Eubacterium pyruvativorans, Methanobrevibacter smithii, Morganella morganii, Peptoniphilus asaccharolyticus, and any combination thereof.

315. The method of claim 312, wherein the subject is a human.

316. The method of claim 312, wherein the composition is in a unit dosage form.

317. The method of claim 312, wherein the composition further comprises at least an enteric coating or at least one preservative.

318. The method of claim 312, wherein the composition comprises at least 1×10⁶ CFU of each of the microbes in the composition.


SEQUENCE LISTING (6 sequences)

SEQ ID NO: 1 - DNA, 1523 bp, akkermansia muciniphila
aaaattaatttgatggagagtttgattctggctcagaacgaacgctggcggcgtggataagacatgcaagtcgaacgagagaattgctagcttgctaataattctctagtggcgcacgggtgagtaacacgtgagtaacctgcccccgagagcgggatagccctgggaaactgggattaataccgcatagtatcgaaagattaaagcagcaatgcgcttggggatgggctcgcggcctattagttagttggtgaggtaacggctcaccaaggcgatgacgggtagccggtctgagaggatgtccggccacactggaactgagacacggtccagacacctacgggtggcagcagtcgagaatcattcacaatgggggaaaccctgatggtgtgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagggattagatacccctgtagtcctggcagtaaacggtgcacgcttggtgtgcggggaatcgaccccctgcgtgccggagctaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtaatgaacaacatgtgaaagcatgcgactcttcggaggcgttacacaggtgctgcatggccgtcgtcagctcgtgtcgtgagatgtttggttaagtccagcaacgagcgcaacccctgttgccagttaccagcacgtgaaggtggggactctggcgagactgcccagatcaactgggaggaaggtggggacgacgtcaggtcagtatggcccttatgcccagggctgcacacgtactacaatgcccagtacagagggggccgaagccgcgaggcggaggaaatcctgaaaactgggcccagttcggactgtaggctgcaacccgcctacacgaagccggaatcgctagtaatggcgcatcagctacggcgccgtgaatacgttcccgggtcttgtacacaccgcccgtcacatcatggaagccggtcgcacccgaagtatctgaagccaaccgcaaggaggcagggtcctaaggtgagactggtaactgggatgaagtcgtaacaaggtagccgtaggggaacctgcggctggatcacctcctttct

SEQ ID NO: 2 - DNA, 1507 bp, akkermansia muciniphila
agagtttgattctggctcagaacgaacgctggcggcgtggataagacatgcaagtcgaacgagagaattgctagcttgctaataattctctagtggcgcacgggtgagtaacacgtgagtaacctgcccccgagagcgggatagccctgggaaactgggattaataccgcatagtatcgcaagattaaagcagcaatgcgcttggggatgggctcgcggcctattagttagttggtgaggtaacggctcaccaaggcgatgacgggtagccggtctgagaggatgtccggccacactggaactgagacacggtccagacacctacgggtggcagcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagggattagatacccctgtagtcctggcagtaaacggtgcacgcttggtgtgcggggaatcgaccccctgcgtgccggagctaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtaatgaacaacatgtgaaagcatgcgactcttcggaggcgttacacaggtgctgcatggccgtcgtcagctcgtgtcgtgagatgtttggttaagtccagcaacgagcgcaacccctgttgccagttaccagcacgtgaaggtggggactctggcgagactgcccagatcaactgggaggaaggtggggacgacgtcaggtcagtatggcccttatgcccagggctgcacacgtactacaatgcccagtacagagggggccgaagccgcgaggcggaggaaatcctaaaaactgggcccagttcggactgtaggctgcaacccgcctacacgaagccggaatcgctagtaatggcgcatcagctacggcgccgtgaatacgttcccgggtcttgtacacaccgcccgtcacatcatggaagccggtcgcacccgaagtatctgaagccaaccgcaaggaggcagggtcctaaggtgagactggtaactgggatgaagtcgtaacaaggtagccgtaggggaacctgcggctggatcacctcctttct

SEQ ID NO: 3 - DNA, 1504 bp, akkermansia muciniphila
ctggcggcgtggataagacatgcaagtcgaacgagagaattgctagcttgctaataattctctagtggcgcacgggtgagtaacacgtgagtaacctgcccccgagagcgggatagccctgggaaactgggattaataccgcatagtatcgaaagattaaagcagcaatgcgcttggggatgggctcgcggcctattagttagttggtgaggtaacggctcaccaaggcgatgacgggtagccggtctgagaggatgtccggccacactggaactgagacacggtccagacacctacgggtggcagcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagggattagatacccctgtagtcctggcagtaaacggtgcacgcttggtgtgcggggaatcgaccccctgcgtgccggagctaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtaatgaacaacatgtgaaagcatgcgactcttcggaggcgttacacaggtgctgcatggccgtcgtcagctcgtgtcgtgagatgtttggttaagtccagcaacgagcgcaacccctgttgccagttaccagcacgtgaaggtggggactctggcgagactgcccagatcaactgggaggaaggtggggacgacgtcaggtcagtatggcccttatgcccagggctgcacacgtactacaatgcccagtacagagggggccgaagccgcgaggcggaggaaatcctaaaaactgggcccagttcggactgtaggctgcaacccgcctacacgaagccggaatcgctagtaatggcgcatcagctacggcgccgtgaatacgttcccgggtcttgtacacaccgcccgtcacatcatggaagccggtcgcacccgaagtatctgaagccaaccgcaaggaggcagggtcctaaggtgagactggtaactgggatgaagtcgtaacaaggtagccgtaggggaacctgcggctggatcacctcctttctatggagcaagtgcacggaagtgcac

SEQ ID NO: 4 - DNA, 1438 bp, akkermansia muciniphila
tgctagcttgctaataattctctagtggcgcacgggtgagtaacacgtgagtaacctgcccccgagagcgggatagccctgggaaactgggattaataccgcatagtatcgcaagattaaagcagcaatgcgcttggggatgggctcgcggcctattagttagttggtgaggtaacggctcaccaaggcgatgacgggtagccggtctgagaggatgtccggccacactggaactgagacacggtccagacacctacgggtggcagcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagggattagatacccctgtagtcctggcagtaaacggtgcacgcttggtgtgcggggaatcgaccccctgcgtgccggagctaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtaatgaacaacatgtgaaagcatgcgactcttcggaggcgttacacaggtgctgcatggccgtcgtcagctcgtgtcgtgagatgtttggttaagtccagcaacgagcgcaacccctgttgccagttaccagcacgtgaaggtggggactctggcgagactgcccagatcaactgggaggaaggtggggacgacgtcaggtcagtatggcccttatgcccagggctgcacacgtactacaatgcccagtacagagggggccgaagccgcgaggcggaggaaatcctaaaaactgggcccagttcggactgtaggctgcaacccgcctacacgaagccggaatcgctagtaatggcgcatcagctacggcgccgtgaatacgttcccgggtcttgtacacaccgcccgtcacatcatggaagccggtcgcacccgaagtatctgaagccaaccgcaaggaggcagggtcctaaggtgagactggtaactgggatgaagtcgtaacaaggtagccgtaggggaacctgcggctggatcacctcctttc

SEQ ID NO: 5 - DNA, 1554 bp, akkermansia muciniphila
  misc_feature (1537)..(1542): n is a, c, g, or t
tccagcaatttcaaaaattaatttgatggagagtttgattctggctcagaacgaacgctggcggcgtggataagacatgcaagtcgaacgagagaattgctagcttgctaataattctctagtggcgcacgggtgagtaacacgtgagtaacctgcccccgagagcgggatagccctgggaaactgggattaataccgcatagtatcgcaagattaaagcagcaatgcgcttggggatgggctcgcggcctattagttagttggtgaggtaacggctcaccaaggcgatgacgggtagccggtctgagaggatgtccggccacactggaactgagacacggtccagacacctacgggtggcagcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagggattagatacccctgtagtcctggcagtaaacggtgcacgcttggtgtgcggggaatcgaccccctgcgtgccggagctaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtaatgaacaacatgtgaaagcatgcgactcttcggaggcgttacacaggtgctgcatggccgtcgtcagctcgtgtcgtgagatgtttggttaagtccagcaacgagcgcaacccctgttgccagttaccagcacgtgaaggtggggactctggcgagactgcccagatcaactgggaggaaggtggggacgacgtcaggtcagtatggcccttatgcccagggctgcacacgtactacaatgcccagtacagagggggccgaagccgcgaggcggaggaaatcctaaaaactgggcccagttcggactgtaggctgcaacccgcctacacgaagccggaatcgctagtaatggcgcatcagctacggcgccgtgaatacgttcccgggtcttgtacacaccgcccgtcacatcatggaagccggtcgcacccgaagtatctgaagccaaccgcaaggaggcagggtcctaaggtgagactggtaactgggatgaagtcgtaacaaggtagccgtaggggaacctgcggctggatcacctcctttctnnnnnnatggagcaagta

SEQ ID NO: 6 - DNA, 1507 bp, akkermansia muciniphila
gagtttgattctggctcagaacgaacgctggcggcgtggataagacatgcaagtcgaacgagagaattgctagcttgctaataattctctagtggcgcacgggtgagtaacacgtgagtaacctgcccccgagagcgggatagccctgggaaactgggattaataccgcatagtatcgaaagattaaagcagcaatgcgcttggggatgggctcgcggcctattagttagttggtgaggtaacggctcaccaaggcgatgacgggtagccggtctgagaggatgtccggccacactggaactgagacacggtccagacacctacgggtggcagcagtcgagaatcattcacaatgggggaaaccctgatggtgtgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagggattagatacccctgtagtcctggcagtaaacggtgcacgcttggtgtgcggggaatcgaccccctgcgtgccggagctaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtaatgaacaacatgtgaaagcatgcgactcttcggaggcgttacacaggtgctgcatggccgtcgtcagctcgtgtcgtgagatgtttggttaagtccagcaacgagcgcaacccctgttgccagttaccagcacgtgaaggtggggactctggcgagactgcccagatcaactgggaggaaggtggggacgacgtcaggtcagtatggcccttatgcccagggctgcacacgtactacaatgcccagtacagagggggccgaagccgcgaggcggaggaaatcctgaaaactgggcccagttcggactgtaggctgcaacccgcctacacgaagccggaatcgctagtaatggcgcatcagctacggcgccgtgaatacgttcccgggtcttgtacacaccgcccgtcacatcatggaagccggtcgcacccgaagtatctgaagccaaccgcaaggaggcagggtcctaaggtgagactggtaactgggatgaagtcgtaacaaggtagccgtaggggaacctgcggctggatcacctcctttcta